SEQUENCE LISTING (45 sequences)

SEQ ID NO: 1 - DNA, 10 bp, Artificial sequence
  Activated p53 recognision sequence in p53-target gene promoter
  misc_feature (1)..(1): n is either A or G
  misc_feature (2)..(2): n is either A or G
  misc_feature (3)..(3): n is either A or G
  misc_feature (5)..(5): n is either A or T
  misc_feature (6)..(6): n is either A or T
  misc_feature (8)..(8): n is either C or T
  misc_feature (9)..(9): n is either C or T
  misc_feature (10)..(10): n is either C or T
nnncnngnnn

SEQ ID NO: 2 - DNA, 20 bp, Artificial sequence
  p53 target gene promoter DNA
gaacatgtcccaacatgttg

SEQ ID NO: 3 - DNA, 10 bp, Artificial sequence
  p53-target gene promoter-2 DNA
gggcatgtct

SEQ ID NO: 4 - DNA, 141 bp, Artificial sequence
  Left ITR DNA sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 141 bp, Artificial sequence
  Right-ITR DNA sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 2369 bp, Artificial sequence
  5′ flanking region of p21 gene
aagcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagctt

SEQ ID NO: 7 - DNA, 7181 bp, Artificial sequence
  Plasmid PLS
ggtaccgagctcttacgcgtgctagcccgggctcgagatctgcgatctaagtaagcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 8 - DNA, 2369 bp, Artificial sequence; identical to SEQ ID NO: 6
  5′ flanking region of p21 gene in plasmid PLS

SEQ ID NO: 9 - DNA, 1653 bp, Artificial sequence
  Luciferase-coding sequence in plasmid PLS
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 10 - DNA, 222 bp, Artificial sequence
  SV40-derived polyadenylation signal in plasmid PLS
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 11 - DNA, 7230 bp, Artificial sequence
  Plasmid pAAV-PLS
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctcgagatctgcgatctaagtaagcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 141 bp, Artificial sequence; identical to SEQ ID NO: 4
  Left-ITR in plasmid pAAAV-PLS

SEQ ID NO: 13 - DNA, 2369 bp, Artificial sequence; identical to SEQ ID NO: 6
  5′ flanking region of p21 gene in plasmid pAAV-PLS

SEQ ID NO: 14 - DNA, 1653 bp, Artificial sequence; identical to SEQ ID NO: 9
  Luciferase-coding sequence in plasmid pAAV-PLS

SEQ ID NO: 15 - DNA, 222 bp, Artificial sequence; identical to SEQ ID NO: 10
  SV40-derived polyadenylation signal in plasmid pAAV-PLS

SEQ ID NO: 16 - DNA, 141 bp, Artificial sequence; identical to SEQ ID NO: 5
  Right-ITR in plasmid pAAV-PLS

SEQ ID NO: 17 - DNA, 7327 bp, Artificial sequence
  Plasmid pAAV-RC
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 18 - DNA, 11635 bp, Artificial sequence
  Plasmid pHelper
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 19 - DNA, 4633 bp, Artificial sequence
  Plasmid rAAV-PLS
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctcgagatctgcgatctaagtaagcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 141 bp, Artificial sequence; identical to SEQ ID NO: 4
  Left-ITR in plasmid rAAV-PLS

SEQ ID NO: 21 - DNA, 2369 bp, Artificial sequence; identical to SEQ ID NO: 6
  5′ flanking region of p21 gene in plasmid rAAV-PLS

SEQ ID NO: 22 - DNA, 1653 bp, Artificial sequence; identical to SEQ ID NO: 9
  Luciferase-coding sequence in plasmid rAAV-PLS

SEQ ID NO: 23 - DNA, 222 bp, Artificial sequence; identical to SEQ ID NO: 10
  SV40-derived polyadenylation signal in plasmid rAAV-PLS

SEQ ID NO: 24 - DNA, 141 bp, Artificial sequence; identical to SEQ ID NO: 5
  Right-ITR in plasmid rAAV-PLS

SEQ ID NO: 25 - DNA, 25 bp, Artificial sequence
  rAAV-PLS-specific primer
tcctggagagtgccaactcattctc

SEQ ID NO: 26 - DNA, 26 bp, Artificial sequence
  rAAV-PLS-specific primer2
ttccaggaaccagggcgtatctcttc

SEQ ID NO: 27 - DNA, 2147 bp, Artificial sequence
  Probe for rAAV-PLS
agtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtact

SEQ ID NO: 28 - DNA, 1145 bp, Artificial sequence
  Fragment containing HSV-tk-coding sequence in plasmid pORF-HSVtk
ccatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagaattcgctagc

SEQ ID NO: 29 - DNA, 7230 bp, Artificial sequence; identical to SEQ ID NO: 11
  Plasmid pAAV-PLS2

SEQ ID NO: 30 - DNA, 6713 bp, Artificial sequence
  Plasmid pAAV-PtkS
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctcgagatctgcgatctaagtaagcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagcttggcattccggtactgttggtaaagccaccatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagaattcgctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 31 - DNA, 141 bp, Artificial sequence; identical to SEQ ID NO: 4
  Left-ITR in plasmid pAAV-PtkS

SEQ ID NO: 32 - DNA, 2369 bp, Artificial sequence; identical to SEQ ID NO: 6
  5′ flanking region of p21 gene in plasmid pAAV-PtkS

SEQ ID NO: 33 - DNA, 1131 bp, Artificial sequence
  HSV-tk-coding sequence in plasmid pAAV-PtkS
atggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactga

SEQ ID NO: 34 - DNA, 222 bp, Artificial sequence; identical to SEQ ID NO: 10
  SV40-derived polyadenylation signal in plasmid pAAV-PtkS

SEQ ID NO: 35 - DNA, 141 bp, Artificial sequence; identical to SEQ ID NO: 5
  Right-ITR in plasmid pAAV-PtkS

SEQ ID NO: 36 - DNA, 4116 bp, Artificial sequence
  Plasmid rAAV-PtkS
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctcgagatctgcgatctaagtaagcttcccaggaacatgcttgggcagcaggctgtggctctgattggctttctggccgtcaggaacatgtcccaacatgttgagctctggcatagaagaggctggtggctattttgtccttgggctgcctgttttcaggtgaggaaggggatggtaggagacaggagacctctaaagaccccaggtaaaccttagcctgttactctgaacagggtatgtgatctgccagcagatccttgcgacagggctgggatctgatgcatgtgtgcttgtgtgagtgtgtgctgggagtcagattctgtgtgtgacttttaacagcctgctcccttgcctttttcagggcagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggaagcttggcattccggtactgttggtaaagccaccatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagaattcgctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 37 - DNA, 141 bp, Artificial sequence; identical to SEQ ID NO: 4
  Left-ITR in plasmid rAAV-PtkS

SEQ ID NO: 38 - DNA, 2369 bp, Artificial sequence; identical to SEQ ID NO: 6
  5′ flanking region of p21 gene in plasmid rAAV-PtkS

SEQ ID NO: 39 - DNA, 1131 bp, Artificial sequence; identical to SEQ ID NO: 33
  HSV-tk-coding sequence in plasmid rAAV-PtkS

SEQ ID NO: 40 - DNA, 222 bp, Artificial sequence; identical to SEQ ID NO: 10
  SV40-derived polyadenylation signal in plasmid rAAV-PtkS

SEQ ID NO: 41 - DNA, 141 bp, Artificial sequence; identical to SEQ ID NO: 5
  Right-ITR in plasmid rAAV-PtkS

SEQ ID NO: 42 - DNA, 20 bp, Artificial sequence
  HSV-tk-specific primer
cggagcagaaaatgcccacg

SEQ ID NO: 43 - DNA, 20 bp, Artificial sequence
  HSV-tk-specific primer 2
tgctgcccataaggtaccgc

SEQ ID NO: 44 - DNA, 20 bp, Artificial sequence
  Actin-specific primer
gtagccatccaggctgtgtt

SEQ ID NO: 45 - DNA, 20 bp, Artificial sequence
  Actin-specific primer2
cagtgaggccaggatagagc

(19) United States
(12) Patent Application Publication
(10) Pub. No.: US 2008/0019946 A1
(43) Pub. Date: Jan. 24, 2008

(54) Integration-type low-dose radiation-inducible vector

(75) Inventors: Mitsuru Nenoi, Chiba (JP); Kazuhiro Daino, Chiba (JP)

Correspondence Address: WELSH & KATZ, LTD, 120 S RIVERSIDE PLAZA, 22ND FLOOR, CHICAGO, IL, 60606, US

(73) Assignee: National Institute of Radiological Sciences, Chiba (JP)

(21) Appl. No.: 11/788,256

(22) Filed: Apr. 19, 2007

Related U.S. Application Data
(63) Continuation of application No. PCT/JP05/11088, filed on Jun. 10, 2005.

(30) Foreign Application Priority Data
Oct. 20, 2004 (JP) 2004-305870

(51) Int. Cl. A61K 48/00; A61P 35/00; C12N 15/00

(52) U.S. Cl. 424/93.2; 435/320.1

(57) ABSTRACT

An integration-type low-dose radiation-inducible viral vector comprising a DNA sequence comprising a p53 target gene promoter sequence and a therapeutic gene sequence. The vector of the present invention is useful for gene therapy.

### TECHNICAL FIELD

The present invention relates to an integration-type low-dose radiation-inducible vector useful in gene therapy, a pharmaceutical composition for use in gene therapy comprising the vector, and a gene therapy method using the pharmaceutical composition.

### BACKGROUND ART

In gene therapy, the technique for delivering a therapeutic gene to a target site is important. For instance, in gene therapy of cancer, the technique for delivering accurately to the foci a therapeutic gene functioning in the growth inhibition of cancer is important.

Tissue-specific receptors, promoter sequences/enhancers and the like, are used in order to accomplish such gene deliveries. For instance, using the promoter sequence of the carcinoembryonic antigen gene and placing the therapeutic gene under control of the promoter sequence, the therapeutic gene can be expressed selectively in large intestine cancer and lung cancer cells producing carcinoembryonic antigen.

Among these delivery means, the delivery of a therapeutic gene to cancer tissue using the promoter sequence of a radiation-inducible gene is an effective means. The reason is, for a therapeutic gene placed under the control of the promoter sequence of a radiation-inducible gene, as the expression thereof is induced by irradiation (radiation inducible), selective gene expression at the site where the radiation was irradiated becomes possible. Therefore, the expression of the therapeutic gene can be controlled with good accuracy, spatially and temporally, by combining the promoter sequence of a radiation-inducible gene and the stereotactic irradiation technique, which is under remarkable progress in the field of radiation therapy.

The promoter sequence of early growth response gene Egr-1 is a promoter sequence for which research on a promoter sequence of a radiation-inducible gene for use in gene therapy is most advanced. “TNFerade”, a vector in which the cytokine TNFα gene is linked as the therapeutic gene under the control of the Egr-1 gene promoter sequence has been developed, for which Phase I clinical study has been completed, and currently a Phase II clinical study is under way (for instance, refer to Senzer N, et al., J Clin Oncol, 22, 592-601, 2004, TNFerade biologic, an adenovector with a radiation-inducible promoter, carrying the human tumor necrosis factor alpha gene: a phase I study in patients with solid tumors). TNFerade is a non-chromosomal integration-type vector based on adenovirus.

However, high doses of irradiation are necessary in order to express significantly the therapeutic gene using the Egr-1 gene promoter sequence. Actually, there is no report of success case by irradiation of 2 Gy or less. Such a high dose irradiation is not desirable for a gene therapy combining radiation therapy. The reason is, since a gene therapy that uses a therapeutic gene placed under the control of a radiation-inducible gene promoter sequence requires a given period of time until the therapeutic gene is expressed due to the irradiation and the therapeutic gene product accumulates at the foci and exerts a therapeutic effect, when the expression of the therapeutic gene is induced by a high dose irradiation, a time difference occurs between the time when therapeutic effect due to high dose radiation (effect due to radiation therapy) appears and the time when the therapeutic effect due to therapeutic gene product (effect due to gene therapy) appears, no synergistic effect caused by the combination of radiation therapy and gene therapy can be obtained. In addition, irradiating a high dose of radiation for the expression of therapeutic gene and then further carrying out radiation therapy by irradiation is not desirable, as the burden of the patient becomes bic.

Meanwhile, a system involving p53, which is the product of a tumor suppressor gene, is known as a low-dose radiation-inducible gene expression system. It is known that, in such a system, p53 activated by a low-dose irradiation acts on the promoter sequence of the target gene thereof (p53 target gene promoter sequence) to activate the expression of the p53 target gene, which is under the control of the promoter sequence (for instance, refer to Amundson S A, et al., Mol Cancer Res, 1, 445-452, 2003, Differential responses of stress genes to low-dose-rate gamma irradiation). Furthermore, vectors for use in gene therapy comprising the p53 target gene promoter sequence have also been developed (for instance, refer to Worthington J, Robson T, Murray M, O'Rourke M, Keilty G, Hirst D G. Modification of vascular tone using iNOS under the control of a radiation-inducible promoter. Gene Ther 2000; 7: 1126-1131; and Worthington J, Robson T, O'Keeffe M, Hirst D G. Tumour cell radiosensitization using constitutive (CMV) and radiation-inducible (WAF1) promoters to drive the iNOS gene: a novel suicide gene therapy. Gene Ther 2002; 9: 263-269). The above vectors for use in gene therapy comprising the p53 target gene promoter sequence are non-chromosomal integration-type vectors based on non-viral vectors (cationic liposomes and polysomes).

However, the above non-integration-type vectors comprising the p53 target gene promoter sequence cannot be satisfactory as vectors for use in gene therapy, owing to a low inducibility of the expression of the therapeutic gene by irradiation.

### DISCLOSURE OF THE INVENTION

Based on reports suggesting that induction of the expression of the p53 target gene by p53 is related to a mechanism depending on a high order chromosome structure (for instance, refer to Espinosa J M, Emerson B M. Transcriptional regulation by p53 through intrinsic DNA/chromatin binding and site-directed cofactor recruitment. Mol Cell 2001; 8: 57-69; and Braastad C D, Han Z, Hendrickson E A. Constitutive DNase I hypersensitivity of p53-regulated promoters. J Biol Chem 2003; 278: 8261-8268), the present inventors carried out earnest studies focusing on the p53 target gene promoter sequence introduced into the host cell and the state of presence of the p53 target gene (therapeutic gene) in the host cell. As a result, they found that when the p53 target gene promoter sequence and a therapeutic gene are introduced into a host cell using the adeno-associated virus (AAV) vector, which is a chromosome integration-type vector, the expression of the therapeutic gene is induced at a high level with a low-dose irradiation. The present invention was made based on this observation.

That is to say, the present invention relates to

(1) a vector, which is an integration-type low-dose radiation-inducible viral vector,

comprising a DNA sequence comprising a p53 target gene promoter sequence and a therapeutic gene sequence;

(2) a pharmaceutical composition, which is a pharmaceutical composition for use in gene therapy to treat a disease treatable by gene therapy, comprising the above viral vector; as well as

(3) a gene therapy method, comprising the steps of:

(i) providing a pharmaceutical composition comprising the viral vector;

(ii) administering the pharmaceutical composition to a patient having a disease treatable by gene therapy; and

(iii) irradiating a site requiring the expression of the therapeutic gene on the patient, with a dose of radiation sufficient to express the DNA sequence of the vector integrated in the chromosome of the patient.

### BRIEF DESCRIPTION OF THE DRAWINGS

FIG. 1 is a schematic showing the genomic structure of the integration-type low-dose radiation-inducible viral vector rAAV-PLS of the present invention;

FIG. 2 shows the dose dependency of the rate of induction of the luciferase gene expression obtained in Example 3 and Comparative Example 1;

FIG. 3 shows the results of PCR analysis of transduced MCF-7 cell genomic DNA using rAAV-PLS-specific primers;

FIG. 4 shows the results of Southern blot analysis of transduced MCF-7 cell genomic DNA using restriction enzymes;

FIG. 5 is a schematic showing the genomic structure of the integration-type low-dose radiation-inducible viral vector rAAV-PtkS of the present invention;

FIG. 6 shows the results of RT-PCR for the expression of the HSV-tk gene and an actin gene in transduced MCF-7 cell; and

FIG. 7 shows the relative number of cells surviving after X-ray irradiation for HSV-tk transgenic MCF-7 cells (PtkS-1 and PtkS-2) as well as luciferase transgenic MCF-7 cells (PLS).

### BEST MODE FOR CARRYING OUT THE INVENTION

Hereinafter, the present invention will be described in detail.

The integration-type low-dose radiation-inducible viral vector of the present invention contains a DNA sequence comprising a p53 target gene promoter sequence and a therapeutic gene sequence.

The viral vector of the present invention is a chromosome integration-type viral vector enabling integration of the DNA sequence thereof into the chromosome of the host cell. In the present specification, the aforementioned chromosome integration-type viral vector is simply called integration-type viral vector.

The integration-type viral vector can be constructed based on an integration-type virus.

Retroviruses and parvoviruses are integration-type viruses that can be used in the present invention. Lentivirus can be exemplified as the retrovirus, and the adeno-associated virus can be exemplified as the parvovirus. Among these the lentivirus and the adeno-associated virus are preferred. The adeno-associated virus is particularly preferred, as it is nonpathogenic and highly safe, and furthermore, it has a broad spectrum of hosts, enabling gene introduction not only to dividing cells, but also to non-dividing cells.

The adeno-associated virus is a virus that belongs to the parvovirus family, which contains a linear single strand DNA within a capsid. Types 1 to 8 can be exemplified as the adeno-associated virus, with type 2 and type 8 being preferred, and type 2 being particularly preferred.

The “p53 target gene promoter sequence” contained in the DNA sequence of the viral vector of the present invention means a promoter sequence upon which p53 activated by a low-dose irradiation acts and allows the expression to be activated, of the therapeutic gene under the control of the promoter sequence.

The “p53 target gene promoter sequence” has the following sequence as the activated p53 recognition sequence:
NNNCNNGNNN (SEQ ID NO: 1)
(where N represents A, G, T, and C)

Preferably, the “p53 target gene promoter sequence” has the following sequence:

| | |
|---|---|
| GAACATGTCCCAACATGTTG | (SEQ ID NO:2) |
| and/or | |
| GGGCATGTCT | (SEQ ID NO:3) |

The p21 gene promoter sequence, the MDM2 gene promoter sequence, the GADD45 gene promoter sequence, the 14-3-3σ gene promoter sequence, the KARP-1 gene promoter sequence, the BAX gene promoter sequence, the DR5 gene promoter sequence, the BID gene promoter sequence, the PUMA gene promoter sequence, the NOXA gene promoter sequence, and the like, can be exemplified as the p53 target gene promoter sequence that can be used in the present invention. Among these, the GADD45 gene promoter sequence and the p21 gene promoter sequence are preferred, and the p21 gene promoter sequence is particularly preferred. These p53 target gene promoter sequences are publicly-known. For instance, the promoter sequence of the p21 gene is disclosed in the DNA base sequence database (GenBank) as accession number Z85996.

The “therapeutic gene sequence” contained in the DNA sequence of the viral vector of the present invention means a sequence coding for a gene product that exerts a therapeutic effect inside a host cell during gene therapy. There is no limitation on the therapeutic gene as long as it is a gene that is effective in the treatment of the disease that is the target of the gene therapy. For instance, when the target of the gene therapy is cancer, the TNFα gene, apoptosis inducing protein genes, tumor suppressor protein genes, angiogenic inhibitor protein genes, antisense nucleic acid genes, prodrug activator genes, radiosensitizer genes, and the like, can be exemplified as therapeutic genes. Among these, the TNFα gene and prodrug activator genes are preferred, the prodrug activator genes being particularly preferred. As an example of prodrug activator gene, the HSV-tk gene can be exemplified, which codes for the herpes simplex virus thymidine kinase (HSV-tk) that allows the prodrug ganciclovir to be activated and exert an inhibitory action of DNA synthesis. The sequences of these therapeutic genes are publicly-known. For instance, the sequence of a prodrug activator gene is disclosed in the DNA base sequence database (GenBank) as accession number V00470.

Note that the size of the DNA sequence to be inserted into the viral vector (that is to say, the total size of the p53 target gene promoter sequence and the therapeutic gene sequence (when a Left-ITR, a polyadenylation signal sequence, a Right-ITR, and the like, described below, are included, the size also including these)) must be within the size limits allowed by the viral vector receiving the insertion. For instance, in the case of the adeno-associated virus vector, the size of the DNA sequence to be inserted must be 4.7 kb or less.

In addition, the therapeutic gene sequence must be linked, in a state allows expression under the control of the p53 target gene promoter sequence, to the promoter sequence.

The number strands of the DNA sequence of the viral vector of the present invention can be changed depending on the species of the virus on which the vector is based. For instance, when based on the adeno-associated virus, the DNA sequence of the viral vector is single stranded sequence.

The viral vector of the present invention exists in the state of a viral particle comprising the above-mentioned DNA sequence within a capsid. For instance, when based on the adeno-associated virus, the viral vector of the present invention has an icosahedral capsid of approximately 20 nm diameter.

The viral vector of the present invention has “low-dose radiation-inducibility”. Having “low-dose radiation-inducibility” means allowing the therapeutic gene expression activity to be raised, after integrating the DNA sequence of the viral vector into the chromosome of the host cell and irradiating with a 1 Gy radiation, by at least 100% compared to the expression activity when not irradiated, and preferably at least 200%.

The viral vector of the present invention can be constructed according to general viral vector construction methods, for instance, methods described in references: Xiao X, Li J, Samulski R J. Production of high-titer recombinant adeno-associated virus vectors in the absence of helper adenovirus. J Virol 1998; 72: 2224-2232, and Matsushita T et al. Adeno-associated virus vectors can be efficiently produced without helper virus. Gene Ther 1998; 5: 938-945.

In one embodiment, the integration-type low-dose radiation-inducible viral vector of the present invention contains a DNA sequence comprising

(a) a Left-ITR,

(b) a p53 target gene promoter sequence,

(c) a therapeutic gene sequence,

(d) a polyadenylation signal sequence and

(e) a Right-ITR,

from the five prime end side to the three prime end side, in the order of (a), (d), (c), (b), (e).

The (b) p53 target gene promoter sequence and (c) therapeutic gene sequence contained in the DNA sequence of the viral vector of the above one embodiment are the same as those described previously.

The (a) “Left-ITR” and (b) “Right-ITR” contained in the DNA sequence of the viral vector of the above one embodiment respectively mean sequences also called inverted terminal repeats. The “Left-ITR” and “Right-ITR” respectively contain complementary base sequences in opposite directions, and can adopt a T-shaped hairpin structure. The Left-ITR and Right-ITR are considered to play an important role in the integration of the viral DNA into host cell's chromosome. For instance, when an adeno-associated virus is used as the basis of a viral vector, sequences indicated by SEQ ID Nos. 4 and 5 can be used as Left-ITR and Right-ITR, respectively.

Left-ITR:

(SEQ ID NO:4)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT
Right-ITR:

(SEQ ID NO:5)

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG

The (d) “polyadenylation signal sequence” contained in the DNA sequence of the viral vector of the above one embodiment means a sequence coding for a region recognized by the poly (A) polymerase, which has the function of adding a polyadenylic acid to an mRNA. The polyadenylation signal sequence is considered to be helpful in stabilization of the mRNA of the therapeutic gene transcribed inside the host cell. The “polyadenylation signal sequence” has the following sequence: AATAAA. As specific examples, the SV40-derived polyadenylation signal sequence, the human somatotrophic hormone gene-derived polyadenylation signal sequence, the human beta globin gene-derived polyadenylation signal sequence, and the like, can be exemplified. Among these, the SV40-derived polyadenylation signal sequence and the human beta globin gene-derived polyadenylation signal sequence are preferred, the SV40-derived polyadenylation signal sequence being particularly preferred. These polyadenylation signal sequences are publicly-known. For instance, the SV40-derived polyadenylation signal sequence is described in reference: Levitt N, Briggs D, Gil A, Proudfoot N J. Definition of an efficient synthetic poly (A) site. GENES DEV. 1989 7: 1019-25.

The DNA sequence of the viral vector of the above one embodiment contains the sequences: (a) Left-ITR, (b) p53 target gene promoter sequence, (c) therapeutic gene sequence, (d) polyadenylation signal sequence and (e) Right-ITR, from the five prime end side to the three prime end side, in the order of (a), (d), (c), (b), (e). Note that, therapeutic gene sequence must be linked, in a state that allows expression under the control of the p53 target gene promoter sequence, to the promoter sequence.

In another embodiment, (b) a complementary sequence to the p53 target gene promoter sequence, (c) a complementary sequence to the therapeutic gene sequence and (d) a complementary sequence to the polyadenylation signal sequence can also be used as sequences contained between the Left-ITR and the Right-ITR. In this case, the DNA sequence of the viral vector contains the sequences: (a) Left-ITR, (d) complementary sequence to the polyadenylation signal sequence, (c) complementary sequence to the therapeutic gene sequence, (b) complementary sequence to the p53 target gene promoter sequence and (e) Right-ITR, from the five prime end side to the three prime end side in the order of (a), (d), (c), (b), (e).

In addition to the above sequences, the DNA sequence of the viral vector of the present invention may contain a polyadenylation signal sequence in the promoter upstream region. The polyadenylation signal sequence in the promoter upstream region is useful from the point of background suppression during non-irradiation. As specific examples, the synthetic polyadenylation signal sequence and the human somatotrophic hormone gene-derived polyadenylation signal sequence can be exemplified. Among these, the synthetic polyadenylation signal sequence is particularly preferred.

For instance, when the viral vector of the above one embodiment is based on the adeno-associated virus, the viral vector can be constructed by a triple transfection method comprising the following steps.

(1) a step of creating three species of plasmids: (α) a vector plasmid with the p53 target gene promoter sequence, a therapeutic gene and the polyadenylation signal sequence inserted between the Left-ITR and the Right-ITR of the wild type adeno-associated virus, (β) a helper plasmid comprising genes (rep and cap) required for virus reproduction and viral particle formation, as well as (γ) an adenovirus gene expression plasmid comprising adenovirus genes (E2A, E4 and VA) required for adeno-associated virus vector production;

(2) a step of co-transfecting 293 cells (comprising the E1A and E1B genes) with the three species of plasmids,

(3) a step of incubating the cells to produce and accumulate the viral vectors in the 293 cells,

(4) a step of freeze-thawing the cells to recover the viral vectors, and

(5) as necessary, a step of purifying and/or concentrating the recovered viral vectors by a density gradient ultracentrifugation method using cesium chloride or an affinity chromatography method.

The pharmaceutical composition of the present invention comprises the above integration-type low-dose radiation-inducible viral vector and can be used in the treatment of a disease treatable by gene therapy.

The subject of application of the pharmaceutical composition of the present invention is a disease treatable by gene therapy. As specific examples, cancers, restenosis, ischaemic cardiac diseases, arteriosclerosis, and the like, can be exemplified. Among these diseases, the pharmaceutical composition of the present invention can exert particularly excellent therapeutic effects on cancers, on the point of obtaining synergistic effects by the combined application of gene therapy and radiation therapy. As applicable cancers, breast cancers, prostate gland cancers and brain tumors can be exemplified. Among these cancers, the pharmaceutical composition of the present invention can exert excellent therapeutic effects on breast cancers.

The pharmaceutical composition of the present invention may contain the integration-type low-dose radiation-inducible viral vector alone as the active ingredient, or may further contain other active substances. Prodrugs, radiosensitizers, immuno-stimulators, and the like, can be exemplified as other active substances.

The pharmaceutical composition of the present invention may contain a pharmaceutically acceptable carrier. Water, physiological saline, buffer solutions, and the like, can be exemplified as the pharmaceutically acceptable carriers.

As dosage forms of the pharmaceutical composition of the present invention, injectables directly applied into the body (including suspensions and emulsions), and the like, can be exemplified.

The pharmaceutical composition of the present invention can be prepared by formulation methods that are well known in the art.

The gene therapy method of the present invention generally comprises the following steps:

(1) a step of providing a pharmaceutical composition comprising an integration-type low-dose radiation-inducible viral vector comprising a DNA sequence comprising a p53 target gene promoter sequence and a therapeutic gene sequence,

(2) a step of administering the pharmaceutical composition to a patient having a disease treatable by gene therapy, and

(3) a step of irradiating a site in the patient where expression of the therapeutic gene is necessary, with a dose of radiation sufficient to express the DNA sequence of the vector integrated in the chromosome of the patient.

The above step in (1) can be carried out according to the description regarding the above low-dose radiation-inducible viral vector and the pharmaceutical composition comprising the vector.

In the above step in (2), the diseases that are the subjects of the treatment are the same as the above diseases that are the subjects of the application of the pharmaceutical composition of the present invention.

For administration of the pharmaceutical composition to patient, direct administration method whereby the therapeutic gene is introduced in vivo and autotransplantation method whereby the therapeutic gene is introduced ex vivo can be exemplified.

With the direct administration method, the pharmaceutical composition comprising the vector is injected directly into the patient. In so doing, although systemic administration by venous injection, arterial injection, or the like, is also possible, in situ local administration to the foci is preferred, as immunoreaction against the vector can be minimized.

With the autotransplantation method, cells collected from foci of the patient are treated extracorporally with the pharmaceutical composition comprising the vector, thereafter, cells with the DNA sequence of the vector integrated in the chromosome are returned back into the patient.

The dosage of the pharmaceutical composition varies in general depending on the type of the disease and the state of the patient. For instance, in the case of a breast cancer, it is 10⁸ to 10¹¹ in terms of viral vectors, preferably 10⁹ to 10¹¹, particularly preferably 10¹⁰ to 10¹¹ per administration per adult patient.

In addition, the number of administration may be once to twice daily, and the administration period may span one day to 5 days or longer; taking 1 to 10 administrations as one set, multiple sets may be administered intermittently over a long period of time.

Note that the pharmaceutical composition of the present invention is administered after examining the state of the p53 gene at the foci of the patient and verifying that the p53 gene is functioning properly at the foci.

X-ray, gamma ray, particle ray, and the like, can be exemplified as the type of radiation beam irradiated in the step of (3) above, gamma ray and particle ray being preferred, and particle ray being particularly preferred.

It suffices that the exposure dose is a dose that is sufficient to express the DNA sequence of the vector integrated in the chromosome of the patient, in general, 0.5 to 2 Gy, and preferably 0.5 to 1 Gy.

As for the number of irradiation, it suffices that irradiation with the above dose is carried out once, and it may be carried out 2 to 3 times or more, as necessary.

Using stereotactic irradiation techniques, it is possible to carry out the irradiation only in regions requiring expression of the DNA sequence of integrated vector. Stereotactic irradiation can be carried out, for instance, using the apparatus: HIMAC (manufacturer name: National Institute of Radiological Sciences).

Regarding the time interval after administration of the pharmaceutical composition according to step (2) until irradiation of radiation according to step (3), sufficient time that the DNA sequence of the vector is integrated into the chromosome of the patient, is required. Such time interval varies depending on the type of disease and the state of the patient, or the like. For instance, in the case of breast cancer, it is 5 to 9 weeks, preferably 5 to 7 weeks, and particularly preferably 5 to 6 weeks.

If the disease targeted for treatment is cancer, the gene therapy method of the present invention may comprise, subsequently to the above steps (1) to (3), the following step:

(4) a step of irradiating the site expressing the therapeutic gene with a dose of radiation sufficient to treat cancer.

That is to say, the cancer gene therapy method of the present invention comprises the following steps:

(1) a step of providing a pharmaceutical composition comprising an integration-type low-dose radiation-inducible viral vector comprising a DNA sequence comprising a p53 target gene promoter sequence and a therapeutic gene sequence,

(2) a step of administering the pharmaceutical composition to a cancer patient,

(3) a step of irradiating cancer foci of the patient with a dose of radiation sufficient to express the DNA sequence of the vector integrated in the chromosome of the patient, and

(4) a step of irradiating the site expressing the therapeutic gene with a dose of radiation sufficient to treat cancer.

Except that the target disease is cancer and that the site of irradiation is cancer foci, the steps (1) to (3) of the cancer gene therapy method of the present invention are identical to steps (1) to (3) of the above general gene therapy method.

X-ray, gamma ray, particle ray, and the like, can be exemplified as the type of radiation beam irradiated in the step of (4) above, gamma ray and particle ray being preferred, and particle ray being particularly preferred.

It suffices that the exposure dose is a dose that is sufficient to treat cancer. The exposure dose varies depending on the type of cancer and the state of the patient, or the like; for instance, in the case of breast cancer, it is in general 10 to 60 Gy, preferably 10 to 30 Gy, and particularly preferably 10 to 20 Gy.

As for the number of irradiation, it varies depending on the type of cancer and the state of the patient, or the like, and for instance, in the case of breast cancer, it is in general 30 times.

Using stereotactic irradiation techniques, it is possible to carry out the irradiation only at cancer foci. Stereotactic irradiation can be carried out, for instance, using the apparatus: HIMAC (manufacturer name: National Institute of Radiological Sciences).

Regarding the time interval after irradiation with a radiation dose sufficiently to express the DNA sequence of the vector integrated into the chromosome of the patient according to step (3) until irradiation according to step (4), sufficient time that the therapeutic gene is expressed inside the body of the patient due to the irradiation in step (3) and the therapeutic gene product accumulates at the cancer foci, is required. Such time interval varies depending on the type of cancer and the state of the patient, or the like, and for instance, in the case of breast cancer, it is 3 to 12 hours, preferably 3 to 8 hours, and particularly preferably 3 to 6 hours.

By carrying out the step (4), a synergistic therapeutic effect combining the therapeutic effects from the gene therapy of steps (1) to (3) and the therapeutic effects of radiation therapy due to step (4) is obtained.

Although the targets of the above gene cancer therapy method are mammals, in particular human, animal experiments carried out prior to establishing therapy with human as subjects comprise, for instance, the following steps:

(1) a step of transplanting breast cancer cells to a nude mouse to create a mouse model for cancer,

(2) a step of providing a pharmaceutical composition comprising an integration-type low-dose radiation-inducible viral vector comprising the DNA sequence comprising the p53 target gene promoter sequence and the herpes simplex virus thymidine kinase gene serving as the therapeutic gene,

(3) a step of injecting the pharmaceutical composition into cancer focal sites of the model mouse,

(4) a step of irradiating cancer focal site of the model mouse with a dose of radiation sufficient to express the DNA sequence of the vector integrated in the chromosome of the mouse,

(5) a step of administering ganciclovir (a prodrug that demonstrates cytotoxicity by the action of herpes simplex virus thymidine kinase) intraperitoneally to the model mouse, and

(6) a step of irradiating the site expressing the therapeutic gene with a dose of radiation sufficient to treat cancer.

In the following, a specific description will be provided giving examples; however, the present invention is not limited by the examples.

### EXAMPLES

### Example 1

### Integration-Type Low-Dose Radiation-Inducible Viral Vector

In the present example, a low-dose radiation-inducible viral vector rAAV-PLS was constructed based on a type 2 adeno-associated virus, having the p21 gene promoter sequence as the p53 target gene promoter sequence, and having the luciferase gene sequence corresponding to the therapeutic gene sequence. rAAV-PLS was constructed by the triple transfection method (Xiao X, Li J, Samulski R J. Production of high-titer recombinant adeno-associated virus vectors in the absence of helper adenovirus. J Virol 1998; 72: 2224-2232. and Matsushita T, Elliger S, Elliger C, Podsakoff G, Villarreal L, Kurtzman G J, Iwaki Y, Colosi P. Adeno-associated virus vectors can be efficiently produced without helper virus. Gene Ther 1998; 5: 938-945.) using the AAV Helper Free System (Stratagene).

First, HindIII was used to excise from the plasmid wwp-Luc (El-Deiry W S, Tokino T, Velculescu V E, Levy D B, Parsons R, Trent J M, Lin D, Mercer W E, Kinzler K W, Vogelstein B. WAF1, a potential mediator of p53 tumor suppression. Cell 1993; 75: 817-25.), the 5′ flanking region of the p21 gene comprising p53 recognition sites at −2.2 kb and −1.4 kb (that is to say, the p53 target gene promoter sequence):

(SEQ ID NO:6)

AAGCTTCCCAGGAACATGCTTGGGCAGCAGGCTGTGGCTCTGATTGGCTT
TCTGGCCGTCAGGAACATGTCCCAACATGTTGAGCTCTGGCATACAAGAG
GCTGGTGGCTATTTTGTCCTTGGGCTGCCTGTTTTCAGGTGAGGAAGGGG
ATGGTAGGAGACAGGAGACCTCTAAAGACCCCAGGTAAACCTTAGCCTGT
TACTCTGAACAGGGTATGTGATCTGCCAGCAGATCCTTGCGACAGGGCTG
GGATCTGATGCATGTGTGCTTGTGTGAGTGTGTGCTGGGAGTCAGATTCT
GTGTGTGACTTTTAACAGCCTGCTCCCTTGCCTTTTTCAGGGCAGAAGTC
CTCCCTTAGAGTGTGTCTGGGTACACATTCAAGTGCATGGTTGCAAACTT
TTTTTTTTAAAGCACTGAATAGTACTAGACACTTAGTAGGTACTTAAGAA
ATATTGAATGTCGTGGTGGTGGTGAGCTAGAAGTTATAAAAAAAATTCTT
TCCCAAAAACAACAACAAAAAGAATTATTTCATTGTGAAGCTCAGTACCA
CAAAAATTTAAATAATTCATTACAAGCCTTTATTAAAAAAAATTTTCTCC
CCAAAGTAAACAGACAGACAATGTCTAGTCTATTTGAAATGCCTGAAAGC
AGAGGGGCTTCAAGGCAGTGGGAGAAGGTGCCTGTCCTCTGCTGGACATT
TGACAACCAGCCCTTTGGATGGTTTGGATGTATAGGAGCGAAGGTGCAGA
CAGCAGTGGGGCTTAGAGTGGGGTCCTGAGGCTGTGCCGTGGCCTTTCTG
GGGTTTAGCCACAATCCTGGCCTGACTCCAGGGCGAGGCAGGCCAAGGGG
GTCTGCTACTGTGTCCTCCCACCCCTACCTGGGCTCCCATCCCCACAGCA
GAGGAGAAAGAAGCCTGTCCTCCCCGAGGTCAGCTGCGTTAGAGGAAGAA
GACTGGGCATGTCTGGGCAGAGATTTCCAGACTCTGAGCAGCCTGAGATG
TCAGTAATTGTAGCTGCTCCAAGCCTGGGTTCTGTTTTTTAGTGGGATTT
CTGTTCAGATGAACAATCCATCCTCTGCAATTTTTTAAAAGCAAAACTGC
AAATGTTTCAGGCACAGAAAGGAGGCAAAGGTGAAGTCCAGGGGAGGTCA
GGGGTGTGAGGTAGATGGGAGCGGATAGACACATCACTCATTTCTGTGTC
TGTCAGAAGAACCAGTAGACACTTCCAGAATTGTCCTTTATTTATGTCAT
CTCCATAAACCATCTGCAAATGAGGGTTATTTGGCATTTTTGTCATTTTG
GAGCCACAGAAATAAAGGATGACAAGCAGAGAGCCCCGGGCAGGAGGCAA
AAGTCCTGTGTTCCAACTATAGTCATTTCTTTGCTGCATGATCTGAGTTA
GGTCACCAGACTTCTCTGAGCCCCAGTTTCCCCAGCAGTGTATACGGGCT
ATGTGGGGAGTATTCAGGAGACAGACAACTCACTCGTCAAATCCTCCCCT
TCCTGGCCAACAAAGCTGCTGCAACCACAGGGATTTCTTCTGTTCAGGTG
AGTGTAGGGTGTAGGGAGATTGGTTCAATGTCCAATTCTTCTGTTTCCCT
GGAGATCAGGTTGCCCTTTTTTGGTAGTCTCTCCAATTCCCTCCTTCCCG
GAAGCATGTGACAATCAACAACTTTGTATACTTAAGTTCAGTGGACCTCA
ATTTCCTCATCTGTGAAATAAACGGGACTGAAAAATCATTCTGGCCTCAA
GATGCTTTGTTGGGGTGTCTAGGTGCTCCAGGTGCTTCTGGGAGAGGTGA
CCTAGTGAGGGATCAGTGGGAATAGAGGTGATATTGTGGGGCTTTTCTGG
AAATTGCAGAGAGGTGCATCGTTTTTATAATTTATGAATTTTTATGTATT
AATGTCATCCTCCTGATCTTTTCAGCTGCATTGGGTAAATCCTTGCCTGC
CAGAGTGGGTCAGCGGTGAGCCAGAAAGGGGGCTCATTCTAACAGTGCTG
TGTCCTCCTGGAGAGTGCCAACTCATTCTCCAAGTAAAAAAAGCCAGATT
TGTGGCTCACTTCGTGGGGAAATGTGTCCAGCGCACCAACGCAGGCGAGG
GACTGGGGGAGGAGGGAAGTGCCCTCCTGCAGCACGCGAGGTTCCGGGAC
CGGCTGGCCTGCTGGAACTCGGCCAGGCTCAGCTGGCTCGGCGCTGGGCA
GCCAGGAGCCTGGGCCCCGGGGAGGGCGGTCCCGGGCGGCGCGGTGGGCC
GAGCGCGGGTCCCGCCTCCTTGAGGCGGGCCCGGGCGGGGCGGTTGTATA
TCAGGGCCGCGCTGAGCTGCGCCAGCTGAGGTGTGAGCAGCTGCCGAAGT
CAGTTCCTTGTGGAAGCTT.

The excised 5′ flanking region of the p21 gene was inserted into the multicloning site of the pGL3 basic vector (Promega, pGL3-Basic (Cat.#E1751)) comprising the luciferase gene sequence and the SV40-derived polyadenylation signal to construct the luciferase expression plasmid pLS. The base sequence of the plasmid pLS is shown below.

(SEQ ID NO:7)

GGTACCGAGCTCTTACGCGTGCTAGCCCGGGCTCGAGATCTGCGATCTAA
GTAAGCTTCCCAGGAACATGCTTGGGCAGCAGGCTGTGGCTCTGATTGGC
TTTCTGGCCGTCAGGAACATGTCCCAACATGTTGAGCTCTGGCATAGAAG
AGGCTGGTGGCTATTTTGTCCTTGGGCTGCCTGTTTTCAGGTGAGGAAGG
GGATGGTAGGAGACAGGAGACCTCTAAAGACCCCAGGTAAACCTTAGCCT
GTTACTCTGAACAGGGTATGTGATCTGCCAGCAGATCCTTGCGACAGGGC
TGGGATCTGATGCATGTGTGCTTGTGTGAGTGTGTGCTGGGAGTCAGATT
CTGTGTGTGACTTTTAACAGCCTGCTCCCTTGCCTTTTTCAGGGCAGAAG
TCCTCCCTTAGAGTGTGTCTGGGTACACATTCAAGTGCATGGTTGCAAAC
TTTTTTTTTTAAAGCACTGAATAGTACTAGACACTTAGTAGGTACTTAAG
AAATATTGAATGTCGTGGTGGTGGTGAGCTAGAAGTTATAAAAAAAATTC
TTTCCCAAAAACAACAACAAAAAGAATTATTTCATTGTGAAGCTCAGTAC
CACAAAAATTTAAATAATTCATTACAAGCCTTTATTAAAAAAAATTTTCT
CCCCAAAGTAAACAGACAGACAATGTCTAGTCTATTTGAAATGCCTGAAA
GCAGAGGGGCTTCAAGGCAGTGGGAGAAGGTGCCTGTCCTCTGCTGGACA
TTTGACAACCAGCCCTTTGGATGGTTTGGATGTATAGGAGCGAAGGTGCA
GACAGCAGTGGGGCTTAGAGTGGGGTCCTGAGGCTGTGCCGTGGCCTTTC
TGGGGTTTAGCCACAATCCTGGCCTGACTCCAGGGCGAGGCAGGCCAAGG
GGGTCTGCTACTGTGTCCTCCCACCCCTACCTGGGCTCCCATCCCCACAG
CAGAGGAGAAAGAAGCCTGTCCTCCCCGAGGTCAGCTGCGTTAGAGGAAG
AAGACTGGGCATGTCTGGGCAGAGATTTCCAGACTCTGAGCAGCCTGAGA
TGTCAGTAATTGTAGCTGCTCCAAGCCTGGGTTCTGTTTTTTAGTGGGAT
TTCTGTTCAGATGAACAATCCATCCTCTGCAATTTTTTAAAAGCAAAACT
GCAAATGTTTCAGGCACAGAAAGGAGGCAAAGGTGAAGTCCAGGGGAGGT
CAGGGGTGTGAGGTAGATGGGAGCGGATAGACACATCACTCATTTCTGTG
TCTGTCAGAAGAACCAGTAGACACTTCCAGAATTGTCCTTTATTTATGTC
ATCTCCATAAACCATCTGCAAATGAGGGTTATTTGGCATTTTTGTCATTT
TGGAGCCACAGAAATAAAGGATGACAAGCAGAGAGCCCCGGGCAGGAGGC
AAAAGTCCTGTGTTCCAACTATAGTCATTTCTTTGCTGCATGATCTGAGT
TAGGTCACCAGACTTCTCTGAGCCCCAGTTTCCCCAGCAGTGTATACGGG
CTATGTGGGGAGTATTCAGGAGACAGACAACTCACTCGTCAAATCCTCCC
CTTCCTGGCCAACAAAGCTGCTGCAACCACAGGGATTTCTTCTGTTCAGG
TGAGTGTAGGGTGTAGGGAGATTGGTTCAATGTCCAATTCTTCTGTTTCC
CTGGAGATCAGGTTGCCCTTTTTTGGTAGTCTCTCCAATTCCCTCCTTCC
CGGAAGCATGTGACAATCAACAACTTTGTATACTTAAGTTCAGTGGACCT
CAATTTCCTCATCTGTGAAATAAACGGGACTGAAAAATCATTCTGGCCTC
AAGATGCTTTGTTGGGGTGTCTAGGTGCTCCAGGTGCTTCTGGGAGAGGT
GACCTAGTGAGGGATCAGTGGGAATAGAGGTGATATTGTGGGGCTTTTCT
GGAAATTGCAGAGAGGTGCATCGTTTTTATAATTTATGAATTTTTATGTA
TTAATGTCATCCTCCTGATCTTTTCAGCTGCATTGGGTAAATCCTTGCCT
GCCAGAGTGGGTCAGCGGTGAGCCAGAAAGGGGGCTCATTCTAACAGTGC
TGTGTCCTCCTGGAGAGTGCCAACTCATTCTCCAAGTAAAAAAAGCCAGA
TTTGTGGCTCACTTCGTGGGGAAATGTGTCCAGCGCACCAACGCAGGCGA
GGGACTGGGGGAGGAGGGAAGTGCCCTCCTGCAGCACGCGAGGTTCCGGG
ACCGGCTGGCCTGCTGGAACTCGGCCAGGCTCAGCTGGCTCGGCGCTGGG
CAGCCAGGAGCCTGGGCCCCGGGGAGGGCGGTCCCGGGCGGCGCGGTGGG
CCGAGCGCGGGTCCCGCCTCCTTGAGGCGGGCCCGGGCGGGGCGGTTGTA
TATCAGGGCCGCGCTGAGCTGCGCCAGCTGAGGTGTGAGCAGCTGCCGAA
GTCAGTTCCTTGTGGAAGCTTGGCATTCCGGTACTGTTGGTAAAGCCACC
ATGGAAGACGCCAAAAACATAAAGAAAGGCCCGGCGCCATTCTATCCGCT
GGAAGATGGAACCGCTGGAGAGCAACTGCATAAGGCTATGAAGAGATACG
CCCTGGTTCCTGGAACAATTGCTTTTACAGATGCACATATCGAGGTGGAC
ATCACTTACGCTGAGTACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGGGCATTTCGCAGCCTACCGTGGTGTTCGTTTCCAAAAAGGGGTTGC
AAAAAATTTTGAACGTGCAAAAAAAGCTCCCAATCATCCAAAAAATTATT
ATCATGGATTCTAAAACGGATTACCAGGGATTTCAGTCGATGTACACGTT
CGTCACATCTCATCTACCTCCCGGTTTTAATGAATACGATTTTGTGCCAG
AGTCCTTCGATAGGGACAAGACAATTGCACTGATCATGAACTCCTCTGGA
TCTACTGGTCTGCCTAAAGGTGTCGCTCTGCCTCATAGAACTGCCTGCGT
GAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTCTGAGGAGCCTTCAGGATTACAAGATTCAAA
GTGCGCTGCTGGTGCCAACCCTATTCTCCTTCTTCGCCAAAAGCACTCTG
ATTGACAAATACGATTTATCTAATTTACACGAAATTGCTTCTGGTGGCGC
TCCCCTCTCTAAGGAAGTCGGGGAAGCGGTTGCCAAGAGGTTCCATCTGC
CAGGTATCAGGCAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAAAGAGGCGAACTGTGTGTGAGAGGTCCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TCGTTGACCGCCTGAAGTCTCTGATTAAGTACAAAGGCTATCAGGTGGCT
CCCGCTGAATTGGAATCCATCTTGCTCCAACACCCCAACATCTTCGACGC
AGGTGTCGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGATCGCCGTG
TAATTCTAGAGTCGGGGCGGCCGGCCGCTTCGAGCAGACATGATAAGATA
CATTGATGAGTTTGGACAAACCACAACTAGAATGCAGTGAAAAAAATGCT
TTATTTGTGAAATTTGTGATGCTATTGCTTTATTTGTAACCATTATAAGC
TGCAATAAACAAGTTAACAACAACAATTGCATTCATTTTATGTTTCAGGT
TCAGGGGGAGGTGTGGGAGGTTTTTTAAAGCAAGTAAAACCTCTACAAAT
GTGGTAAAATCGATAAGGATCCGTCGACCGATGCCCTTGAGAGCCTTCAA
CCCAGTCAGCTCCTTCCGGTGGGCGCGGGGCATGACTATCGTCGCCGCAC
TTATGACTGTCTTCTTTATCATGCAACTCGTAGGACAGGTGCCGGCAGCG
CTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAA
TCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAAC
CCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTC
AGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCA
ACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTG
GCCTAACTACGGCTACACTAGAAGAACAGTATTTGGTATCTGCGCTCTGC
TGAAGCCAGTTACCTTCGGAAAAAGAGTTGGTAGCTCTTGATCCGGCAAA
CAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGT
CTGACGCTCAGTGGAACGAAAACTCACGTTAAGGGATTTTGGTCATGAGA
TTATCAAAAAGGATCTTCACCTAGATCCTTTTAAATTAAAAATGAAGTTT
TAAATCAATCTAAAGTATATATGAGTAAACTTGGTCTGACAGTTACCAAT
GCTTAATCAGTGAGGCACCTATCTCAGCGATCTGTCTATTTCGTTCATCC
ATAGTTGCCTGACTCCCCGTCGTGTAGATAACTACGATACGGGAGGGCTT
ACCATCTGGCCCCAGTGCTGCAATGATACCGCGAGACCCACGCTCACCGG
CTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTG
CCATTGCTACAGGCATCGTGGTGTCACGCTCGTCGTTTGGTATGGCTTCA
TTCAGCTCCGGTTCCCAACGATCAAGGCGAGTTACATGATCCCCCATGTT
GTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCT
CTTACTGTCATGCCATCCGTAAGATGCTTTTCTGTGACTGGTGAGTACTC
AACCAAGTCATTCTGAGAATAGTGTATGCGGCGACCGAGTTGCTCTTGCC
CGGCGTCAATACGGGATAATACCGCGCCACATAGCAGAACTTTAAAAGTG
CTCATCATTGGAAAACGTTCTTCGGGGCGAAAACTCTCAAGGATCTTACC
GCTGTTGAGATCCAGTTCGATGTAACCCACTCGTGCACCCAACTGATCTT
CAGCATCTTTTACTTTCACCAGCGTTTCTGGGTGAGCAAAAACAGGAAGG
CAAAATGCCGCAAAAAAGGGAATAAGGGCGACACGGAAATGTTGAATACT
CATACTCTTCCTTTTTCAATATTATTGAAGCATTTATCAGGGTTATTGTC
TCATGAGCGGATACATATTTGAATGTATTTAGAAAAATAAACAAATAGGG
GTTCCGCGCACATTTCCCCGAAAAGTGCCACCTGACGCGCCCTGTAGCGG
CGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATT
AGGGTGATGGTTCACGTAGTGGGCCATCGCCCTGATAGACGGTTTTTCGC
CCTTTGACGTTGGAGTCCACGTTCTTTAATAGTGGACTCTTGTTCCAAAC
TGGAACAACACTCAACCCTATCTCGGTCTATTCTTTTGATTTATAAGGGA
TTTTGCCGATTTCGGCCTATTGGTTAAAAAATGAGCTGATTTAACAAAAA
TTTAACGCGAATTTTAACAAAATATTAACGCTTACAATTTGCCATTCGCC
ATTCAGGCTGCGCAACTGTTGGGAAGGGCGATCGGTGCGGGCCTCTTCGC
TATTACGCCAGCCCAAGCTACCATGATAAGTAAGTAATATTAAGGTACGG
GAGGTACTTGGAGCGGCCGCAATAAAATATCTTTATTTTCATTACATCTG
TGTGTTGGTTTTTTGTGTGAATCGATAGTACTAACATACGCTCTCCATCA
AAACAAAACGAAACAAAACAAACTAGCAAAATAGGCTGTCCCCAGTGCAA
GTGCAGGTGCCAGAACATTTCTCTATCGATA

In the base sequence, the 5′ flanking region of the p21 gene is the region represented by the following sequence:

(SEQ ID NO:8)

AAGCTTCCCAGGAACATGCTTGGGCAGCAGGCTGTGGCTCTGATTGGCTT
TCTGGCCGTCAGGAACATGTCCCAACATGTTGAGCTCTGGCATAGAAGAG
GCTGGTGGCTATTTTGTCCTTGGGCTGCCTGTTTTCAGGTGAGGAAGGGG
ATGGTAGGAGACAGGAGACCTCTAAAGACCCCAGGTAAACCTTAGCCTGT
TACTCTGAACAGGGTATGTGATCTGCCAGCAGATCCTTGCGACAGGGCTG
GGATCTGATGCATGTGTGCTTGTGTGAGTGTGTGCTGGGAGTCAGATTCT
GTGTGTGACTTTTAACAGCCTGCTCCCTTGCCTTTTTCAGGGCAGAAGTC
CTCCCTTAGAGTGTGTCTGGGTACACATTCAAGTGCATGGTTGCAAACTT
TTTTTTTTAAAGCACTGAATAGTACTAGACACTTAGTAGGTACTTAAGAA
ATATTGAATGTCGTGGTGGTGGTGAGCTAGAAGTTATAAAAAAAATTCTT
TCCCAAAAACAACAACAAAAAGAATTATTTCATTGTGAAGCTCAGTACCA
CAAAAATTTAAATAATTCATTACAAGCCTTTATTAAAAAAAATTTTCTCC
CCAAAGTAAACAGACAGACAATGTCTAGTCTATTTGAAATGCCTGAAAGC
AGAGGGGCTTCAAGGCAGTGGGAGAAGGTGCCTGTCCTCTGCTGGACATT
TGACAACCAGCCCTTTGGATGGTTTGGATGTATAGGAGCGAAGGTGCAGA
CAGCAGTGGGGCTTAGAGTGGGGTCCTGAGGCTGTGCCGTGGCCTTTCTG
GGGTTTAGCCACAATCCTGGCCTGACTCCAGGGCGAGGCAGGCCAAGGGG
GTCTGCTACTGTGTCCTCCCACCCCTACCTGGGCTCCCATCCCCACAGCA
GAGGAGAAAGAAGCCTGTCCTCCCCGAGGTCAGCTGCGTTAGAGGAAGAA
GACTGGGCATGTCTGGGCAGAGATTTCCAGACTCTGAGCAGCCTGAGATG
TCAGTAATTGTAGCTGCTCCAAGCCTGGGTTCTGTTTTTTAGTGGGATTT
CTGTTCAGATGAACAATCCATCCTCTGCAATTTTTTAAAAGCAAAACTGC
AAATGTTTCAGGCACAGAAAGGAGGCAAAGGTGAAGTCCAGGGGAGGTCA
GGGGTGTGAGGTAGATGGGAGCGGATAGACACATCACTCATTTCTGTGTC
TGTCAGAAGAACCAGTAGACACTTCCAGAATTGTCCTTTATTTATGTCAT
CTCCATAAACCATCTGCAAATGAGGGTTATTTGGCATTTTTGTCATTTTG
GAGCCACAGAAATAAAGGATGACAAGCAGAGAGCCCCGGGCAGGAGGCAA
AAGTCCTGTGTTCCAACTATAGTCATTTCTTTGCTGCATGATCTGAGTTA
GGTCACCAGACTTCTCTGAGCCCCAGTTTCCCCAGCAGTGTATACGGGCT
ATGTGGGGAGTATTCAGGAGACAGACAACTCACTCGTCAAATCCTCCCCT
TCCTGGCCAACAAAGCTGCTGCAACCACAGGGATTTCTTCTGTTCAGGTG
AGTGTAGGGTGTAGGGAGATTGGTTCAATGTCCAATTCTTCTGTTTCCCT
GGAGATCAGGTTGCCCTTTTTTGGTAGTCTCTCCAATTCCCTCCTTCCCG
GAAGCATGTGACAATCAACAACTTTGTATACTTAAGTTCAGTGGACCTCA
ATTTCCTCATCTGTGAAATAAACGGGACTGAAAAATCATTCTGGCCTCAA
GATGCTTTGTTGGGGTGTCTAGGTGCTCCAGGTGCTTCTGGGAGAGGTGA
CCTAGTGAGGGATCAGTGGGAATAGAGGTGATATTGTGGGGCTTTTCTGG
AAATTGCAGAGAGGTGCATCGTTTTTATAATTTATGAATTTTTATGTATT
AATGTCATCCTCCTGATCTTTTCAGCTGCATTGGGTAAATCCTTGCCTGC
CAGAGTGGGTCAGCGGTGAGCCAGAAAGGGGGCTCATTCTAACAGTGCTG
TGTCCTCCTGGAGAGTGCCAACTCATTCTCCAAGTAAAAAAAGCCAGATT
TGTGGCTCACTTCGTGGGGAAATGTGTCCAGCGCACCAACGCAGGCGAGG
GACTGGGGGAGGAGGGAAGTGCCCTCCTGCAGCACGCGAGGTTCCGGGAC
CGGCTGGCCTGCTGGAACTCGGCCAGGCTCAGCTGGCTCGGCGCTGGGCA
GCCAGGAGCCTGGGCCCCGGGGAGGGCGGTCCCGGGCGGCGCGGTGGGCC
GAGCGCGGGTCCCGCCTCCTTGAGGCGGGCCCGGGCGGGGCGGTTGTATA
TCAGGGCCGCGCTGAGCTGCGCCAGCTGAGGTGTGAGCAGCTGCCGAAGT
CAGTTCCTTGTGGAAGCTT,

the luciferase coding sequence is the region represented by the following sequence:

(SEQ ID NO:9)

ATGGAAGACGCCAAAAACATAAAGAAAGGCCCGGCGCCATTCTATCCGCT
GGAAGATGGAACCGCTGGAGAGCAACTGCATAAGGCTATGAAGAGATACG
CCCTGGTTCCTGGAACAATTGCTTTTACAGATGCACATATCGAGGTGGAC
ATCACTTACGCTGAGTACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGGGCATTTCGCAGCCTACCGTGGTGTTCGTTTCCAAAAAGGGGTTGC
AAAAAATTTTGAACGTGCAAAAAAAGCTCCCAATCATCCAAAAAATTATT
ATCATGGATTCTAAAACGGATTACCAGGGATTTCAGTCGATGTACACGTT
CGTCACATCTCATCTACCTCCCGGTTTTAATGAATACGATTTTGTGCCAG
AGTCCTTCGATAGGGACAAGACAATTGCACTGATCATGAACTCCTCTGGA
TCTACTGGTCTGCCTAAAGGTGTCGCTCTGCCTCATAGAACTGCCTGCGT
GAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTCTGAGGAGCCTTCAGGATTACAAGATTCAAA
GTGCGCTGCTGGTGCCAACCCTATTCTCCTTCTTCGCCAAAAGCACTCTG
ATTGACAAATACGATTTATCTAATTTACACGAAATTGCTTCTGGTGGCGC
TCCCCTCTCTAAGGAAGTCGGGGAAGCGGTTGCCAAGAGGTTCCATCTGC
CAGGTATCAGGCAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAAAGAGGCGAACTGTGTGTGAGAGGTCCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TCGTTGACCGCCTGAAGTCTCTGATTAAGTACAAAGGCTATCAGGTGGCT
CCCGCTGAATTGGAATCCATCTTGCTCCAACACCCCAACATCTTCGACGC
AGGTGTCGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGATCGCCGTG
TAA,
and

the SV40-derived polyadenylation signal is the region represented by the following sequence:

(SEQ ID NO:10)

CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGGTGTGGGAGGTTTTTTAAAGCAAG
TAAAACCTCTACAAATGTGGTA.

From the constructed plasmid pLS, a 4.3 kb XhoI/BamH1 fragment comprising the 5′ flanking region of the p21 gene, the luciferase coding sequence and the SV40-derived polyadenylation signal was excised and blunted. The obtained fragment was inserted between the two ITRs of pAAV-MCS having a Left-ITR and a Right-ITR (STRATAGENE AAV Helper-Free System Cat#240071) digested with NotI and blunted using Blunting kit (Takara) to obtain the plasmid pAAV-PLS. The base sequence of the plasmid pAAV-PLS is shown below.

(SEQ ID NO:11)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCTCG
AGATCTGCGATCTAAGTAAGCTTCCCAGGAACATGCTTGGGCAGCAGGCT
GTGGCTCTGATTGGCTTTCTGGCCGTCAGGAACATGTCCCAACATGTTGA
GCTCTGGCATAGAAGAGGCTGGTGGCTATTTTGTCCTTGGGCTGCCTGTT
TTCAGGTGAGGAAGGGGATGGTAGGAGACAGGAGACCTCTAAAGACCCCA
GGTAAACCTTAGCCTGTTACTCTGAACAGGGTATGTGATCTGCCAGCAGA
TCCTTGCGACAGGGCTGGGATCTGATGCATGTGTGCTTGTGTGAGTGTGT
GCTGGGAGTCAGATTCTGTGTGTGACTTTTAACAGCCTGCTCCCTTGCCT
TTTTCAGGGCAGAAGTCCTCCCTTAGAGTGTGTCTGGGTACACATTCAAG
TGCATGGTTGCAAACTTTTTTTTTTAAAGCACTGAATAGTACTAGACACT
TAGTAGGTACTTAAGAAATATTGAATGTCGTGGTGGTGGTGAGCTAGAAG
TTATAAAAAAAATTCTTTCCCAAAAACAACAACAAAAAGAATTATTTCAT
TGTGAAGCTCAGTACCACAAAAATTTAAATAATTCATTACAAGCCTTTAT
TAAAAAAAATTTTCTCCCCAAAGTAAACAGACAGACAATGTCTAGTCTAT
TTGAAATGCCTGAAAGCAGAGGGGCTTCAAGGCAGTGGGAGAAGGTGCCT
GTCCTCTGCTGGACATTTGACAACCAGCCCTTTGGATGGTTTGGATGTAT
AGGAGCGAAGGTGCAGACAGCAGTGGGGCTTAGAGTGGGGTCCTGAGGCT
GTGCCGTGGCCTTTCTGGGGTTTAGCCACAATCCTGGCCTGACTCCAGGG
CGAGGCAGGCCAAGGGGGTCTGCTACTGTGTCCTCCCACCCCTACCTGGG
CTCCCATCCCCACAGCAGAGGAGAAAGAAGCCTGTCCTCCCCGAGGTCAG
CTGCGTTAGAGGAAGAAGACTGGGCATGTCTGGGCAGAGATTTCCAGACT
CTGAGCAGCCTGAGATGTCAGTAATTGTAGCTGCTCCAAGCCTGGGTTCT
GTTTTTTAGTGGGATTTCTGTTCAGATGAACAATCCATCCTCTGCAATTT
TTTAAAAGCAAAACTGCAAATGTTTCAGGCACAGAAAGGAGGCAAAGGTG
AAGTCCAGGGGAGGTCAGGGGTGTGAGGTAGATGGGAGCGGATAGACACA
TCACTCATTTCTGTGTCTGTCAGAAGAACCAGTAGACACTTCCAGAATTG
TCCTTTATTTATGTCATCTCCATAAACCATCTGCAAATGAGGGTTATTTG
GCATTTTTGTCATTTTGGAGCCACAGAAATAAAGGATGACAAGCAGAGAG
CCCCGGGCAGGAGGCAAAAGTCCTGTGTTCCAACTATAGTCATTTCTTTG
CTGCATGATCTGAGTTAGGTCACCAGACTTCTCTGAGCCCCAGTTTCCCC
AGCAGTGTATACGGGCTATGTGGGGAGTATTCAGGAGACAGACAACTCAC
TCGTCAAATCCTCCCCTTCCTGGCCAACAAAGCTGCTGCAACCACAGGGA
TTTCTTCTGTTCAGGTGAGTGTAGGGTGTAGGGAGATTGGTTCAATGTCC
AATTCTTCTGTTTCCCTGGAGATCAGGTTGCCCTTTTTTGGTAGTCTCTC
CAATTCCCTCCTTCCCGGAAGCATGTGACAATCAACAACTTTGTATACTT
AAGTTCAGTGGACCTCAATTTCCTCATCTGTGAAATAAACGGGACTGAAA
AATCATTCTGGCCTCAAGATGCTTTGTTGGGGTGTCTAGGTGCTCCAGGT
GCTTCTGGGAGAGGTGACCTAGTGAGGGATCAGTGGGAATAGAGGTGATA
TTGTGGGGCTTTTCTGGAAATTGCAGAGAGGTGCATCGTTTTTATAATTT
ATGAATTTTTATGTATTAATGTCATCCTCCTGATCTTTTCAGCTGCATTG
GGTAAATCCTTGCCTGCCAGAGTGGGTCAGCGGTGAGCCAGAAAGGGGGC
TCATTCTAACAGTGCTGTGTCCTCCTGGAGAGTGCCAACTCATTCTCCAA
GTAAAAAAAGCCAGATTTGTGGCTCACTTCGTGGGGAAATGTGTCCAGCG
CACCAACGCAGGCGAGGGACTGGGGGAGGAGGGAAGTGCCCTCCTGCAGC
ACGCGAGGTTCCGGGACCGGCTGGCCTGCTGGAACTCGGCCAGGCTCAGC
TGGCTCGGCGCTGGGCAGCCAGGAGCCTGGGCCCCGGGGAGGGCGGTCCC
GGGCGGCGCGGTGGGCCGAGCGCGGGTCCCGCCTCCTTGAGGCGGGCCCG
GGCGGGGCGGTTGTATATCAGGGCCGCGCTGAGCTGCGCCAGCTGAGGTG
TGAGCAGCTGCCGAAGTCAGTTCCTTGTGCAAGCTTGGCATTCCGGTACT
GTTGGTAAAGCCACCATGGAAGACGCCAAAAACATAAAGAAAGGCCCGGC
GCCATTCTATCCGCTGGAAGATGGAACCGCTGGAGAGCAACTGCATAAGG
CTATGAAGAGATACGCCCTGGTTCCTGGAACAATTGCTTTTACAGATGCA
CATATCGAGGTGGACATCACTTACGCTGAGTACTTCGAAATGTCCGTTCG
GTTGGCAGAAGCTATGAAACGATATGGGCTGAATACAAATCACAGAATCG
TCGTATGCAGTGAAAACTCTCTTCAATTCTTTATGCCGGTGTTGGGCGCG
TTATTTATCGGAGTTGCAGTTGCGCCCGCGAACGACATTTATAATGAACG
TGAATTGCTCAACAGTATGGGCATTTCGCAGCCTACCGTGGTGTTCGTTT
CCAAAAAGGGGTTGCAAAAAATTTTGAACGTGCAAAAAAAGCTCCCAATC
ATCCAAAAAATTATTATCATGGATTCTAAAACGGATTACCAGGGATTTCA
GTCGATGTACACGTTCGTCACATCTCATCTACCTCCCGGTTTTAATGAAT
ACGATTTTGTGCCAGAGTCCTTCGATAGGGACAAGACAATTGCACTGATC
ATGAACTCCTCTGGATCTACTGGTCTGCCTAAAGGTGTCGCTCTGCCTCA
TAGAACTGCCTGCGTGAGATTCTCGCATGCCAGAGATCCTATTTTTGGCA
ATCAAATCATTCCGGATACTGCGATTTTAAGTGTTGTTCCATTCCATCAC
GGTTTTGGAATGTTTACTACACTCGGATATTTGATATGTGGATTTCGAGT
CGTCTTAATGTATAGATTTGAAGAAGAGCTGTTTCTGAGGAGCCTTCAGG
ATTACAAGATTCAAAGTGCGCTGCTGGTGCCAACCCTATTCTCCTTCTTC
GCCAAAAGCACTCTGATTGACAAATACGATTTATCTAATTTACACGAAAT
TGCTTCTGGTGGCGCTCCCCTCTCTAAGGAAGTCGGGGAAGCGGTTGCCA
AGAGGTTCCATCTGCCAGGTATCAGGCAAGGATATGGGCTCACTGAGACT
ACATCAGCTATTCTGATTACACCCGAGGGGGATGATAAACCGGGCGCGGT
CGGTAAAGTTGTTCCATTTTTTGAAGCGAAGGTTGTGGATCTGGATACCG
GGAAAACGCTGGGCGTTAATCAAAGAGGCGAACTGTGTGTGAGAGGTCCT
ATGATTATGTCCGGTTATGTAAACAATCCGGAAGCGACCAACGCCTTGAT
TGACAAGGATGGATGGCTACATTCTGGAGACATAGCTTACTGGGACGAAG
ACGAACACTTCTTCATCGTTGACCGCCTGAAGTCTCTGATTAAGTACAAA
GGCTATCAGGTGGCTCCCGCTGAATTGGAATCCATCTTGCTCCAACACCC
CAACATCTTCGACGCAGGTGTCGCAGGTCTTCCCGACGATGACGCCGGTG
AACTTCCCGCCGCCGTTGTTGTTTTGGAGCACGGAAAGACGATGACGGAA
AAAGAGATCGTGGATTACGTCGCCAGTCAAGTAACAACCGCGAAAAAGTT
GCGCGGAGGAGTTGTGTTTGTGGACGAAGTACCGAAAGGTCTTACCGGAA
AACTCGACGCAAGAAAAATCAGAGAGATCCTCATAAAGGCCAAGAAGGGC
GGAAAGATCGCCGTGTAATTCTAGAGTCGGGGCGGCCGGCCGCTTCGAGC
AGACATGATAAGATACATTGATGAGTTTGGACAAACCACAACTAGAATGC
AGTGAAAAAAATGCTTTATTTGTGAAATTTGTGATGCTATTGCTTTATTT
GTAACCATTATAAGCTGCAATAAACAAGTTAACAACAACAATTGCATTCA
TTTTATGTTTCAGGTTCAGGGGGAGGTGTGGGAGGTTTTTTAAAGCAAGT
AAAACCTCTACAAATGTGGTAAAATCGATAAGGATCGGCCGCAGGAACCC
CTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCT
CAGTGAGCGAGCGAGCGCGCAGCTGCCTGCAGGGGCGCCTGATGCGGTAT
TTTCTCCTTACGCATCTGTGCGGTATTTCACACCGCATACGTCAAAGCAA
CCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCA
CCTCGACCCCAAAAAACTTGATTTGGGTGATGGTTCACGTAGTGGGCCAT
CGCCCTGATAGACGGTTTTTCGCCCTTTGACGTTGGAGTCCACGTTCTTT
AATAGTGGACTCTTGTTCCAAACTGGAACAACACTCAACCCTATCTCGGG
CTATTCTTTTGATTTATAAGGGATTTTGCCGATTTCGGCCTATTGGTTAA
AAAATGAGCTGATTTAACAAAAATTTAACGCGAATTTTAACAAAATATTA
ACGTTTACAATTTTATGGTGCACTCTCAGTACAATCTGCTCTGATGCCGC
ATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
ACGAAAGGGCCTCGTGATACGCCTATTTTTATAGGTTAATGTCATGATAA
TAATGGTTTCTTAGACGTCAGGTGGCACTTTTCGGGGAAATGTGCGCGGA
ACCCCTATTTGTTTATTTTTCTAAATACATTCAAATATGTATCCGCTCAT
GAGACAATAACCCTGATAAATGCTTCAATAATATTGAAAAAGGAAGAGTA
TGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGC
TGAAGATCAGTTGGGTGCACGAGTGGGTTACATCGAACTGGATCTCAACA
GCGGTAAGATCCTTGAGAGTTTTCGCCCCGAAGAACGTTTTCCAATGATG
AGCACTTTTAAAGTTCTGCTATGTGGCGCGGTATTATCCCGTATTGACGC
CGGGCAAGAGCAACTCGGTCGCCGCATACACTATTCTCAGAATGACTTGG
TTGAGTACTCACCAGTCACAGAAAAGCATCTTACGGATGGCATGACAGTA
AGAGAATTATGCAGTGCTGCCATAACCATGAGTGATAACACTGCGGCCAA
CTTACTTCTGACAACGATCGGAGGACCGAAGGAGCTAACCGCTTTTTTGC
ACAACATGGGGGATCATGTAACTCGCCTTGATCGTTGGGAACCGGAGCTG
AATGAAGCCATACCAAACGACGAGCGTGACACCACGATGCCTGTAGCAAT
GGCAACAACGTTGCGCAAACTATTAACTGGCGAACTACTTACTCTAGCTT
CCCGGCAACAATTAATAGACTGGATGGAGGCGGATAAAGTTGCAGGACCA
CTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATG
GTAAGCCCTCCCGTATCGTAGTTATCTACACGACGGGGAGTCAGGCAACT
ATGGATGAACGAAATAGACAGATCGCTGAGATAGGTGCCTCACTGATTAA
GCATTGGTAACTGTCAGACCAAGTTTACTCATATATACTTTAGATTGATT
TAAAACTTCATTTTTAATTTAAAAGGATCTAGGTGAAGATCCTTTTTGAT
AATCTCATGACCAAAATCCCTTAACGTGAGTTTTCGTTCCACTGAGCGTC
AGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTT
CAGCAGAGCGCAGATACCAAATACTGTCCTTCTAGTGTAGCCGTAGTTAG
GCCACCACTTCAAGAACTCTGTAGCACCGCCTACATACCTCGCTCTGCTA
ATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAA
CGGGGGGTTCGTGCACACAGCCCAGCTTGGAGCGAACGACCTACACCGAA
CTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCC
TGGCCTTTTGCTGGCCTTTTGCTCACATGT

In the base sequence, the Left-ITR is the region represented by the following sequence:

(SEQ ID NO:12)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT,

the 5′ flanking region of the p21 gene is the region represented by the following sequence:

(SEQ ID NO:13)

AAGCTTCCCAGGAACATGCTTGGGCAGCAGGCTGTGGCTCTGATTGGCTT
TCTGGCCGTCAGGAACATGTCCCAACATGTTGAGCTCTGGCATAGAAGAG
GCTGGTGGCTATTTTGTCCTTGGGCTGCCTGTTTTCAGGTGAGGAAGGGG
ATGGTAGGAGACAGGAGACCTCTAAAGACCCCAGGTAAACCTTAGCCTGT
TACTCTGAACAGGGTATGTGATCTGCCAGCAGATCCTTGCGACAGGGCTG
GGATCTGATGCATGTGTGCTTGTGTGAGTGTGTGCTGGGAGTCAGATTCT
GTGTGTGACTTTTAACAGCCTGCTCCCTTGCCTTTTTCAGGGCAGAAGTC
CTCCCTTAGAGTGTGTCTGGGTACACATTCAAGTGCATGGTTGCAAACTT
TTTTTTTTAAAGCACTGAATAGTACTAGACACTTAGTAGGTACTTAAGAA
ATATTGAATGTCGTGGTGGTGGTGAGCTAGAAGTTATAAAAAAAATTCTT
TCCCAAAAACAACAACAAAAAGAATTATTTCATTGTGAAGCTCAGTACCA
CAAAAATTTAAATAATTCATTACAAGCCTTTATTAAAAAAAATTTTCTCC
CCAAAGTAAACAGACAGACAATGTCTAGTCTATTTGAAATGCCTGAAAGC
AGAGGGGCTTCAAGGCAGTGGGAGAAGGTGCCTGTCCTCTGCTGGACATT
TGACAACCAGCCCTTTGGATGGTTTGGATGTATAGGAGCGAAGGTGCAGA
CAGCAGTGGGGCTTAGAGTGGGGTCCTGAGGCTGTGCCGTGGCCTTTCTG
GGGTTTAGCCACAATCCTGGCCTGACTCCAGGGCGAGGCAGGCCAAGGGG
GTCTGCTACTGTGTCCTCCCACCCCTACCTGGGCTCCCATCCCCACAGCA
GAGGAGAAAGAAGCCTGTCCTCCCCGAGGTCAGCTGCGTTAGAGGAAGAA
GACTGGGCATGTCTGGGCAGAGATTTCCAGACTCTGAGCAGCCTGAGATG
TCAGTAATTGTAGCTGCTCCAAGCCTGGGTTCTGTTTTTTAGTGGGATTT
CTGTTCAGATGAACAATCCATCCTCTGCAATTTTTTAAAAGCAAAACTGC
AAATGTTTCAGGCACAGAAAGGAGGCAAAGGTGAAGTCCAGGGGAGGTCA
GGGGTGTGAGGTAGATGGGAGCGGATAGACACATCACTCATTTCTGTGTC
TGTCAGAAGAACCAGTAGACACTTCCAGAATTGTCCTTTATTTATGTCAT
CTCCATAAACCATCTGCAAATGAGGGTTATTTGGCATTTTTGTCATTTTG
GAGCCACAGAAATAAAGGATGACAAGCAGAGAGCCCCGGGCAGGAGGCAA
AAGTCCTGTGTTCCAACTATAGTCATTTCTTTGCTGCATGATCTGAGTTA
GGTCACCAGACTTCTCTGAGCCCCAGTTTCCCCAGCAGTGTATACGGGCT
ATGTGGGGAGTATTCAGGAGACAGACAACTCACTCGTCAAATCCTCCCCT
TCCTGGCCAACAAAGCTGCTGCAACCACAGGGATTTCTTCTGTTCAGGTG
AGTGTAGGGTGTAGGGAGATTGGTTCAATGTCCAATTCTTCTGTTTCCCT
GGAGATCAGGTTGCCCTTTTTTGGTAGTCTCTCCAATTCCCTCCTTCCCG
GAAGCATGTGACAATCAACAACTTTGTATACTTAAGTTCAGTGGACCTCA
ATTTCCTCATCTGTGAAATAAACGGGACTGAAAAATCATTCTGGCCTCAA
GATGCTTTGTTGGGGTGTCTAGGTGCTCCAGGTGCTTCTGGGAGAGGTGA
CCTAGTGAGGGATCAGTGGGAATAGAGGTGATATTGTGGGGCTTTTCTGG
AAATTGCAGAGAGGTGCATCGTTTTTATAATTTATGAATTTTTATGTATT
AATGTCATCCTCCTGATCTTTTCAGCTGCATTGGGTAAATCCTTGCCTGC
CAGAGTGGGTCAGCGGTGAGCCAGAAAGGGGGCTCATTCTAACAGTGCTG
TGTCCTCCTGGAGAGTGCCAACTCATTCTCCAAGTAAAAAAAGCCAGATT
TGTGGCTCACTTCGTGGGGAAATGTGTCCAGCGCACCAACGCAGGCGAGG
GACTGGGGGAGGAGGGAAGTGCCCTCCTGCAGCACGCGAGGTTCCGGGAC
CGGCTGGCCTGCTGGAACTCGGCCAGGCTCAGCTGGCTCGGCGCTGGGCA
GCCAGGAGCCTGGGCCCCGGGGAGGGCGGTCCCGGGCGGCGCGGTGGGCC
GAGCGCGGGTCCCGCCTCCTTGAGGCGGGCCCGGGCGGGGCGGTTGTATA
TCAGGGCCGCGCTGAGCTGCGCCAGCTGAGGTGTGAGCAGCTGCCGAAGT
CAGTTCCTTGTGGAAGCTT,

the luciferase coding sequence is the region represented by the following sequence:

(SEQ ID NO: 14)

ATGGAAGACGCCAAAAACATAAAGAAAGGCCCGGCGCCATTCTATCCGCT
GGAAGATGGAACCGCTGGAGAGCAACTGCATAAGGCTATGAAGAGATACG
CCCTGGTTCCTGGAACAATTGCTTTTACAGATGCACATATCGAGGTGGAC
ATCACTTACGCTGAGTACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTCAATTGCTCAACAG
TATGGGCATTTCGCAGCCTACCCTGGTGTTCGTTTCCAAAAAGGGGTTGC
AAAAAATTTTGAACGTGCAAAAAAAGCTCCCAATCATCCAAAAAATTATT
ATCATGGATTCTAAAACGGATTACCAGGGATTTCAGTCGATGTACACGTT
CGTCACATCTCATCTACCTCCCGGTTTTAATGAATACGATTTTGTGCCAG
AGTCCTTCGATAGGGACAAGACAATTGCACTGATCATGAACTCCTCTGGA
TCTACTGGTCTGCCTAAAGGTGTCGCTCTGCCTCATAGAACTGCCTGCGT
GAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATCTATAG
ATTTGAAGAAGAGCTGTTTCTGAGGAGCCTTCAGGATTACAAGATTCAAA
GTGCGCTGCTGCTGCCAACCCTATTCTCCTTCTTCGCCAAAAGCACTCTG
ATTCACAAATACGATTTATCTAATTTACACGAAATTGCTTCTGGTGGCGC
TCCCCTCTCTAAGGAAGTCGGGGAAGCGGTTGCCAAGAGGTTCCATCTGC
CAGGTATCAGGCAAGGATATCGGCTCACTGAGACTACATCAGCTATTCTG
ATTACACCCGAGGGGGATGATAAACCGGGCGCGGTCGGTAAACTTGTTCC
ATTTTTTGAAGCGAAGGTTGTGCATCTGGATACCCCCAAAACGCTCCCCC
TTAATCAAACAGGCGAACTCTCTGTGACACGTCCTATGATTATGTCCCCT
TATGTAAACAATCCGGAAGCCACCAACGCCTTGATTGACAAGGATGGATG
GCTACATTCTGGAGACATAGCTTACTGGGACGAAGACGAACACTTCTTCA
TCGTTGACCGCCTGAAGTCTCTGATTAAGTACAAAGGCTATCAGGTGGCT
CCCGCTGAATTGGAATCCATCTTGCTCCAACACCCCAACATCTTCGACGC
AGGTGTCGCAGGTCTTCCCGACGATGACGCCCGTGAACTTCCCGCCGCCG
TTGTTGTTTTGGAGCACGGAAAGACGATGACGGAAAAAGAGATCGTGGAT
TACGTCGCCAGTCAAGTAACAACCGCCAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGATCGCCGTG
TAA,

the SV40-derived polyadenylation signal is the region represented by the following sequence:

(SEQ ID NO: 15)

CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGGTGTGGGAGGTTTTTTAAAGCAAG
TAAAACCTCTACAAATGTGGTA,
and

the Right-ITR is the region represented by the following sequence:

(SEQ ID NO: 16)

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG.

pAAV-RC (STRATAGENE AAV Helper-Free System Cat#240071) coding for the adeno-associated virus-derived rep and cap genes was used as a helper plasmid comprising the genes that are necessary for virus reproduction and viral particle formation (rep and cap).

The base sequence of the plasmid pAAV-RC is shown below.

(SEQ ID NO: 17)

GCGCGCCGATATCGTTAACGCCCCGCGCCGGCCGCTCTAGAACTAGTGGA
TCCCCCGGAAGATCAGAAGTTCCTATTCCGAAGTTCCTATTCTCTAGAAA
GTATAGGAACTTCTGATCTCCGCAGCCGCCATGCCGGGGTTTTACGAGAT
TGTGATTAAGGTCCCCAGCGACCTTGACGAGCATCTGCCCGGCATTTCTG
ACAGCTTTGTGAACTGGGTGGCCGAGAAGGAATGGGAGTTCCCGCCAGAT
TCTCACATGGATCTCAATCTGATTGACCAGCCACCCCTGACCCTCGCCCA
CAACCTGCAGCGCGACTTTCTGACGGAATGGGGGGGTGTGAGTAAGGCCC
CGGAGGCGGTTTTGTTTGTGCAATTTGAGAAGGGAGAGAGCTACTTCCAC
ATGCACGTGCTCGTGGAAACCACCGGGGTGAAATCCATGGTTTTGGGACG
TTTCCTGAGTCAGATTCGCGAAAAACTGATTCAGAGAATTTACCGCGGGA
TCGAGCCGACTTTGCCAAACTGGTTCGCGGTCACAAAGACCAGAAATGGC
GCCGGAGGCGGGAACAAGGTGGTGGATGAGTGCTACATCCCCAATTACTT
GCTCCCCAAAACCCAGCCTGAGCTCCAGTGGGCGTGGACTAATATGGAAC
AGTATTTAACCGCCTGTTTGAATCTCACGGAGCGTAAACGGTTGGTGGCG
CAGCATCTGACGCACGTGTCGCAGACGCAGGAGCAGAACAAAGAGAATCA
GAATCCCAATTCTGATGGGCCGGTGATCAGATCAAAAACTTCAGCCAGGT
ACATGGAGCTGGTCGGGTGGCTCGTGGACAAGGGGATTACCTCGGAGAAG
CAGTGGATCCAGGAGGACCAGGCCTCATACATGTCCTTCAATGCGGCCTC
CAACTCGCGGTCCCAAATCAAGGCTGCCTTGGACAATGCGGGAAAGATTA
TGAGCCTGACTAAAACCGCCCCCGACTACCTGGTGGGCCAGCAGCCCGTG
GAGGACATTTCCAGCAATCGGATTTATAAAATTTTGGAACTAAACGGGTA
CGATCCCCAATATGCGGCTTCCGTCTTTCTGGGATGGCCCACGAAAAAGT
TCGGCAAGAGGAAGACCATCTGGCTGTTTGGGCCTGCAACTACCGGGAAC
ACCAACATCGCGGAGGCCATAGCCCACAGTGTGCCGTTCTACGGGTGCGT
AAAGTGGACCAATGAGAACTTTCCCTTCAACGACTCTGTCGACAAGATGG
TGATCTGGTCGGAGGAGGGGAAGATGACCGCCAAGGTCGTGGAGTCGGCC
AAAGCGATTCTCGGAGGAAGCAAGGTGCGCGTGGAGCAGAAATGCAAGTG
GTCGGCCCAGATAGACCCGAGTCCGGTGATGGTCACGTGGAACAGGAAGA
TGTGGGCGGTGATTGAGGGGAAGTCAAGGAGGTTGGAAGAGGACGAGGGG
TTGGAAGAGGGGATGTTGAAATTTGAAGTGAGGGGGGGTGTGGATGATGA
GTTTGGGAAGGTGAGGAAGGAGGAAGTGAAAGAGTTTTTGGGGTGGGGAA
AGGATGAGGTGGTTGAGGTGGAGGATGAATTGTACGTGAAAAAGGGTGGA
GGGAAGAAAAGAGGGGGGGGGAGTGAGGGAGATATAAGTGAGGGGAAAGG
GGTGGGGGAGTGAGTTGGGGAGGGATGGAGGTGAGAGGGGGAAGGTTGGA
TGAAGTAGGGACAGAGGTAGGAAAAGAAATGTTGTGGTCAGGTGGGGATG
AATGTGATGGTGTTTGGGTGGAGAGAATGCGAGAGAATGAATGAGAATTG
AAATATGTGGTTCACTCACGGAGAGAAAGAGTGTTTAGAGTGGTTTCCCG
TGTGAGAATCTCAAGGGGTTTGTGTGGTGAAAAAGGGGTATCAGAAAGTG
TGGTAGATTGATGATATGATGGGAAAGGTGGGAGAGGGTTGGAGTGGGTG
GGATGTGGTGAATGTGGATTTGGATGAGTGGATGTTTGAACAATAAATGA
TTTAAATGAGGTATGGGTGGGGATGGTTATGTTGGAGATTGGGTGGAGGA
GAGTGTCTCTGAAGGAATAAGAGAGTGGTGGAAGGTGAAAGGTGGCCGAG
GAGGAGGAAAGCCCGCACACCGGCATAACCACGACACCAGGGGTGTTGTC
GTTGCTGGCTACAACTACCTCGCACGCTTCAACGCACTCGACAAGGGAGA
GCCGGTCAACGACGCAGACCCCCCGCCCCTCGACCACGACAAACCCTACG
ACCCCCAGCTCGACAGCGGACACAACCCGTACCTCAAGTACAACCACCCC
GACCCCCAGTTTCACGAGCGCCTTAAAGAAGATACCTCTTTTGCGGGCAA
CCTCGCACGAGCAGTCTTGCAGCCGAAAAAGACGGTTCTTGAACCTCTGG
GCCTGGTTGACGAACCTGTTAACAGGCCTCCGGGAAAAAAGAGCCCGCTA
GAGCACTGTCCTGTGGAGCCAGACTCCTCCTCCCCAACCGGAAAGGCGGG
CCACCACCCTCCAAGAAAAAGATTGAATTTTGCTCAGACTGCAGACCCAG
ACTCAGTACCTGACCCGCAGGCTCTCGGACAGCCACCACCAGCCCGCTCT
GCTCTGGGAACTAATACCATCCCTACAGCCACTCCCCCACCAATCCCAGA
CAATAACGAGGGCCCCGACGGAGTGGGTAATTCCTCGCCAAATTGCCATT
CCGATTCCACATCCATGGGCGACACAGTCATCACCACCACCACCCCAACC
TCCCCCCTCCCCACCTACAACAACCACCTCTACAAACAAATTTCCACCCA
ATCACCACCCTCCAACCACAATCACTACTTTCCCTACACCACCCCTTCCC
CCTATTTTCACTTCAACACATTCCACTCCCACTTTTCACCACCTCACTCC
CAAACACTCATCAACAACAACTCCCCATTCCCACCCAACACACTCAACTT
CAACCTCTTTAACATTCAACTCAAACACCTCACCCACAATCACCCTACCA
CCACCATTCCCAATAACCTTACCACCACCCTTCACCTCTTTACTCACTGC
CACTACCACGTCCCCTACCTCCTGCCGTCCCCCCATCAACCATCGGTCCC
CCCCTTCCCACCACACCTGTTCATCCTCCCACACTATCCATACGTGACCG
TCAACAACCCCACTCACCGACTACCACCGTGTTCATTTTACTCGGTCCAC
TACTTTCCTTGTGACATCGTCGCTACGCCAAACAACTTTACGTTCACCTA
CACTTTTCACCACCTTGGTTTCCACACCACGTACCGTCACACCCACACTC
TCCACCCTCTCATCAATCCTCTCATCCACCACTACCTCTATTACTTCACC
ACAACAAACACTCCAACTCCAACCACCACCCACTCAACCCTTCACTTTTC
TCACCCCCCACGCACTCACATTCCCCACCACTCTACCAACTCCCTTCGTC
CACCGTCTTACCCCCACCACGCACTATCAAACACATCTCCCCATAACAAC
AACACTCAATACTCCTCCACTCCACCTACCAACTACCACGTCAATCCCAC
ACACTCTCTCCTCAATCGCCCCCCCCCCATCCCAACGCACAACCACCATC
AACAAAACTTTTTTCCTCACACCCCCCTTCTCATCTTTCCCAACCAACCC
TCACACAAAACAAATCTCCACATTCAAAACCTCATCATTACACACCAACA
CCAAATCACCACAACCAATCCGCTCCCTACCCACCACTATCCTTGTCTAT
CTACCAACCTCCACACACCCAACACACAACCACCTACCCCACATCTCAAC
ACACAACCCCTTCTTCCACCCATCCTCTCCCACCACACACATCTCTACCT
TGACCCCCGCATCTCCCCAAACATTCCACACACCCACCCACATTTTGACG
CGTGTGCCCTCATGGGTGGATTCGGACTTAAACACCCTCCTCCACACATT
CTCATCAAGAACACCCCCGTACCTGCGAATCCTTCGACCACCTTCAGTCC
CGCAAAGTTTGCTTCCTTCATCACACAGTACTCCACCGCACAGCTCAGCG
TGGAGATCGAGTGGGAGCTGCACAACCAAAACAGCAAACGCTGCAATCCC
GAAATTCAGTACACTTCCAACTACAACAAGTCTCTTAATCTCCACTTTAC
TGTGGACACTAATCCCCTGTATTCAGAGCCTCGCCCCATTGGCACCAGAT
ACCTGACTCCTAATCTCTAATTGCTTGTTAATCAATAAACCGTTTAATTC
GTTTCAGTTCAACTTTGGTCTCTGCGTATTTCTTTCTTATCTAGTTTCCA
TCGCTACGTACATAAGTACCATGCCGGGTTAATCATTAACTACACCCCCC
CCGTTTAAACAGCGCGCGGACGCGTGCACTCCTCACGTGAATTACGTCAT
AGCGTTAGCCAGCTCCTGTATTACAGCTCACCTGAGTCTTTTGCGACATT
TTGCGACACCATCTGGTCTCGCTCCGGGGGCGGCCCCGAGTGACCACCCA
GCCTCTCCATTTTGAAGCGGGAGGTTTCAACCAGCCCTCGCGCGCTCACT
CCCCGTCCTTTTACAACGTCGTGACTGGGAAAACCCTGGCCTTACCCAAC
TTAATCCCCTTCCACCACATCCCCCTTTCCCCAGCTGCCCTAATAGCGAA
GAGGCCCGCACCCATCCCCCTTCCCAACAGTTGCCCACCCTCAATGGCGA
ATGGAAATTGTAAGCGTTAATATTTTGTTAAAATTCGCCTTAAATTTTTC
TTAAATCACCTCATTTTTTTAACCAATAGGCCGAAATCGGCAAAATCCCT
TATAAATCAAAACAATAGACCCAGATAGGGTTGACTGTTGTTCCACTTTG
GAACAAGACTCCACTATTAAGAACCTGGACTCCAACGTCAAACGGCGAAA
AACCGTCTATCAGGGCGATGGCCCACTACGTCAACCATCACCCTAATCAA
CTTTTTTGGGGTCGAGGTGCCGTAAAGCACTAAATCGGAACCCTAAACGC
AGCCCCCGATTTAGAGCTTGACCCCCAAAGCCCGCGAACGTCCCGACAAA
CCAAGGGAAGAAAGCCAAACCACCCCCCGCTAGGGCGCTCCCAACTCTAG
CGGTCACCCTCCGCCTAACCACCACACCCCCCGCCCTTAATCCGCCGCTA
CACCCCCCGTCAGGTGGCACTTTTCCCCCAAATGTGCGCGCAACCCCTAT
TTGTTTATTTTTCTAAATACATTCAAATATCTATCCGCTCATCACACAAT
AACCCTCATAAATCCTTCAATAATATTGAAAAAGGAAGAGTATCACTATT
CAACATTTCCGTCTCCCCCTTATTCCCTTTTTTCCCCCATTTTGCCTTCC
TCTTTTTGCTCACCCAGAAACGCTCCTCAAACTAAAACATCCTCAACATC
ACTTCCCTCCACCACTCCCTTACATCCAACTCCATCTCAACACCCCTAAC
ATCCTTCACACTTTTCCCCCCCAACAACCTTTTCCAATCATCACCACTTT
TAAACTTCTCCTATCTCCCCCCCTATTATCCCCTATTCACCCCCCCCAAC
ACCAACTCCCTCCCCCCATACACTATTCTCACAATCACTTCCTTCACTAC
TCACCACTCACACAAAACCATCTTACCCATCCCATCACACTAACACAATT
ATCCACTCCTCCCATAACCATCACTCATAACACTCCCCCCAACTTACTTC
TCACAACGATCGGAGGACCGAACCAGCTAACCCCTTTTTTCCACAACATG
GGGGATCATGTAACTCCCCTTGATCGTTCCCAACCCCACCTCAATGAACC
CATACCAAACCACCAGCGTGACACCACCATGCCTCTAGCAATCGCAACAA
CGTTGCGCAAACTATTAACTCGCCAACTACTTACTCTAGCTTCCCGGCAA
CAATTAATACACTGCATGGACGCCGATAAACTTGCAGGACCACTTCTCCG
CTCGGGCCTTCCGGGTCGCTGGTTTATTCCTGATAAATCTGGAGCCCCTC
AGCGTGGCTCTCCGCGTATCATTGCACCAGTGGGGCCAGATGCTAACCCC
TCCCGTATCGTAGTTATGTACACGACGGGCAGTGACCCAACTATGGATCA
ACGAAATAGACAGATCGCTGACATAGGTCCCTGAGTGATTAAGCATTGGT
AACTCTCACACCAAGTTTACTCATATATACTTTAGATTGATTTAAAAGTT
CATTTTTAATTTAAAAGCATCTAGGTGAAGATCCTTTTTCATAATCTCAT
GAGCAAAATCCCTTAACGTGACTTTTCGTTCCACTCACCCTCAGACCCCC
TACAAAAGATCAAAGCATGTTGTTCAGATCCTTTTTTTCTGCGCCTAATC
TGCTCCTTCCAAACAAAAAAACCACCCCTACCACGCGTGGTTTGTTTCCC
CGATCAAGACCTACCAACTGTTTTTCCGAAGGTAACTCGCTTCACCACAG
CCCAGATACCAAATACTCTTCTTCTACTGTAGCCCTAGTTAGCCCACCAC
TTCAAGAACTCTCTAGCACCGCCTACATACCTCCCTCTCCTAATGCTGTT
ACCAGTGGCTGCTGCCAGTCCCGATAAGTCGTGTCTTACCGGGTTCCACT
CAACACGATAGTTACCCCATAAGCCGCACCCGTCGCGCTGAACGGGGCGT
TCCTGGACACACCCCAGCTTGGACCCAACCAGCTACACCCAACTCAGATA
CCTACAGCGTGAGCTATGACAAAGCGCCACCCTTCCCGAAGGGACAAACG
CGCACACCTATCCGGTAACCCGCAGGCTCCCAACAGCAGAGCGCACGACG
GAGCTTCCAGGCGGAAACGGCTCGTATCTTTATACTCCTCTCCCCTTTCG
CCACCTCTGACTTCACCGTCGATTTTTGTGATCGTGCTCACCCCCCCCCA
CCCTATCCAAAAACCCCACCAACCCCCCCTTTTTACCCTTCCTCCGCTTT
TCGTCCCCTTTTCGTCACATCTTCTTTCCTCGCTTATCGCGTCATTGTCT
CCATAACGCTATTACCCGGTTTCACTCACCTCATACCCGTCCCCCGACCC
CAACCACGCACGCGACGCACTGACTCACGCACCAACCCCAACACCCCCGA
ATACCCAAAGCCGCTGTGCCGCGCGCTTCCCCCATTGATTAATCCACCTC
CCACCACACCTTTCCCCACTCCAAACGCCCCACTCACGCGAAGCGAATTA
ATCTCACTTACGTCACTCATTACCGACCCCACCGTTTACAGTTTATCCTT
CCCCCTCCTATCTTCTCTCCAATTCTCACGCCATAACAATTTCACACACC
AAACACGTATCACGATCATTACCCCAA

pHelper (STRATAGENE AAV Helper-Free System Cat#240071) coding for the adenovirus-derived VA, E2A and E4 genes was used as an adenovirus gene expression plasmid comprising the adenoviral genes that are necessary for the production of adeno-associated virus vector (E2A, E4 and VA).

The base sequence of the plasmid pHelper is shown below.

(SEQ ID NO: 18)

GGTACCCAACTCCATGCTTAACAGTCCCCACGTACAGCCCACCCTGCGTC
CCAACCAGGAACAGCTCTACAGCTTCCTGGAGCGCCACTCCCCCTACTTC
CGCAGCCACAGTGCGCAGATTAGGAGCGCCACTTCTTTTTCTCACTTGAA
AAACATCTAAAAATAATGTACTAGGAGACACTTTCAATAAAGGCAAATGT
TTTTATTTGTACACTCTCCGCTGATTATTTACCCCCCACCCTTGCCGTCT
GCGCCGTTTAAAAATCAAAGGGCTTCTCCCGCGCATCGCTATCCGCCACT
GGCAGGGACACGTTGCCATACTGGTGTTTACTGCTCCACTTAAACTCAGG
CACAACCATCCGCGGCAGCTCGGTCAACTTTTCACTCCACACGCTGCCCA
CCATCACCAACCCCTTTAGCAGCTCGGGCCCCCATATCTTGAACTCCCAC
TTGGGGCCTCCCCCCTGCGCGCGCGAGTTGCCATACACAGGGTTCCAGCA
CTGGAACACTATCACCGCCGGCTCGTGCACGCTCGCCACCACGCTCTTGT
CGGAGATCAGATCCGCGTCCAGGTCCTCCGCGTTGCTCAGGCCGAACGGA
GTCAACTTTGGTAGCTGCCTTCCCAAAAAGGGTGCATGCCCAGGCTTTGA
CTTCCACTCGCACCGTACTGGCATCAGAAGGTCACCGTGCCCGGTCTGGG
CGTTAGGATACAGCCCCTGCATCAAAGCCTTGATCTGCTTAAAACCCACC
TCACCCTTTCCCCCTTCACACAACAACATCCCCCAACACTTCCCCCAAAA
CTCATTGGCCCGACACGCCCCCTCATCCACGCAGCACCTTCCCTCGCTCT
TCCACATCTCCACCACATTTCGCCCCCACCCGTTCTTCACCATCTTGGCC
TTCCTACACTCCTCCTTCAGCGCGCGCTGCCCCTTTTCCCTCCTCACATC
CATTTCAATCACGTGCTCCTTATTTATCATAATGCTCCCGTCTACACACT
TAAGCTCCCCTTCGATCTCACCGCACCGGTGCAGCCACAACGCGCAGCCC
CTCGGCTCGTCCTCCTTGTACCTTACCTCTCCAAACCACTGCACGTACGC
CTCCAGGAATCCCCCCATCATCGTCACAAAGGTCTTCTTGCTCGTCAACC
TCACCTGCAACCCCCGCTGCTCCTCGTTTAGCCAGCTCTTGCATACCCCC
CCCACAGCTTCCACTTCGTCAGGCAGTACCTTGAACTTTCCCTTTAGATC
GTTATCCACGTGGTACTTCTCCATCAACCCCCCCGCACCCTCCATCCCCT
TCTCCCACCCACACACCATCCCCAGCCTCAGCGGGTTTATCACCGTCCTT
TCACTTTCCCCTTCACTCGACTCTTCCTTTTCCTCTTGCGTCCCCATACC
CCCCGCCACTCCCTCCTCTTCATTCACCCCCCGCACCCTGCGCTTACCTC
CCTTCCCGTGCTTGATTACCACCGGTCCCTTGCTGAAACCCACCATTTGT
AGCGCCACATCTTCTCTTTCTTCCTCGCTGTCCACCATCACCTCTCCGCA
TCCCCCGCGCTCCGGCTTGGCAGAGCGGCCCTTCTTTTTCTTTTTGGACG
CAATGGCCAAATCCCCCCTCGAGGTCCATGGCCGCCGGCTCGCTGTGCCC
CGCACCACCGCATCTTCTGACCAGTCTTCTTCCTCCTCGGACTCGAGACC
CCGCCTCAGCCGCTTTTTTGGGGGCCCGCGGCCAGGCCGCCGCGACGCCG
ACGGGCACGACACCTCCTCCATGGTTGGTGGACGTCGCCCCCCACCCCGT
CCGCCCTCGGGCGTGGTTTCGCGCTGCTCCTCTTCCCGACTGCCCATTTC
CTTCTCCTATAGCCAGAAAAACATCATCGAGTCAGTCGAGAAGGAGGACA
CCCTAACCGCCCCCTTTGACTTCGCCACCACCCCCTCCACCGATGCCCCC
AACCCCCCTACCACCTTCCCCGTCCACGCACCCCCGCTTGACGAGCAGGA
AGTGATTATCCACCACCACCCACCTTTTCTAACCCAACACCACCACCATC
CCTCACTACCAACACACCATAAAAACCAACACCACCACCACCCACACCCA
AACCACCAACAACTCCCCCCCCCCCACCAAACCCATCCCCACTACCTACA
TCTCCCACACCACCTCCTCTTCAACCATCTCCACCCCCACTCCCCCATTA
TCTCCCACCCCTTCCAACACCCCACCCATCTCCCCCTCCCCATACCCCAT
CTCACCCTTCCCTACCAACCCCACCTCTTCTCACCCCCCCTACCCCCCAA
ACCCCAACAAAACCCCACATCCCACCCCAACCCCCCCCTCAACTTCTACC
CCCTATTTCCCCTCCCACACCTCCTTCCCACCTATCACATCTTTTTCCAA
AACTCCAACATACCCCTATCCTCCCCTCCCAACCCCACCCCACCCCACAA
CCACCTCCCCTTCCCCCACCCCCCTCTCATACCTCATATCCCCTCCCTCC
ACCAACTCCCAAAAATCTTTCACCCTCTTCCACCCCACCACAAACCCCCC
CCAAACCCTCTCCAACAACAAAACACCCAAAATCAAACTCACTCTCCACT
CCTCCTCCAACTTCACCCTCACAACCCCCCCCTACCCCTCCTCAAACCCA
CCATCCACCTCACCCACTTTCCCTACCCCCCACTTAACCTACCCCCCAAC
CTTATCACCACACTCATCACCCACCTCATCCTCCCCCCTCCACCACCCCT
CCACACCCATCCAAACTTCCAACAACAAACCCACCACCCCCTACCCCCAC
TTCCCCATCACCACCTCCCCCCCTCCCTTCACACCCCCCACCCTCCCCAC
TTCCACCACCCACCCAACCTAATCATCCCCCCACTCCTTCTTACCCTCCA
CCTTCACTCCATCCACCCCTTCTTTCCTCACCCCCACATCCACCCCAACC
TACACCAAACCTTCCACTACACCTTTCCCCACCCCTACCTCCCCCACCCC
TCCAAAATTTCCAACCTCCACCTCTCCAACCTCCTCTCCTACCTTCCAAT
TTTCCACCAAAACCCCCTCCCCCAAAACCTCCTTCATTCCACCCTCAACC
CCCACCCCCCCCCCCACTACCTCCCCCACTCCCTTTACTTATTTCTCTCC
TACACCTCCCAAACCCCCATCCCCCTCTCCCACCAATCCCTCCACCACCC
CAACCTAAACCACCTCCACAACCTCCTAAACCAAAACTTCAACCACCTAT
CCACCCCCTTCAACCACCCCTCCCTCCCCCCCCACCTCCCCCACATTATC
TTCCCCCAACCCCTCCTTAAAACCCTCCAACACCCTCTCCCACACTTCAC
CAGTCAAAGCATGTTGCAAAACTTTAGGAACTTTATCCTAGAGCGTTCAG
GAATTCTGCCCCCCACCTGCTGTCCGCTTCCTAGCCACTTTCTCCCCATT
AACTACCCTCAATGCCCTCCGCCGCTTTGGGGTCACTGCTACCTTCTGCA
GCTAGCCAACTACCTTCCCTACCACTCCCACATCATGGAAGACGTCACCC
CTGACCCCCTACTGGACTCTCACTGTCGCTGCAACCTATGCACCCCGCAC
GGGTCCCTGGTCTGCAATTCGCAACTGCTTAGCGAAAGTCAAATTATCGG
TACCTTTCAGCTCCAGGCTCCCTCGCCTCACCAAAAGTCCCCGCCTCCGC
CGTTGAAACTCACTCCGGGGCTGTCCACGTCGCCTTACCTTCGCAAATTT
CTACCTCACCACTACCACGCCCACGAGATTAGCTTCTACGAAGACCAATC
CCGCCCGCCAAATGCGCAGCTTACCGCCTCCGTCATTACCCAGCGCCACA
TCCTTCGCCAATTGCAACCCATCAACAAAGCCCCCCAAGAGTTTCTGCTA
CGAAACCGACCCGGGGTTTACCTGGACCCCCAGTCCGCCGACGAGCTCAA
CCCAATCCCCCCCCCGCCGCACCCCTATCAGCAGCCCCCCGCCCTTGCTT
CCCAGGATGCCACCCAAAAACAAGCTCCACCTCCCGCCCCCCCCACCCAC
GGACGAGGAGGAATACTCCGACACTCAGCCAGACGAGGTTTTGCACCAGC
AGGAGGAGATGATCCAACACTGCCACACCCTAGACGAAGCTTCCGAGCCC
GAACAGGTGTCAGACCAAACACCCTCACCCTCGCTCGCATTCCCCTCGCC
GGCGCCCCACAAATTGGCAACCGTTCCCACCATCCCTACAACCTCCGCTC
CTCAGCCGCCCCCGGCACTGCCTGTTCGCCGACCCAACCCTAGATCGCAC
ACCACTGGAACCACGGCCGGTAAGTCTAAGCACCCGCCGCCGTTAGCCCA
AGAGCAACAACACCGCCAAGGCTACCGCTCGTGGCGCCGGCACAACAACG
CCATAGTTGCTTGCTTGCAAGACTGTCCGGCCAACATCTCCTTCCCCCGC
CCCTTTCTTCTCTACCATCACGGCCTCGCCTTCCCCCGTAACATCCTGCA
TTACTACCGTCATCTCTACACCCCCTACTCCACCCCCGCCACCGGCAGCC
CCACCAACACCAGCGGTCACACAGAAGCAAAGGCGACCGGATAGCAAGAC
TCTGACAAAGCCCAAGAAATCCACACCGGCCCCACCACCAGCAGGACCAC
CGCTCCGTCTGGCCCCCAACCAACCCCTATCGACCCCCGACCTTAGAAAT
AGCATTTTTCCCACTCTCTATGCTATATTTCAACAAAGCAGGCCCCAACA
ACAAGACCTGAAAATAAAAAACAGCTCTCTCCCCTCCCTCACCCCCACCT
CCCTCTATCACAAAACCCAACATCACCTTCCCCCCACCCTCCAACACCCC
CACCCTCTCTTCACCAAATACTCCCCCCTCACTCTTAACCACTACTTTCC
CCCCCTTTCTCAAATTTAACCCCCAAAACTACCTCATCTCCACCCCCCAC
ACCCCCCCCCACCACCTCTCCTCACCCCCATTATCACCAACCAAATTCCC
ACCCCCTACATCTCCACTTACCACCCACAAATCCCACTTCCCCCTCCACC
TCCCCAACACTACTCAACCCCAATAAACTACATCACCCCCCCACCCCACA
TCATATCCCCCCTCAACCCAATCCCCCCCCACCCAAACCCAATTCTCCTC
GAAGAGGCGGCTATTAGCACCACAGCTCGTAATAACCTTAATCCCCGTAG
TTGGCCCGCTGGCCTGGTGTACCAGGAAAGTCCCGCTCCCACCACTCTCG
TACTTCGCAGACACGCCCAGCGGGAAGTTCAGATGAGTAAGTCAGGGGCC
CACCTTGGGGGGGGGTTTCGTCACAGGGTGCCGTCCCCGGGGCCTTTTAG
GGCGGAGTAACTTGCATCTATTGCCAATTGTAGTTTTTTTAAAATGGGAA
GTGACGTATCGTGGGAAAACGGAAGTGAAGATTTGAGGAAGTTGTGGGTT
TTTTGGCTTTCGTTTCTGGGCGTAGGTTCGCGTGCGGTTTTCTGGGTGTT
TTTTGTGGACTTTAACCGTTACGTGATTTTTTAGTGGTATATATAGTCGC
TCTGTAGTTGGCCCTTTTTACACTGTGACTGATTGAGCTGGTGCCGTGTC
GAGTGGTGTTTTTTAATAGGTTTTTTTACTGGTAAGGCTGACTGTTATCG
GTCGCGGTGTCGAAGGCCTGTATGTTGTTGTGCACGGGGAGGCTGCTATT
TTGGCTAGGCAGCAGCGTTTTTCACGTGTTTATGTGTTTTTGTGTCCTAT
TAATTTTGTTATAGCTCGTATCGCCGCTCTAATCTTCTCTGTACCCCTCC
CCCTATCTATTCCCCCCCCCTATTTGCCTGCCTTTTTACCACTCAGCGAT
CTTAACCAACCTCATCTCTTTACGCACTGTTACATTATGAGTGCGGACAT
CACCCACCAACTGTGGCTCGTCCTTTTTAATCACCCTCACCACTTTTTTT
ACCGTCACCCCGCGATGCGCGTACTCGCTCTTATCCTTATAACCGTTCTT
TTTCCTCTTGTAACAGACCCTTCTAATCTTTAAATGTTTTTTTTTTTCTT
ATTTTATTTTCTCTTTAATGCACCAACCGCGACACATCTTTCAGACAAAA
ATCGTGTCTTTTTCTCTCGTGGTTCCCCAACTTACGTCCCTTTATCTGCA
TCACGATCACTACCATGTGCTTCCTTTTTTGCGCCACCCTTTCCCTCATT
TTTTCACCACCACCTTCCATTTTATATGCCCCGGCATCGAACAACGTTAC
ATACCCCGTACCCTGGTTACCATACGTCCCACTATGCGTGTGATAATCAC
TCTCCCTTCTTTTCTCATCCTTCCTCCCCCCCAACTCCCCCCGCTCGTCG
GTGCACACGTGCACCATTATCTTGACGTGCCGGTGCGAACCCACCTAGCG
CATCCCCCTATTTTTCTTAATCTTCCCCTTTTCAATCTTATAGACCTCTC
TCACCAACCTCAATTTTTCCAATGATCATTCGGTCGTTGACGCTCAACCT
GCACCCCCCTCTGGACGACATTTTTACAATCCCCCGACTTAATATTCCCC
ATTTCGTTACACAGATATTCATAACCTGCCCAGATCAAAATTATTTCCGC
ATCCTTCAACCTGCTCCAATCTTTATACACCAGATTGACCCTCAACCCTT
TACCCTTTAGCTCGACTTGCACCTCACCCCAGTTTCCCTTTTCCAACGGA
TTCTCCAAGATCTTACAAATCCCATTATGTGTTCTTTCGGTCTACACTTT
CACCACGCCACGGGACGCCACCGCGTTGAGTTAATAGATGTTGATTTTCA
GGTTTTCCATAATGTTTTCGAATAAAAAAAAAAAAACATCGTTCTTCCAG
GTGTTCCCCCTCCTCCCGTGTCTGACTCCCACAACGAATGTGTACGTTCG
GTCGGTCTCCCTTATTCTCCCGTGCTCGATGTTATCACCGCACGGCCGCA
TGAAGGACTTTACATAGAACCCGAAGCCAGGGGGCGCCTGGATCCTTTCA
CAGAGTCGATATACTACAACTACTACACACAGCCACCTAAGCGACGAGAC
CCGACACCCAGATCTCTTTCTCACCCCCGCACCTCGTTTTCCTTCACGAA
ATATGACTACGTCCGGCGTTCCATTTCCCATGACACTACCACCAACACCA
TCTCGGTTGTCTCGGCGCACTCCGTACACTACCGATCGCCTACCTCCTTT
TGAGACACAGACCCGCGCTACCATACTCCAGGATCATCCCCTGCTCCCCG
AATCTAACACTTTGACAATCCACAACGTGAGTTACGTGCGAGGTCTTCCC
TCCACTGTCGGATTTACGCTGATTCACCAATCCCTTGTTCCCTGGCATAT
CGTTCTGACGCGGGAGCAGCTTCTAATCCTGAGGAAGTGTATCCACGTGT
CCCTGTCTTGTGCCAACATTGATATCATCACGAGCATCATGATCCATGCT
TACGAGTCCTGCGCTCTCCACTCTCATTCTTCCAGTCCCGCTTCCCTGCA
CTCCATAGCCGGCGGGCACCTTTTGGCCAGCTGGTTTAGGATGGTGGTGG
ATGGCGCCATGTTTAATCACACCTTTATATCCTACCCCCACCTCCTCAAT
TACAACATCCCAAAACACCTAATCTTTATCTCCACCCTCTTTATCACCCC
TCCCCACTTAATCTACCTCCCCTTCTCCTATCATCCCCACCTCCCTTCTC
TCCTCCCCCCCATCACCTTTCCATACACCCCCTTCCACTCTCCCATTTTC
AACAATATTCTCCTCCTCTCCTCCACTTACTCTCCTCATTTAACTCACAT
CACCCTCCCCTCCTCTCCCCCCACCACAACCCCTCTCATCCTCCCCCCCC
TCCCAATCATCCCTCACCACACCACTCCCATCTTCTATTCCTCCACCACC
CACCCCCCCCCCCACCACTTTATTCCCCCCCTCCTCCACCACCACCCCCC
TATCCTCATCCACCATTATCACTCTACCCCCATCTACCCCTCCACTTCCC
CTTCCCCCCCCCTTCACCAACCCCAACTTCCACACCACCCTCTCCCTCAC
CACCTCCACACCCACATCAACTTAACCCACCTCCCCCCCCACTTTATTAA
TATCACTCATCACCCTTTCCCTCCACACCAAACCCTCTCCAATATAACAC
CTAACAATATCTCTCTTACCCATCATATCATCCTTTTTAACCCCACCCCC
CCACAAACCACTCTCTACTCTCTCTCTTCCCACCCACCTCCCACCTTCAA
TACTACCCTTCTCTCACTTTCATTAACCTACCCTCATCAATATAACCTAT
CTCCTCCTCCCCCTATACTACTCAATCAAAAATCACTTCAAATTTTCTCC
AATTCAAAAATAAACACCTTCAAACATAACATCCAACACCTTCACCATTC
TTTATTCCTCCCCAATCTACCACAACCTCTAACACTTCCTACCAAAACTT
TCACTCCTCTATTTTCCACTTTCCCACCACCATCTAAAACACATACACTA
ACTCCTTACCTCCCTACTTTCTCTCCATTCACTACAATCCATCTACCATC
TTCCCCCTCCTCACCCCCTACCACAACCCCACCCTCCCCTCCATCTCTCC
CCCTCCTCTTCCTCTTCCCCCTCCTCACCACCCCCCCCCATCTCCCCCAC
CACCCCATCCATCTCCCAAAACCAAAAAACCCCCTCCTCCCTCTTTCCCC
ACCAATTTCCAACCCCCCTCTTCCATCACCCCCACCCAAACCCCCGTTCG
CCGCAGTCCGCCCGGCCCGAGACTCGAACCCGGGGTCCTCCCACTCAACC
CTTGCAAAATAACCCTCCCGCTACAGGCAGCGACCCACTTAATCCTTTCC
CTTTCCAGCCTAACCGCTTACGCCGCGCGCGCCCAGTCCCCAAAAAAGCT
AGCCCAGCAGCCGCCCCCCCTCGAAGCAAGCCAAAACGACCGCTCCCCCG
TTGTCTCACCTCCCACACCTCCCTTCGACACGCGGGCCGTAACCGCATGG
ATCACGGCCCACCGCCCCATCCGGGCTTCGAACCCCGCTCGTCCCCCATG
ATACCCTTCCGAATTTATCCACCAGACCACGGAAGAGTCCCCCCTTACAC
GCTCTCCTTTTCCACGGTCTACACCCTCAACGACTCCCCACGCCTCACCC
GCCAGACCCTCCCGACCATGGAGCACTTTTTCCCCCTCCGCAACATCTCG
AACCCCGTCCCCCACTTTCCCCGCCCCTCCACCACCCCCGCCCGCATCAC
CTGGATGTCCAGCTACATCTACGGATTACGTCGACCTTTAAACCATATGA
TCAGCTCACTCAAAGGCGGTAATACCGTTATCCACAGAATCACGGCATAA
CCCAGGAAAGAACATCTCAGCAAAAGGCCACCAAAACCCCAGGAACCGTA
AAAACCCCGCCTTGCTCGCCTTTTTCCATAGCCTCCCCCCCCCTGACCAC
CATCACAAAAATCGACGCTCAACTCACAGGTGGCGAAACCCGACAGGACT
ATAAACATACCACCCGTTTCCCCCTGGAAGCTCCCTCGTGCGCTCTCCTG
TTCCGACCCTCCCGCTTACCCCATACCTGTCCGCCTTTCTCCCTTCGGGA
ACCCTGGCCCTTTCTCATACCTCACCCTCTACCTATCTCACTTCCCTCTA
CCTCCTTCCCTCCAACCTCCCCTCTCTCCACCAACCCCCCCTTCACCCCC
ACCCCTCCCCCTTATCCCCTAACTATCCTCTTCACTCCAACCCCCTAACA
CACCACTTATCCCCACTCCCACCACCCACTCCTAACACCATTACCACACC
CACCTATCTACCCCCTCCTACACACTTCTTCAACTCCTCCCCTAACTACC
CCTACACTACAACAACACTATTTCCTATCTCCCCTCTCCTCAACCCACTT
ACCTTCCCAAAAACACTTCCTACCTCTTCATCCCCCAAACAAACCACCCC
TCCTACCCGTCCTTTTTTTCTTTCCAACCACCACATTACCCCCACAAAAA
AACCATCTCAACAACATCCTTTCATCTTTTCTACCCCCTCTCACCCTCAC
TCCAACCAAAACTCACCTTAAGCCATTTTCCTCATCAGATTATCAAAAAC
CATCTTCACCTACATCCTTTTAAATTAAAAATGAACTTTTAAATCAATCT
AAACTATATATCACTAAACTTGCTCTCACACTTACCAATCCTTAATCACT
GAGCCACCTATCTCACCCATCTCTCTATTTCCTTCATCCATACTTCCCTC
ACTCCCCCTCCTCTACATAACTACCATACCCCACCCCTTACCATCTCCCC
CCACTCCTCCAATCATACCCCCACACCCACCCTCACCCCCTCCACATTTA
TCACCAATAAACCACCCACCCCCAACCCCCCACCCCACAACTCCTCCTCC
AACTTTATCCCCCTCCATCCACTCTATTAATTCTTCCCCCCAACCTACAC
TAACTACTTCCCCACTTAATACTTTCCCCAACCTTCTTCCCATTCCTACA
CCCATCCTCCTCTCACCCTCCTCCTTTCCTATCCCTTCATTCACCTCCCC
TTCCCAACGATCAACGCGAGTTACATCATCCCCCATCTTCTCCAAAAAAG
CGGTTAGCTCCTTCGGTCCTCCGATCGTTCTCAGAAGTAAGTTGGCCGCA
GTGTTATCACTCATGGTTATGGCAGCACTGCATAATTCTCTTACTGTCAT
CCCATCCGTAACATCCTTTTCTGTGACTCGTCAGTACTCAACCAACTCAT
TCTCACAATACTCTATGCGGCGACCGACTTCCTGTTCCCCGGCGTCAATA
CGGGATAATACCCCCCCACATAGCAGAACTTTAAAAGTCCTCATCATTGG
AAAACGTTCTTCCCCGCCAAAACTCTCAAGGATCTTACCGCTCTTGAGAT
CCAGTTCCATCTAACCCACTCGTGCACCCAACTGATCTTCAGCATCTTTT
ACTTTCACCAGCCTTTCTGGGTGAGCAAAAACAGGAAGGCAAAATGCCGC
AAAAAAGGGAATAAGGCCCACACGCAAATGTTGAATACTCATACTCTTCC
TTTTTCAATATTATTCAAGCATTTATCAGCGTTATTGTCTCATGAGCCCA
TACATATTTGAATGTATTTAGAAAAATAAACAAATAGGCGTTCCCCCCAC
ATTTCCCCGAAAACTGCCACCTAAATTCTAAGCCTTAATATTTTCTTAAA
ATTCGCCTTAAATTTTTGTTAAATCAGCTCATTTTTTAACCAATACCCCC
AAATCCGCAAAATCCCTTATAAATCAAAACAATAGACCCACATAGCCTTG
AGTGTTCTTCCACTTTCGAACAAGACTCCACTATTAAAGAACCTCCACTC
CAACCTCAAACCCCGAAAAACCCTCTATCACGGCCATCCCCCACTACCTC
AACCATCACCCTAATCAAGTTTTTTCCCCTCGAGGTCCCCTAAACCACTA
AATCCGAACCCTAAACGCAGCCCCCCATTTAGACCTTCACGGGCAAAGCC
CCCCAACCTCCCCACAAACCAACGCAACAAACCCAAACCACCGGGCCCTA
CCCCCCTCCCAACTCTACCCCTCACGCTCCGCCTAACCACCACACCCCCC
CCCCTTAATCCGCCGCTACAGGGCCCCATCCATCC

The constructed or acquired three species of plasmids, pAAV-PLS, pAAV-RC and pHelper, were co-transfected by the calcium phosphate method using ProFection Mammalian Transfection System (Promega) into 7×10⁶ 293 cells (derived from HEK293 human embryonic kidney cells stably expressing the adenovirus E1 gene) (STRATAGENE AAV Helper-Free System Cat#240071).

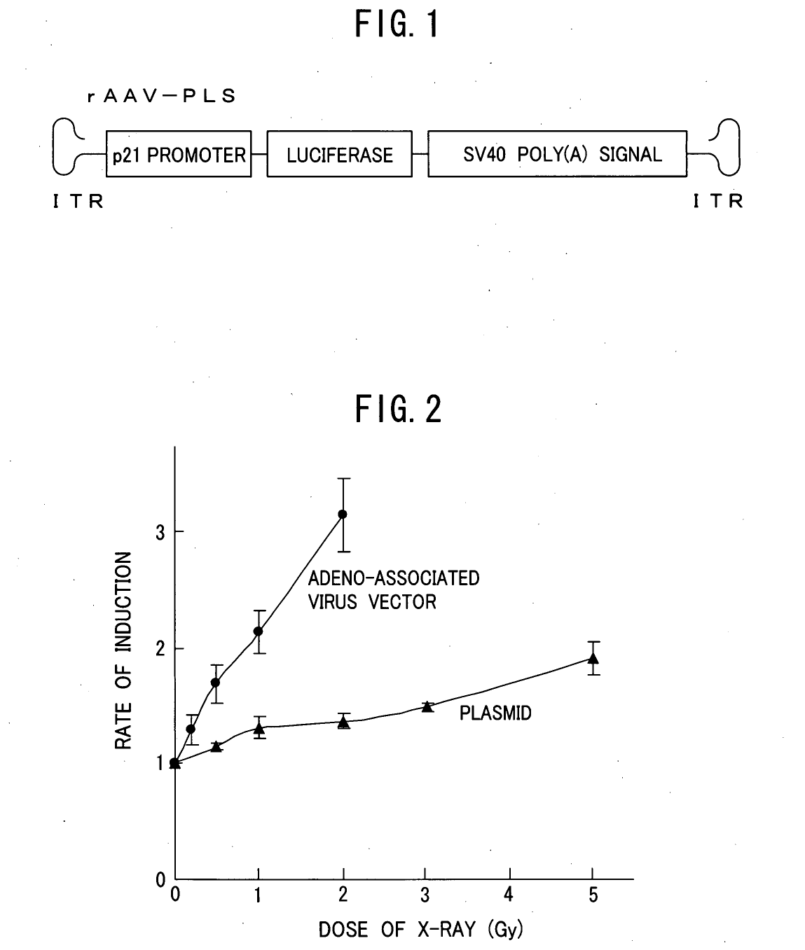

After incubation for three days (culture in 1 ml DMEM containing 10% fetal bovine serum, in an atmosphere at 37° C. containing 5% carbon dioxide), low-dose radiation-inducible viral vector rAAV-PLS produced in 293 cells was recovered by four freeze-thaw cycles (freezing for 10 minutes in ethanol cooled with dry ice, then, melting in a water bath at 37° C.), then centrifuged at 10,000 g for 10 minutes and concentrated. The genome structure of the obtained rAAV-PLS is shown in FIG. 1. The base sequence of rAAV-PLS is shown below.

(SEQ ID NO: 19)

CCTGCAGGCAGCTGCGCGCTCGCTCGCTCACTGAGGCCCCCCGGGCAAAG
CCCGGGCGTCGGGCGACCTTTGGTCGCCCGGCCTCAGTGAGCGAGCCAGC
GCGCAGAGAGGGAGTGGCCAACTCCATCACTAGGGGTTCCTGCGGCCTCG
AGATCTGCGATCTAAGTAAGCTTCCCAGGAACATGCTTGGGCAGCAGGCT
GTGCCTCTGATTGGCTTTCTGGCCGTCAGGAACATGTCCCAACATGTTGA
GCTCTGGCATAGAAGAGGCTGGTGGCTATTTTGTCCTTGGGCTGCCTGTT
TTCAGGTGAGGAAGGGGATGGTAGGAGACAGGAGACCTCTAAAGACCCCA
GGTAAACCTTAGCCTGTTACTCTGAACAGGGTATGTGATCTGCCAGCAGA
TCCTTGCGACAGGGCTGGGATCTGATGCATGTGTGCTTGTGTGAGTGTGT
GCTGGGAGTCAGATTCTGTGTGTGACTTTTAACAGCCTGCTCCCTTGCCT
TTTTCAGGGCAGAAGTCCTCCCTTAGAGTGTGTCTGGGTACACATTCAAG
TGCATGGTTGCAAACTTTTTTTTTTAAAGCACTGAATACTACTACACACT
TAGTAGGTACTTAAGAAATATTGAATGTCGTGGTGGTGGTGAGCTAGAAG
TTATAAAAAAAATTCTTTCCCAAAAACAACAACAAAAAGAATTATTTCAT
TGTGAAGCTCAGTACCACAAAAATTTAAATAATTCATTACAAGCCTTTAT
TAAAAAAAATTTTCTCCCCAAAGTAAACAGACAGACAATGTCTAGTCTAT
TTGAAATGCCTGAAAGCAGAGGGGCTTCAAGGCAGTGGGAGAAGGTGCCT
GTCCTCTGCTGGACATTTGACAACCAGCCCTTTGGATGGTTTGCATGTAT
ACGAGCGAAGGTGCAGACAGCAGTGGGGCTTAGAGTGGGGTCCTGAGGCT
GTGCCGTGGCCTTTCTGGGGTTTAGCCACAATCCTGGCCTGACTCCAGGG
CGAGGCAGGCCAAGGGGGTCTGCTACTGTGTCCTCCCACCCCTACCTGGG
CTCCCATCCCCACAGCAGAGGAGAAAGAAGCCTGTCCTCCCCGAGGTCAG
CTGCGTTAGAGGAAGAAGACTGGGCATGTCTGGGCAGAGATTTCCAGACT
CTGAGCAGCCTGAGATGTCAGTAATTCTAGCTGCTCCAAGCCTGGGTTCT
GTTTTTTAGTGGGATTTCTGTTCAGATGAACAATCCATCCTCTGCAATTT
TTTAAAAGCAAAACTGCAAATGTTTCAGGCACAGAAAGGAGGCAAAGGTG
AAGTCCAGGGGACCTCACGGGTCTGACCTAGATCCGACCGGATAGACACA
TCACTCATTTCTGTCTCTCTCAGAAGAACCAGTACACACTTCCACAATTG
TCCTTTATTTATGTCATCTCCATAAACCATCTGCAAATCAGGGTTATTTC
GCATTTTTCTCATTTTGGACCCACAGAAATAAAGGATGACAAGCACAGAC
CCCCGGGCAGGACGCAAAAGTCCTGTCTTCCAACTATAGTCATTTCTTTC
CTGCATCATCTGACTTACCTCACCACACTTCTCTGAGCCCCACTTTCCCC
AGCAGTGTATACGGGCTATGTGGGGACTATTCAGGAGACAGACAACTCAC
TCCTCAAATCCTCCCCTTCCTGGCCAACAAACCTGCTGCAACCACAGGGA
TTTCTTCTCTTCACCTGACTGTACGCTGTACCCAGATTCGTTCAATGTCC
AATTCTTCTCTTTCCCTCGACATCACCTTGCCCTTTTTTGGTAGTCTCTC
CAATTCCGTCCTTCCCGCAACCATCTGACAATCAACAACTTTGTATACTT
AACTTCAGTCCACCTCAATTTCCTCATCTCTGAAATAAACGCGACTGAAA
AATCATTCTGGCCTCAAGATCCTTTGTTCCGCTGTCTACGTGCTCCAGGT
CCTTCTCCCAGACCTGACCTAGTGAGGGATCAGTCGGAATACAGCTGATA
TTCTGCGGCTTTTCTGGAAATTGCAGACAGGTGCATCGTTTTTATAATTT
ATCAATTTTTATGTATTAATCTCATCCTCCTGATCTTTTCACCTGCATTG
CCTAAATCCTTGCCTCCCAGAGTGGCTCACCCCTCAGCCAGAAAGGGGGC
TCATTCTAACAGTCCTCTGTCCTCCTCGAGAGTGCCAACTCATTCTCCAA
CTAAAAAAAGCCACATTTGTGGCTCACTTCGTGGGCAAATGTGTCCAGCG
CACCAACCCACGCGAGGGACTCGCCGAGGACGGAAGTGCCCTCCTGCAGC
ACGCGAGGTTCCCCGACCCGCTGGCCTGCTCGAACTCCCCCACGCTCAGC
TGGCTCCCCCCTCCCCACCCACCACCCTCCCCCCCCCCCACCCCCCTCCC
CCCCCCCCCCCTCCCCCCACCCCCCCTCCCCCCTCCTTCACCCCCCCCCC
CCCCCCCCCCTTCTATATCACCCCCCCCCTCACCTCCCCCACCTCACCTC
TCACCACCTCCCCAACTCACTTCCTTCTCCAACCTTCCCATTCCCCTACT
CTTCCTAAACCCACCATCCAACACCCCAAAAACATAAACAAACCCCCCCC
CCCATTCTATCCCCTCCAACATCCAACCCCTCCACACCAACTCCATAACC
CTATCAACACATACCCCCTCCTTCCTCCAACAATTCCTTTTACACATCCA
CATATCCACCTCCACATCACTTACCCTCACTACTTCCAAATCTCCCTTCC
CTTCCCACAACCTATCAAACCATATCCCCTCAATACAAATCACACAATCC
TCCTATCCACTCAAAACTCTCTTCAATTCTTTATCCCCCTCTTCCCCCCC
TTATTTATCCCACTTCCACTTCCCCCCCCCAACCACATTTATAATCAACC
TCAATTCCTCAACACTATCCCCATTTCCCACCCTACCCTCCTCTTCCTTT
CCAAAAACCCCTTCCAAAAAATTTTCAACCTCCAAAAAAACCTCCCAATC
ATCCAAAAAATTATTATCATCCATTCTAAAACCCATTACCACCCATTTCA
CTCCATCTACACCTTCCTCACATCTCATCTACCTCCCCCTTTTAATCAAT
ACCATTTTCTCCCACACTCCTTCCATACCCACAACACAATTCCACTCATC
ATCAACTCCTCTCCATCTACTCCTCTCCCTAAACCTCTCCCTCTCCCTCA
TACAACTCCCTCCCTCACATTCTCCCATCCCACACATCCTATTTTTCCCA
ATCAAATCATTCCCCATACTCCCATTTTAACTCTTCTTCCATTCCATCAC
GGTTTTGGAATCTTTACTACACTCGGATATTTGATATCTGGATTTCCAGT
CGTCTTAATGTATAGATTTGAAGAAGAGCTGTTTCTGAGGAGCCTTCAGG
ATTACAAGATTCAAAGTGCGCTGCTGGTGCCAACCCTATTCTCCTTCTTC
GCCAAAAGCACTCTGATTGACAAATACGATTTATCTAATTTACACGAAAT
TGCTTCTGGTGGCGCTCCCCTCTCTAAGGAAGTCGGGGAAGCGGTTGCCA
AGAGGTTCCATCTGCCACGTATCAGGCAAGGATATGGGCTCACTGAGACT
ACATCAGCTATTCTGATTACACCCGAGGGGGATGATAAACCCGGCGCGGT
CGGTAAAGTTGTTCCATTTTTTCAAGCGAAGGTTGTGGATCTGGATACCG
GGAAAACGCTGGGCGTTAATCAAAGAGGCGAACTGTGTGTGAGAGGTCCT
ATGATTATGTCCGGTTATGTAAACAATCCCGAAGCGACCAACGCCTTGAT
TGACAAGGATGGATCGCTACATTCTGGAGACATAGCTTACTGGGACGAAG
ACGAACACTTCTTCATCGTTGACCGCCTGAAGTCTCTGATTAAGTACAAA
GGCTATCAGGTGGCTCCCGCTGAATTGGAATCCATCTTGCTCCAACACCC
CAACATCTTCGACGCAGGTGTCGCAGGTCTTCCCGACGATGACGCCCGTG
AACTTCCCCCCGCCGTTGTTGTTTTGGAGCACGGAAAGACGATGACGGAA
AAAGAGATCGTGGATTACGTCGCCAGTCAAGTAACAACCGCGAAAAAGTT
GCGCCCAGGAGTTGTGTTTGTGGACGAAGTACCGAAAGGTCTTACCGGAA
AACTCGACGCAAGAAAAATCACAGAGATCCTCATAAAGGCCAACAAGGGC
GGAAAGATCGCCGTGTAATTCTAGAGTCGGGGCGGCCCGCCCCTTCGAGC
AGACATGATAAGATACATTGATGAGTTTGGACAAACCACAACTAGAATGC
AGTGAAAAAAATGCTTTATTTGTGAAATTTGTGATGCTATTGCTTTATTT
GTAACCATTATAAGCTGCAATAAACAAGTTAACAACAACAATTGCATTCA
TTTTATGTTTCAGGTTCAGGCCCACCTGTGGGAGGTTTTTTAAAGCAAGT
AAAACCTCTACAAATGTGGTAAAATCGATAAGGATCGGCCGCAGGAACCC
CTAGTGATGGAGTTGGCCACTCCCTCTCTGCGCGCTCCCTCGCTCACTGA
CGCCGGGCGACCAAAGGTCCCCCGACCCCCGGCCTTTCCCCGGGCGGCCT
CACTCACCGACCGACCCCCCAGCTCCCTGCAGG

In the base sequence, the Left-ITR is the region represented by the following sequence:

(SEQ ID NO: 20)

CCTGCAGGCAGCTGCCCGCTCGCTCGCTCACTGAGGCCCCCCGGGCAAAG
CCCGGGCGTCGGGCGACCTTTGGTCCCCCCCCCTCAGTGAGCGAGCGAGC
GCGCAGAGAGGGACTGGCCAACTCCATCACTAGGGGTTCCT,

the 5′ flanking region of p21 gene is the region represented by the following sequence:

(SEQ ID NO: 21)

AAGCTTCCCAGCAACATGCTTGGGCAGCAGGCTGTGGCTCTCATTGCCTT
TCTGGCCCTCAGGAACATGTCCCAACATCTTGAGCTCTGGCATAGAAGAG
CCTGGTGGCTATTTTGTCCTTGGGCTCCCTGTTTTCAGGTGAGGAACGCC
ATGGTAGCAGACAGGAGACCTCTAAAGACCCCAGGTAAACCTTAGCCTGT
TACTCTGAACACCCTATGTGATCTGCCAGCAGATCCTTGCGACAGGGCTG
GGATCTGATGCATGTGTGCTTGTGTCAGTCTGTGCTGGGAGTCAGATTCT
GTGTGTGACTTTTAACAGCCTCCTCCCTTGCCTTTTTCACCCCAGAAGTC
CTCCCTTAGACTGTGTCTGGGTACACATTCAAGTGCATGGTTGCAAACTT
TTTTTTTTAAAGCACTGAATAGTACTAGACACTTAGTAGGTACTTAAGAA
ATATTGAATGTCGTGGTGGTGGTGAGCTAGAAGTTATAAAAAAAATTCTT
TCCCAAAAACAACAACAAAAAGAATTATTTCATTGTCAAGCTCAGTACCA
CAAAAATTTAAATAATTCATTACAAGCCTTTATTAAAAAAAATTTTCTCC
CCAAAGTAAACAGACAGACAATGTCTAGTCTATTTGAAATGCCTGAAAGC
AGAGGGGCTTCAAGGCAGTGGGAGAAGGTGCCTGTCCTCTGCTGGACATT
TGACAACCAGCCCTTTGGATGGTTTGGATCTATAGGAGCGAAGGTGCAGA
CAGCAGTGGGGCTTAGAGTGGGGTCCTGAGGCTGTGCCGTGGCCTTTCTG
GGGTTTAGCCACAATCCTGGCCTGACTCCAGGGCGAGGCAGGCCAAGGGG
GTCTGCTACTGTGTCCTCCCACCCCTACCTGGGCTCCCATCCCCACAGCA
GAGGAGAAAGAAGCCTGTCCTCCCCGAGGTCAGCTGCGTTACAGGAAGAA
GACTGGGCATGTCTGGGCAGACATTTCCAGACTCTGACCAGCCTGAGATG
TCAGTAATTGTAGCTGCTCCAAGCCTGGCTTCTCTTTTTTAGTGGGATTT
CTGTTCAGATGAACAATCCATCCTCTGCAATTTTTTAAAAGCAAAACTGC
AAATGTTTCAGGCACAGAAAGGAGCCAAAGGTGAAGTCCAGGGGAGGTCA
GGGGTGTCAGGTAGATGCCAGCGGATACACACATCACTCATTTCTGTGTC
TCTCAGAAGAACCAGTAGACACTTCCAGAATTGTCCTTTATTTATGTCAT
CTCCATAAACCATCTCCAAATGAGGGTTATTTGGCATTTTTCTCATTTTG
GAGCCACAGAAATAAAGGATGACAACCACAGAGCCCCGGGCAGGAGGCAA
AAGTCCTGTGTTCCAACTATAGTCATTTCTTTGCTGCATGATCTGAGTTA
CGTCACCAGACTTCTCTGAGCCCCAGTTTCCCCAGCAGTGTATACGGGCT
ATGTGCGGAGTATTCAGGAGACAGACAACTCACTCGTCAAATCCTCCCCT
TCCTGGCCAACAAACCTGCTGCAACCACAGGGATTTCTTCTGTTCAGGTG
AGTGTAGGGTGTAGGGAGATTGGTTCAATGTCCAATTCTTCTGTTTCCCT
GGAGATCAGGTTGCCCTTTTTTGGTACTCTCTCCAATTCCCTCCTTCCCG
GAAGCATGTGACAATCAACAACTTTGTATACTTAAGTTCAGTGGACCTCA
ATTTCCTCATCTGTGAAATAAACGGCACTGAAAAATCATTCTGGCCTCAA
GATGCTTTGTTGGGGTGTCTAGGTGCTCCAGGTGCTTCTGGGAGAGGTGA
CCTACTGACGGATCAGTCGCAATACACCTCATATTGTGCGGCTTTTCTGG
AAATTCCAGAGAGCTCCATCCTTTTTATAATTTATGAATTTTTATCTATT
AATCTCATCCTCCTCATCTTTTCAGCTGCATTCGGTAAATCCTTGCCTGC
CAGACTCGGTCAGCGGTGAGCCACAAAGCGGGCTCATTCTAACACTCCTG
TCTCCTCCTCCAGACTGCCAACTCATTCTCCAACTAAAAAAAGCCAGATT
TGTGCCTCACTTCCTCCCCAAATGTGTCCACCGCACCAACGCACCCGACG
GACTGGGGGAGGAGGGAAGTGCCCTCCTCCAGCACGCGAGGTTCCGCCAC
CGCCTGCCCTGCTCGAACTCCGCCACGCTCAGCTGGCTCGGCGCTGCCCA
CCCAGGACCCTGGCCCCCCCGGACCCCCCTCCCCCCCCGCGCGCTGGGCC
GACCCCCGCTCCCCCCTCCTTCAGGCGCCCCCGGGCGGGCCGCTTCTATA
TCACCGCCCCCCTGAGCTCCCCCAGCTCACGTGTCACCAGCTCCCCAAGT
CACTTCCTTGTCCAAGCTT,

the luciferase coding sequence is the region represented by the following sequence:

(SEQ ID NO: 22)

ATGGAACACGCCAAAAACATAAAGAAAGGCCCGGCGCCATTCTATCCGCT
GGAAGATCGAACCGGTGCACACCAACTGCATAACCCTATGAAGAGATACG
CCCTGGTTCCTGGAACAATTCCTTTTACAGATGCACATATCGAGCTCGAC
ATCACTTACGCTGAGTACTTCCAAATCTCCCTTCGGTTGGCAGAACCTAT
GAAACGATATGGGCTGAATACAAATCACACAATCGTCGTATGCAGTGAAA
ACTCTCTTCAATTCTTTATCCCCCTGTTGGGCCCGTTATTTATCGGAGTT
GCAGTTGCGCCCGCGAACGACATTTATAATGAACGTGAATTGCTCAACAG
TATGGGCATTTCGCAGCCTACCGTCGTGTTCGTTTCCAAAAAGCCGTTGC
AAAAAATTTTGAACGTGCAAAAAAACCTCCCAATCATCCAAAAAATTATT
ATCATCCATTCTAAAACGGATTACCAGGGATTTCAGTCGATGTACACGTT
CGTCACATCTCATCTACCTCCCGGTTTTAATGAATACGATTTTGTGCCAG
AGTCCTTCGATAGGCACAACACAATTGCACTCATCATGAACTCCTCTGGA
TCTACTGGTCTCCCTAAAGGTGTCGCTCTGCCTCATAGAACTGCCTCCCT
GAGATTCTCCCATGCCAGAGATCCTATTTTTGGCAATCAAATCATTCCCG
ATACTCCGATTTTAAGTGTTGTTCCATTCCATCACGGTTTTGGAATGTTT
ACTACACTCCGATATTTCATATCTGCATTTCCAGTCCTCTTAATGTATAC
ATTTGAAGAAGACCTGTTTCTCAGCACCCTTCACGATTACAACATTCAAA
GTGCCCTGCTCGTGCCAACCCTATTCTCCTTCTTCGCCAAAACCACTCTC
ATTGACAAATACGATTTATCTAATTTACACCAAATTCCTTCTGCTCCCCC
TCCCCTCTCTAACGAACTCGGGCAAGCCCTTCCCAACACCTTCCATCTCC
CAGCTATCACCCAACCATATCCCCTCACTCAGACTACATCAGCTATTCTC
ATTACACCCGAGGGGGATGATAAACCGGGCGCGGTCGGTAAAGTTGTTCC
ATTTTTTGAAGCCAAGGTTGTGGATCTGGATACCGGGAAAACGCTGCGCG
TTAATCAAAGAGCCGAACTGTGTCTGAGAGCTCCTATGATTATGTCCGGT
TATGTAAACAATCCGGAAGCGACCAACGCCTTGATTCACAAGGATGGATC
CCTACATTCTCGACACATAGCTTACTGGGACGAAGACGAACACTTCTTCA
TCGTTGACCGCCTGAAGTCTCTGATTAAGTACAAAGGCTATCAGGTGGCT
CCCGCTCAATTGGAATCCATCTTGCTCCAACACCCCAACATCTTCGACGC
AGGTGTCGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGATCGCCGTG
TAA,

the SV-40-derived polyadenylation signal is the region represented by the following sequence:

(SEQ ID NO: 23)

CAGACATGATAAGATACATTGATGAGTTTGGACAAACCACAACTAGAATG
CAGTGAAAAAAATCCTTTATTTGTGAAATTTCTCATGCTATTGCTTTATT
TGTAACCATTATAACCTCCAATAAACAACTTAACAACAACAATTGCATTC
ATTTTATGTTTCAGGTTCACGGGGACCTCTGGCAGCTTTTTTAAACCAAC
TAAAACCTCTACAAATGTGCTA,
and

the Right-ITR is the region represented by the following sequence:

AGGAACCCCTAGTGATGGAGTTGGCCACTCCCTCTCTGCGCGCTCGCTCGCTCACTGAGGCC GGGCGACCAAAGGTCGCCCGACGCCCGGGCTTTGCCCGGGCGGCCTCAGTGAGCGAGCGAGC GCGCAGCTGCCTGCAGG (SEQ ID NO: 24). Therefore, rAAV-PLS contained the DNA sequence comprising (a) the Left-ITR, (b) the p53 target gene promoter sequence, (c) the therapeutic gene sequence, (d) the polyadenylation signal sequence, and (e) the Right-ITR, in the order of (a), (d), (c), (b), (e) from the five prime end side to the three prime end side.

### Example 2

### Pharmaceutical Composition Comprising Integration-Type Low-Dose Radiation-Inducible Viral Vector

The low-dose radiation-inducible viral vector rAAV-PLS produced in 293 cells was recovered by four freeze-thaw cycles, then centrifuged at 10,000 g for 10 minutes and concentrated. The obtained concentrate contained the low-dose radiation-inducible viral vector rAAV-PLS and a buffer solution.

### Example 3

### Transduction Using Integration-Type Viral Vector

(1) Transduction

MCF-7 cells, which are human breast cancer cells expressing p53, were used as host cells.

MCF-7 cells were transduced with viral vector (multiplicity of infection: 5.5×10³) by mixing 0.25 ml of pharmaceutical composition prepared in Example 2 (viral inoculum) (containing 5.5×10⁸ rAAV-PLS viral particles) and 10⁵ MCF-7 cells in a 12-well microplate, and incubating for 24 hours (culturing in 2 ml of RPMI1640 containing 10% fetal bovine serum, in an atmosphere at 37° C. containing 5% carbon dioxide). Then, cells were washed with PBS to remove the viral inoculum and cultured in an RPMI1640 culture medium (Life Technologies) supplemented with 10% FBS (JRH), 100 unit/ml penicillin and 100 μg/ml streptomycin (Life Technologies), at 37° C., under 5% CO₂ in a humidified atmosphere.

(2) X-Ray Irradiation

MCF-7 cells cultured for 66 days after being transduced were irradiated with various doses of X-ray (0.2 Gy, 0.5 Gy, 1 Gy and 2 Gy). X-ray was generated from a Pantak unit fitted with a 0.5 mm copper filter and a 0.5 mm aluminum filter, and operating at 200 kVp and 20 mA. In addition, irradiation was carried out at a dose rate of 1.0 Gy/minute.

(3) Measurement of Luciferase Gene Expression

The expression of the transduced luciferase gene was evaluated with the amount of light emission generated in the MCF-7 cells by luciferase as an indicator.

After 6 hours X-ray irradiation, MCF-7 cells were washed with PBS, then lysed with Passive Lysis Buffer (Promega). The luciferase activity in the cell lysate was measured with an analytical luminometer (model LB9506; Berthold), using the Luciferase Assay System (Promega). Non-X-ray irradiated MCF-7 cells, for which the same treatments as the X-ray irradiated MCF-7 cells were carried out, except for the X-ray irradiation, were used as controls. From the obtained measurement values, the rate of induction of luciferase gene expression due to each exposure dose was calculated, based on the following calculation formula:
rate of induction=amount of light emission from X-ray-irradiated MCF-7 cells/amount of light emission from non-X-ray-irradiated MCF-7 cells

### Comparative Example 1

### Transfection Using Non-Integration-Type Plasmid Vector

(1) Transfection

The plasmid pLS, which is a non-integration-type plasmid vector generated in the creation step of the integration-type viral vector of the present invention, was used as a comparative example. Transfection into MCF-7 host cells was carried out according to the literature (Nenoi M, Ichimura S, Mita K, Yukawa O, Cartwright I L. Regulation of the catalase gene promoter by Sp1, CCAAT-recognizing factors, and a WT1/Egr-related factor in hydrogen peroxide-resistant HP100 cells. Cancer Res 2001; 61: 5885-5894), by the following procedure.

Washed with an RPMI1640 culture medium not containing FBS, 5×10⁶ MCF-7 cells were mixed with 10 μg of plasmid pLS. The mixture of MCF-7 cells and plasmid pLS was transferred to an electroporation cuvette having an inter-electrode spacing of 4 mm, left in ice for 5 minutes, then, using a capacitance of 960 μF, a pulse was applied at 220V. After incubating in ice for 10 minutes, the cells were suspended homogeneously in 48 ml of preheated RPMI1640 containing FBS and incubated until X-ray irradiation (cultured in 2 ml of RPMI1640 containing 10% fetal bovine serum, in an atmosphere at 37° C. containing 5% carbon dioxide).

(2) X-Ray Irradiation

MCF-7 cells cultured for 48 hours after transfection were irradiated with various doses of X-ray (0.5 Gy, 1 Gy, 2 Gy, 3 Gy and 5 Gy). X-ray was generated from a Pantak unit fitted with a 0.5 mm copper filter and a 0.5 mm aluminum filter, and operating at 200 kVp and 20 mA. In addition, irradiation was carried out at a dose rate of 1.0 Gy/minute.

(3) Measurement of Luciferase Gene Expression

The expression of the transfected luciferase gene was evaluated with the amount of light emission generated in the MCF-7 cells by luciferase as an indicator.

After 48 hours X-ray irradiation, MCF-7 cells were washed with PBS, then lysed with Passive Lysis Buffer (Promega). The luciferase activity in the cell lysate was measured with an analytical luminometer (model LB9506; Berthold), using the Luciferase Assay System (Promega). Non-X-ray irradiated MCF-7 cells, for which the same treatments as the X-ray irradiated MCF-7 cells were carried out, except for the X-ray irradiation, were used as controls. From the obtained measurement values, the rate of induction of luciferase gene expression at each exposure dose was calculated, based on the following calculation formula:
rate of induction=amount of light emission from X-ray-irradiated MCF-7 cells/amount of light emission from non-X-ray-irradiated MCF-7 cells

Comparison Between Integration-Type Viral Vector and Non-Integration-Type Plasmid Vector Regarding the Rate of Radiation Induction of Luciferase Gene Expression

The dose dependency of the rate of induction of luciferase gene expression obtained in Example 3 and Comparative Example 1 is shown in FIG. 2.

When 0.5 Gy, 1 Gy, 2 Gy, 3 Gy and 5 Gy X-rays were irradiated, the non-integration-type plasmid vectors of Comparative Example 1 showed inductions of the luciferase gene expression, respectively, 1.1-times (10%), 1.3-times (30%), 1.4-times (40%) and 1.5-times (50%) than those of the luciferase gene expression without irradiation (the numbers between parentheses indicate the rates of increase when compared to the expression activity without irradiation). This result indicates that, under low-dose irradiation, the non-integration-type plasmid vector is unable to induce the therapeutic gene expression sufficiently.

On the other hand, when X-rays of 0.2 Gy, 0.5 Gy, 1 Gy and 2 Gy, were irradiated, the integration-type viral vector (adeno-associated virus vector) of the present invention showed inductions of the luciferase gene expression, respectively, 1.3-times (30%), 1.7-times (70%), 2.1-times (110%) and 3.1-times (210%) than those of the luciferase gene expression without irradiation (the numbers between parentheses indicate the rates of increase when compared to the expression activity without irradiation). This result indicates that, under low-dose irradiation, the integration-type viral vector of the present invention is capable of inducing therapeutic gene expression sufficiently.

Herein, Example 3 and Comparative Example 1 differ in the duration of culture from after gene introduction until X-ray irradiation (Example: 66 days after transduction; Comparative Example: 48 hours after transfection). However, (1) as the transgenes used in Example 3 and Comparative Example 1 do not contain factors that are thought to influence the ordinary physiological state of cells, and (2) as a long time period elapsed from transgenic manipulations such that transient cell alterations are over, the difference in the duration of culture from after gene introduction until X-ray irradiation is not thought to influence the rate of radiation induction.

Regarding this result, although the present invention is not to be confined to a specific theory, considering that the target gene expression activation by p53 activated by low-dose irradiation is related to a mechanism depending on a high order chromosome structure, it is thought that the viral vector of the present invention has a low-dose radiation-inducibility owing to the presence of p53 target gene promoter sequence and therapeutic gene sequence in an integrated state in the chromosome of the host cell due to the integration-type viral vector of the present invention.

From the above, it is understood that the integration-type viral vector of the present invention has low-dose radiation-inducibility.

### Reference Example

### Transgenic State in MCF-7 Cells Transduced with rAAV-PLS

The following two experiments were carried out in order to verify that the integration-type viral vector of the present invention can give rise to gene integration into the host chromosome.

(1) Southern Blot Analysis of Transduced MCF-7 Cell Genomic DNA Using rAAV-PLS-Specific Primers

According to the transduction method of Example 3 (1), MCF-7 cells were transduced with the integration-type low-dose radiation-inducible viral vector rAAV-PLS created in Example 1.

From MCF-7 cells cultured for 66 days after transduction, the MCF-7 cell genomic DNA was isolated using DNAzol (Invitrogen). The isolated genomic DNA was amplified by PCR (reaction condition: 1 minute at 95° C., 30 seconds at 60° C. and 1 minute at 72° C.) using two species of rAAV-PLS-specific primers having the following sequences:

TCCTGGAGAGTGCCAACTCATTCTC (SEQ ID NO: 25) and

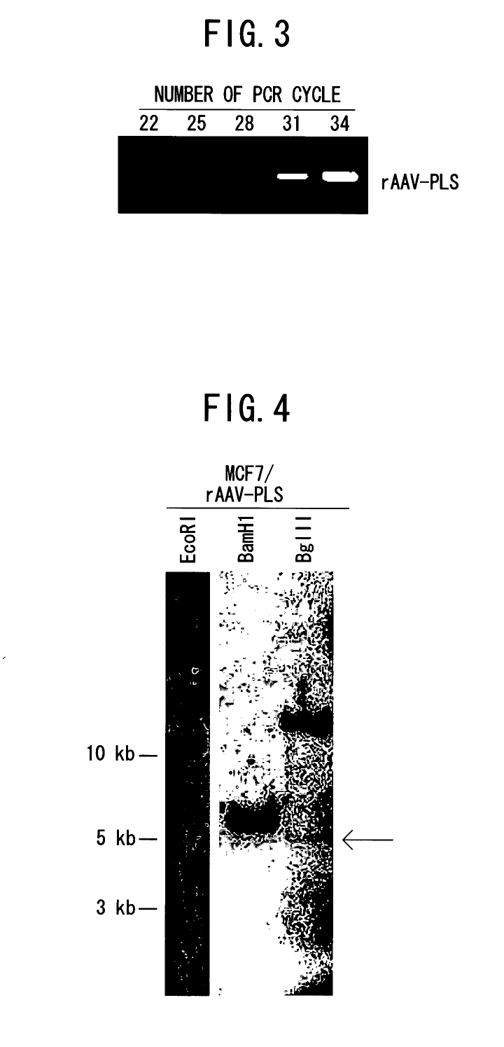

TTCCAGGAACCAGGGCGTATCTCTTC (SEQ ID NO: 26), LA Taq polymerase (Takara Shuzo) and GC buffer (Takara Shuzo). The result is shown in FIG. 3.

FIG. 3 shows that, resulting from subjecting, as the template, the genomic DNA isolated from MCF-7 cells transduced with rAAV-PLS to 31 or more PCR cycles, a product specific to the DNA sequence of rAAV-PLS appeared as a clear band. From this result, it is understood that, in the genomic DNA sequence isolated from the MCF-7 cells transduced with rAAV-PLS, a sequence corresponding to a portion of the rAAV-PLS is present.

(2) Southern Blot Analysis of Transduced MCF-7 Cell Genomic DNA Using Restriction Enzymes

The present experiment was carried out to verify that the DNA of rAAV-PLS was actually integrated into the chromosome of MCF-7 cells.

According to the transduction method of Example 3 (1), MCF-7 cells were transduced with the integration-type low-dose radiation-inducible viral vector rAAV-PLS created in Example 1.

From MCF-7 cells cultured for 66 days after transduction, the MCF-7 cell genomic DNA was isolated using DNAzol (Invitrogen). The isolated genomic DNA was digested with any one of three restriction enzymes: BglII, EcoRI and BamH1.

Herein, BglII is a restriction enzyme that is capable of cutting not only near the five prime end of the p21 promoter in the rAAV-PLS genome, but also a region near the five prime end of the p21 promoter in the genomic DNA of MCF-7 cells that is inherent to MCF-7 cells. On the other hand, EcoRI and BamH1 are restriction enzymes that can cut the genomic DNA of MCF-7 cells, but cannot cut the rAAV-PLS genome.

The respective digestion products obtained using each species of restriction enzyme were subjected to electrophoresis in a 0.7% agarose gel and blotted onto a GeneScreen nylon filter.

The filter and a probe having the following sequence corresponding to a potion of the DNA sequence of rAAV-PLS (a portion of luciferase coding sequence and p21 gene promoter sequence) were hybridized:

AGTACTAGACACTTAGTAGGTACTTAAGAAATATTGAATGTCGTGGTGGTGGTGAGCTAGAA GTTATAAAAAAAATTCTTTCCCAAAAACAACAACAAAAAGAATTATTTCATTGTGAAGCTCA GTACCACAAAAATTTAAATAATTCATTACAAGCCTTTATTAAAAAAAATTTTCTCCCCAAAG TAAACAGACAGACAATGTCTAGTCTATTTGAAATGCCTGAAAGCAGAGGGGCTTCAAGGCAG TGGGAGAAGGTGCCTGTCCTCTGCTGGACATTTGACAACCAGCCCTTTGGATGGTTTGGATG TATAGGAGCGAAGGTGCAGACAGCAGTGGGGCTTAGAGTGGGGTCCTGAGGCTGTGCCGTGG CCTTTCTGGGGTTTAGCCACAATCCTGGCCTGACTCCAGGGCGAGGCAGGCCAAGGGGGTCT GCTACTGTGTCCTCCCACCCCTACCTGGGCTCCCATCCCCACAGCAGAGGAGAAAGAAGCCT GTCCTCCCCGAGGTCAGCTGCGTTAGAGGAAGAAGACTGGGCATGTCTGGGCAGAGATTTCC AGACTCTGAGCAGCCTGAGATGTCAGTAATTGTAGCTGCTCCAAGCCTGGGTTCTGTTTTTT AGTGGGATTTCTGTTCAGATGAACAATCCATCCTCTGCAATTTTTTAAAAGCAAAACTGCAA ATGTTTCAGGCACAGAAAGGAGGCAAAGGTGAAGTCCAGGGGAGGTCAGGGGTGTGAGGTAG ATGGGAGCGGATAGACACATCACTCATTTCTGTGTCTGTCAGAAGAACCAGTAGACACTTCC AGAATTGTCCTTTATTTATGTCATCTCCATAAACCATCTGCAAATGAGGGTTATTTGGCATT TTTGTCATTTTGGAGCCACAGAAATAAAGGATGACAAGCAGAGAGCCCCGGGCAGGAGGCAA AAGTCCTGTGTTCCAACTATAGTCATTTCTTTGCTGCATGATCTGAGTTAGGTCACCAGACT TCTCTGAGCCCCAGTTTCCCCAGCAGTGTATACGGGCTATGTGGGGAGTATTCAGGAGACAG ACAACTCACTCGTCAAATCCTCCCCTTCCTGGCCAACAAAGCTGCTGCAACCACAGGGATTT CTTCTGTTCAGGTGAGTGTAGGGTGTAGGGAGATTGGTTCAATGTCCAATTCTTCTGTTTCC CTGGAGATCAGGTTGCCCTTTTTTGGTAGTCTCTCCAATTCCCTCCTTCCCGGAAGCATGTG ACAATCAACAACTTTGTATACTTAAGTTCAGTGGACCTCAATTTCCTCATCTGTGAAATAAA CGGGACTGAAAAATCATTCTGGCCTCAAGATGCTTTGTTGGGGTGTCTAGGTGCTCCAGGTG CTTCTGGGAGAGGTGACCTAGTGAGGGATCAGTGGGAATAGAGGTGATATTGTGGGGCTTTT CTGGAAATTGCAGAGAGGTGCATCGTTTTTATAATTTATGAATTTTTATGTATTAATGTCAT CCTCCTGATCTTTTCAGCTGCATTGGGTAAATCCTTGCCTGCCAGAGTGGGTCAGCGGTGAG CCAGAAAGGGGGCTCATTCTAACAGTGCTGTGTCCTCCTGGAGAGTGCCAACTCATTCTCCA AGTAAAAAAAGCCAGATTTGTGGCTCACTTCGTGGGGAAATGTGTCCAGCGCACCAACGCAG GCGAGGGACTGGGGGAGGAGGGAAGTGCCCTCCTGCAGCACGCGAGGTTCCGGGACCGGCTG GCCTGCTGGAACTCGGCCAGGCTCAGCTGGCTCGGCGCTGGGCAGCCAGGAGCCTGGGCCCC GGGGAGGGCGGTCCCGGGCGGCGCGGTGGGCCGAGCGCGGGTCCCGCCTCCTTGAGGCGGGC CCGGGCGGGGCGGTTGTATATCAGGGCCGCGCTGAGCTGCGCCAGCTGAGGTGTGAGCAGCT GCCGAAGTCAGTTCCTTGTGGAAGCTTGGCATTCCGGTACTGTTGGTAAAGCCACCATGGAA GACGCCAAAAACATAAAGAAAGGCCCGGCGCCATTCTATCCGCTGGAAGATGGAACCGCTGG AGAGCAACTGCATAAGGCTATGAAGAGATACGCCCTGGTTCCTGGAACAATTGCTTTTACAG ATGCACATATCGAGGTGGACATCACTTACGCTGAGTACT (SEQ ID NO: 27). The hybridization signal intensity was measured with the BAS2000 Bio-Imaging Analyzer (Fuji Film). The result is shown in FIG. 4.

When the genomic DNA of transduced MCF-7 cells was digested with BglII, two bands were observed at >10 kb position and 5 kb position.

The >10 kb band is thought to correspond to a fragment comprising the p21 gene promoter region inherent to MCF-7 cells. The 5 kb band is thought to correspond to a fragment generated by integrating rAAV-PLS's into the MCF-7 cell genome, in a state where they are linked in tandem, and cutting at the BglII restriction site within the p21 promoter region present in the DNA sequence of each rAAV-PLS.

When the genomic DNA of the transduced MCF-7 cells was digested with EcoRI, a band was observed at >10 kb position. This band is thought to correspond to a fragment comprising the p21 gene promoter region inherent to MCF-7 cells.

When the genomic DNA of the transduced MCF-7 cells was digested with BamH1, a band was observed at a >5 kb position. This band is thought to correspond to a fragment comprising the p21 gene promoter region inherent to MCF-7 cells.

If the DNA of rAAV-PLS introduced into MCF-7 cells were present outside the chromosomes of MCF-7 cells, then, when digesting with EcoRI or BamH1, which are unable to cut the rAAV-PLS genome, a band should have appeared with a size identical with that of a band corresponding to the intact DNA of rAAV-PLS. However, such a band was not observed. Therefore, the DNA of rAAV-PLS is thought to be integrated in the chromosome of MCF-7 cells.

Regarding the state of integration into the chromosome of the DNA of rAAV-PLS, if the DNA of rAAV-PLS is integrated at a specific position of the MCF-7 cell chromosome, then, as a DNA fragment with a defined length (comprising the DNA of rAAV-PLS) is generated after digestion with EcoRI or BamH1, a band corresponding to this fragment should appear in a Southern blot analysis. On the other hand, if the DNA of rAAV-PLS is integrated randomly in the of MCF-7 cell chromosome, then, as DNA fragments with various (undefined) lengths (comprising the DNA of rAAV-PLS) are generated after digestion with EcoRI or BamH1, they would not appear as a band in a Southern blot analysis, and should appear as a smear. In the above experimental result, only a band corresponding to a fragment comprising the p21 gene promoter region inherent to MCF-7 cells was observed. Therefore, the DNA of rAAV-PLS is thought to be integrated randomly in the MCF-7 cell chromosome.

### Example 4

### Integration-Type Low-Dose Radiation-Inducible Viral Vector

In the present example, a low-dose radiation-inducible viral vector rAAV-PtkS was constructed based on a type 2 adeno-associated virus, having the p21 gene promoter sequence as the p53 target gene promoter sequence, and having herpes simplex virus thymidine kinase (HSV-tk) gene sequence as the therapeutic gene sequence. rAAV-PtkS was constructed by the triple transfection method (Xiao X, Li J, Samulski R J. Production of high-titer recombinant adeno-associated virus vectors in the absence of helper adenovirus. J Virol 1998; 72: 2224-2232. and Matsushita T, Elliger S, Elliger C, Podsakoff G, Villarreal L, Kurtzman G J, Iwaki Y, Colosi P. Adeno-associated virus vectors can be efficiently produced without helper virus. Gene Ther 1998; 5: 938-945.) using the AAV Helper Free System (Stratagene).

First, the fragment:

CCATGGCCTCGTACCCCGGCCATCAACACGCGTCTGCGTTCGACCAGGCTGCGCGTTCTCGC GGCCATAGCAACCGACGTACGGCGTTGCGCCCTCGCCGGCAGCAAGAAGCCACGGAAGTCCG CCCGGAGCAGAAAATGCCCACGCTACTGCGGGTTTATATAGACGGTCCCCACGGGATGGGGA AAACCACCACCACGCAACTGCTGGTGGCCCTGGGTTCGCGCGACGATATCGTCTACGTACCC GAGCCGATGACTTACTGGCGGGTGCTGGGGGCTTCCGAGACAATCGCGAACATCTACACCAC ACAACACCGCCTCGACCAGGGTGAGATATCGGCCGGGGACGCGGCGGTGGTAATGACAAGCG CCCAGATAACAATGGGCATGCCTTATGCCGTGACCGACGCCGTTCTGGCTCCTCATATCGGG GGGGAGGCTGGGAGCTCACATGCCCCGCCCCCGGCCCTCACCCTCATCTTCGACCGCCATCC CATCGCCGCCCTCCTGTGCTACCCGGCCGCGCGGTACCTTATGGGCAGCATGACCCCCCAGG CCGTGCTGGCGTTCGTGGCCCTCATCCCGCCGACCTTGCCCGGCACCAACATCGTGCTTGGG GCCCTTCCGGAGGACAGACACATCGACCGCCTGGCCAAACGCCAGCGCCCCGGCGAGCGGCT GGACCTGGCTATGCTGGCTGCGATTCGCCGCGTTTACGGGCTACTTGCCAATACGGTGCGGT ATCTGCAGTGCGGCGGGTCGTGGCGGGAGGACTGGGGACAGCTTTCGGGGACGGCCGTGCCG CCCCAGGGTGCCGAGCCCCAGAGCAACGCGGGCCCACGACCCCATATCGGGGACACGTTATT TACCCTGTTTCGGGCCCCCGAGTTGCTGGCCCCCAACGGCGACCTGTATAACGTGTTTGCCT GGGCCTTGGACGTCTTGGCCAAACGCCTCCGTTCCATGCACGTCTTTATCCTGGATTACGAC CAATCGCCCGCCGGCTGCCGGGACGCCCTGCTGCAACTTACCTCCGGGATGGTCCAGACCCA CGTCACCACCCCCGGCTCCATACCGACGATATGCGACCTGGCGCGCACGTTTGCCCGGGAGA TGGGGGAGGCTAACTGAGAATTCGCTAGC (SEQ ID NO: 28) comprising the HSV-tk coding sequence was excised from plasmid pORF-HSVtk (InvivoGen) using NcoI and NheI.

Meanwhile, using NcoI and XbaI, a fragment comprising Left-ITR, 5′ flanking region of the p21 gene, SV40-derived polyadenylation signal, and Right-ITR was excised from pAAV-PLS having the following sequence:

(SEQ ID NO:29)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCTCG
AGATCTGCGATCTAAGTAAGCTTCCCAGGAACATGCTTGGGCAGCAGGCT
GTGGCTCTGATTGGCTTTCTGGCCGTCAGGAACATGTCCCAACATGTTGA
GCTCTGGCATAGAAGAGGCTGGTGGCTATTTTGTCCTTGGGCTGCCTGTT
TTCAGGTGAGGAAGGGGATGGTAGGAGACAGGAGACCTCTAAAGACCCCA
GGTAAACCTTAGCCTGTTACTCTGAACAGGGTATGTGATCTGCCAGCAGA
TCCTTGCGACAGGGCTGGGATCTGATGCATGTGTGCTTGTGTGAGTGTGT
GCTGGGAGTCAGATTCTGTGTGTGACTTTTAACAGCCTGCTCCCTTGCCT
TTTTCAGGGCAGAAGTCCTCCCTTAGAGTGTGTCTGGGTACACATTCAAG
TGCATGGTTGCAAACTTTTTTTTTTAAAGCACTGAATAGTACTAGACACT
TAGTAGGTACTTAAGAAATATTGAATGTCGTGGTGGTGGTGAGCTAGAAG
TTATAAAAAAAATTCTTTCCCAAAAACAACAACAAAAAGAATTATTTCAT
TGTGAAGCTCAGTACCACAAAAATTTAAATAATTCATTACAAGCCTTTAT
TAAAAAAAATTTTCTCCCCAAAGTAAACAGACAGACAATGTCTAGTCTAT
TTGAAATGCCTGAAAGCAGAGGGGCTTCAAGGCAGTGGGAGAAGGTGCCT
GTCCTCTGCTGGACATTTGACAACCAGCCCTTTGGATGGTTTGGATGTAT
AGGAGCGAAGGTGCAGACAGCAGTGGGGCTTAGAGTGGGGTCCTGAGGCT
GTGCCGTGGCCTTTCTGGGGTTTAGCCACAATCCTGGCCTGACTCCAGGG
CGAGGCAGGCCAAGGGGGTCTGCTACTGTGTCCTCCCACCCCTACCTGGG
CTCCCATCCCCACAGCAGAGGAGAAAGAAGCCTGTCCTCCCCGAGGTCAG
CTGCGTTAGAGGAAGAAGACTGGGCATGTCTGGGCAGAGATTTCCAGACT
CTGAGCAGCCTGAGATGTCAGTAATTGTAGCTGCTCCAAGCCTGGGTTCT
GTTTTTTAGTGGGATTTCTGTTCAGATGAACAATCCATCCTCTGCAATTT
TTTAAAAGCAAAACTGCAAATGTTTCAGGCACAGAAAGGAGGCAAAGGTG
AAGTCCAGGGGAGGTCAGGGGTGTGAGGTAGATGGGAGCGGATAGACACA
TCACTCATTTCTGTGTCTGTCAGAAGAACCAGTAGACACTTCCAGAATTG
TCCTTTATTTATGTCATCTCCATAAACCATCTGCAAATGAGGGTTATTTG
GCATTTTTGTCATTTTGGAGCCACAGAAATAAAGGATGACAAGCAGAGAG
CCCCGGGCAGGAGGCAAAAGTCCTGTGTTCCAACTATAGTCATTTCTTTG
CTGCATGATCTGAGTTAGGTCACCAGACTTCTCTGAGCCCCAGTTTCCCC
AGCAGTGTATACGGGCTATGTGGGGAGTATTCAGGAGACAGACAACTCAC
TCGTCAAATCCTCCCCTTCCTGGCCAACAAAGCTGCTGCAACCACAGGGA
TTTCTTCTGTTCAGGTGAGTGTAGGGTGTAGGGAGATTGGTTCAATGTCC
AATTCTTCTGTTTCCCTGGAGATCAGGTTGCCCTTTTTTGGTAGTCTCTC
CAATTCCCTCCTTCCCGGAAGCATGTGACAATCAACAACTTTGTATACTT
AAGTTCAGTGGACCTCAATTTCCTCATCTGTGAAATAAACGGGACTGAAA
AATCATTCTGGCCTCAAGATGCTTTGTTGGGGTGTCTAGGTGCTCCAGGT
GCTTCTGGGAGAGGTGACCTAGTGAGGGATCAGTGGGAATAGAGGTGATA
TTGTGGGGCTTTTCTGGAAATTGCAGAGAGGTGCATCGTTTTTATAATTT
ATGAATTTTTATGTATTAATGTCATCCTCCTGATCTTTTCAGCTGCATTG
GGTAAATCCTTGCCTGCCAGAGTGGGTCAGCGGTGAGCCAGAAAGGGGGC
TCATTCTAACAGTGCTGTGTCCTCCTGGAGAGTGCCAACTCATTCTCCAA
GTAAAAAAAGCCAGATTTGTGGCTCACTTCGTGGGGAAATGTGTCCAGCG
CACCAACGCAGGCGAGGGACTGGGGGAGGAGGGAAGTGCCCTCCTGCAGC
ACGCGAGGTTCCGGGACCGGCTGGCCTGCTGGAACTCGGCCAGGCTCAGC
TGGCTCGGCGCTGGGCAGCCAGGAGCCTGGGCCCCGGGGAGGGCGGTCCC
GGGCGGCGCGGTGGGCCGAGCGCGGGTCCCGCCTCCTTGAGGCGGGCCCG
GGCGGGGCGGTTGTATATCAGGGCCGCGCTGAGCTGCGCCAGCTGAGGTG
TGAGCAGCTGCCGAAGTCAGTTCCTTGTGGAAGCTTGGCATTCCGGTACT
GTTGGTAAAGCCACCATGGAAGACGCCAAAAACATAAAGAAAGGCCCGGC
GCCATTCTATCCGCTGGAAGATGGAACCGCTGGAGAGCAACTGCATAAGG
CTATGAAGAGATACGCCCTGGTTCCTGGAACAATTGCTTTTACAGATGCA
CATATCGAGGTGGACATCACTTACGCTGAGTACTTCGAAATGTCCGTTCG
GTTGGCAGAAGCTATGAAACGATATGGGCTGAATACAAATCACAGAATCG
TCGTATGCAGTGAAAACTCTCTTCAATTCTTTATGCCGGTGTTGGGCGCG
TTATTTATCGGAGTTGCAGTTGCGCCCGCGAACGACATTTATAATGAACG
TGAATTGCTCAACAGTATGGGCATTTCGCAGCCTACCGTGGTGTTCGTTT
CCAAAAAGGGGTTGCAAAAAATTTTGAACGTGCAAAAAAAGCTCCCAATC
ATCCAAAAAATTATTATCATGGATTCTAAAACGGATTACCAGGGATTTCA
GTCGATGTACACGTTCGTCACATCTCATCTACCTCCCGGTTTTAATGAAT
ACGATTTTGTGCCAGAGTCCTTCGATAGGGACAAGACAATTGCACTGATC
ATGAACTCCTCTGGATCTACTGGTCTGCCTAAAGGTGTCGCTCTGCCTCA
TAGAACTGCCTGCGTGAGATTCTCGCATGCCAGAGATCCTATTTTTGGCA
ATCAAATCATTCCGGATACTGCGATTTTAAGTGTTGTTCCATTCCATCAC
GGTTTTGGAATGTTTACTACACTCGGATATTTGATATGTGGATTTCGAGT
CGTCTTAATGTATAGATTTGAAGAAGAGCTGTTTCTGAGGAGCCTTCAGG
ATTACAAGATTCAAAGTGCGCTGCTGGTGCCAACCCTATTCTCCTTCTTC
GCCAAAAGCACTCTGATTGACAAATACGATTTATCTAATTTACACGAAAT
TGCTTCTGGTGGCGCTCCCCTCTCTAAGGAAGTCGGGGAAGCGGTTGCCA
AGAGGTTCCATCTGCCAGGTATCAGGCAAGGATATGGGCTCACTGAGACT
ACATCAGCTATTCTGATTACACCCGAGGGGGATGATAAACCGGGCGCGGT
CGGTAAAGTTGTTCCATTTTTTGAAGCGAAGGTTGTGGATCTGGATACCG
GGAAAACGCTGGGCGTTAATCAAAGAGGCGAACTGTGTGTGAGAGGTCCT
ATGATTATGTCCGGTTATGTAAACAATCCGGAAGCGACCAACGCCTTGAT
TGACAAGGATGGATGGCTACATTCTGGAGACATAGCTTACTGGGACGAAG
ACGAACACTTCTTCATCGTTGACCGCCTGAAGTCTCTGATTAAGTACAAA
GGCTATCAGGTGGCTCCCGCTGAATTGGAATCCATCTTGCTCCAACACCC
CAACATCTTCGACGCAGGTGTCGCAGGTCTTCCCGACGATGACGCCGGTG
AACTTCCCGCCGCCGTTGTTGTTTTGGAGCACGGAAAGACGATGACGGAA
AAAGAGATCGTGGATTACGTCGCCAGTCAAGTAACAACCGCGAAAAAGTT
GCGCGGAGGAGTTGTGTTTGTGGACGAAGTACCGAAAGGTCTTACCGGAA
AACTCGACGCAAGAAAAATCAGAGAGATCCTCATAAAGGCCAAGAAGGGC
GGAAAGATCGCCGTGTAATTCTAGAGTCGGGGCGGCCGGCCGCTTCGAGC
AGACATGATAAGATACATTGATGAGTTTGGACAAACCACAACTAGAATGC
AGTGAAAAAAATGCTTTATTTGTGAAATTTGTGATGCTATTGCTTTATTT
GTAACCATTATAAGCTGCAATAAACAAGTTAACAACAACAATTGCATTCA
TTTTATGTTTCAGGTTCAGGGGGAGGTGTGGGAGGTTTTTTAAAGCAAGT
AAAACCTCTACAAATGTGGTAAAATCGATAAGGATCGGCCGCAGGAACCC
CTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCT
CAGTGAGCGAGCGAGCGCGCAGCTGCCTGCAGGGGCGCCTGATGCGGTAT
TTTCTCCTTACGCATCTGTGCGGTATTTCACACCGCATACGTCAAAGCAA
CCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCA
CCTCGACCCCAAAAAACTTGATTTGGGTGATGGTTCACGTAGTGGGCCAT
CGCCCTGATAGACGGTTTTTCGCCCTTTGACGTTGGAGTCCACGTTCTTT
AATAGTGGACTCTTGTTCCAAACTGGAACAACACTCAACCCTATCTCGGG
CTATTCTTTTGATTTATAAGGGATTTTGCCGATTTCGGCCTATTGGTTAA
AAAATGAGCTGATTTAACAAAAATTTAACGCGAATTTTAACAAAATATTA
ACGTTTACAATTTTATGGTGCACTCTCAGTACAATCTGCTCTGATGCCGC
ATAGTTAAGCCAGCCCCGACACCCGCCAACACCCGCTGACGCGCCCTGAC
GGGCTTGTCTGCTCCCGGCATCCGCTTACACAGAAGCTGTGACCGTCTCC
GGGAGCTGCATGTGTCAGAGGTTTTCACCGTCATCACCGAAACGCGCGAG
ACGAAAGGGCCTCGTGATACGCCTATTTTTATAGGTTAATGTCATGATAA
TAATGGTTTCTTAGACGTCAGGTGGCACTTTTCGGGGAAATGTGCGCGGA
ACCCCTATTTGTTTATTTTTCTAAATACATTCAAATATGTATCCGCTCAT
GAGACAATAACCCTGATAAATGCTTCAATAATATTGAAAAAGGAAGAGTA
TGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGC
TGAAGATCAGTTGGGTGCACGAGTGGGTTACATCGAACTGGATCTCAACA
GCGGTAAGATCCTTGAGAGTTTTCGCCCCGAAGAACGTTTTCCAATGATG
AGCACTTTTAAAGTTCTGCTATGTGGCGCGGTATTATCCCGTATTGACGC
CGGGCAAGAGCAACTCGGTCGCCGCATACACTATTCTCAGAATGACTTGG
TTGAGTACTCACCAGTCACAGAAAAGCATCTTACGGATGGCATGACAGTA
AGAGAATTATGCAGTGCTGCCATAACCATGAGTGATAACACTGCGGCCAA
CTTACTTCTGACAACGATCGGAGGACCGAAGGAGCTAACCGCTTTTTTGC
ACAACATGGGGGATCATGTAACTCGCCTTGATCGTTGGGAACCGGAGCTG
AATGAAGCCATACCAAACGACGAGCGTGACACCACGATGCCTGTAGCAAT
GGCAACAACGTTGCGCAAACTATTAACTGGCGAACTACTTACTCTAGCTT
CCCGGCAACAATTAATAGACTGGATGGAGGCGGATAAAGTTGCAGGACCA
CTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATG
GTAAGCCCTCCCGTATCGTAGTTATCTACACGACGGGGAGTCAGGCAACT
ATGGATGAACGAAATAGACAGATCGCTGAGATAGGTGCCTCACTGATTAA
GCATTGGTAACTGTCAGACCAAGTTTACTCATATATACTTTAGATTGATT
TAAAACTTCATTTTTAATTTAAAAGGATCTAGGTGAAGATCCTTTTTGAT
AATCTCATGACCAAAATCCCTTAACGTGAGTTTTCGTTCCACTGAGCGTC
AGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTT
CAGCAGAGCGCAGATACCAAATACTGTCCTTCTAGTGTAGCCGTAGTTAG
GCCACCACTTCAAGAACTCTGTAGCACCGCCTACATACCTCGCTCTGCTA
ATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAA
CGGGGGGTTCGTGCACACAGCCCAGCTTGGAGCGAACGACCTACACCGAA
CTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCC
TGGCCTTTTGCTGGCCTTTTGCTCACATGT

and the fragment comprising the above HSV-tk coding sequence was ligated. Thus was pAAV-PtkS obtained, comprising Left-ITR, the 5′ flanking region of the p21 gene, HSV-tk coding sequence, SV40-derived polyadenylation signal, and Right-ITR ligated in this order.

The base sequence of pAAV-PtkS is shown below.

(SEQ ID NO:30)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCTCG
AGATCTGCGATCTAAGTAAGCTTCCCAGGAACATGCTTGGGCAGCAGGCT
GTGGCTCTGATTGGCTTTCTGGCCGTCAGGAACATGTCCCAACATGTTGA
GCTCTGGCATAGAAGAGGCTGGTGGCTATTTTGTCCTTGGGCTGCCTGTT
TTCAGGTGAGGAAGGGGATGGTAGGAGACAGGAGACCTCTAAAGACCCCA
GGTAAACCTTAGCCTGTTACTCTGAACAGGGTATGTGATCTGCCAGCAGA
TCCTTGCGACAGGGCTGGGATCTGATGCATGTGTGCTTGTGTGAGTGTGT
GCTGGGAGTCAGATTCTGTGTGTGACTTTTAACAGCCTGCTCCCTTGCCT
TTTTCAGGGCAGAAGTCCTCCCTTAGAGTGTGTCTGGGTACACATTCAAG
TGCATGGTTGCAAACTTTTTTTTTTAAAGCACTGAATAGTACTAGACACT
TAGTAGGTACTTAAGAAATATTGAATGTCGTGGTGGTGGTGAGCTAGAAG
TTATAAAAAAAATTCTTTCCCAAAAACAACAACAAAAAGAATTATTTCAT
TGTGAAGCTCAGTACCACAAAAATTTAAATAATTCATTACAAGCCTTTAT
TAAAAAAAATTTTCTCCCCAAAGTAAACAGACAGACAATGTCTAGTCTAT
TTGAAATGCCTGAAAGCAGAGGGGCTTCAAGGCAGTGGGAGAAGGTGCCT
GTCCTCTGCTGGACATTTGACAACCAGCCCTTTGGATGGTTTGGATGTAT
AGGAGCGAAGGTGCAGACAGCAGTGGGGCTTAGAGTGGGGTCCTGAGGCT
GTGCCGTGGCCTTTCTGGGGTTTAGCCACAATCCTGGCCTGACTCCAGGG
CGAGGCAGGCCAAGGGGGTCTGCTACTGTGTCCTCCCACCCCTACCTGGG
CTCCCATCCCCACAGCAGAGGAGAAAGAAGCCTGTCCTCCCCGAGGTCAG
CTGCGTTAGAGGAAGAAGACTGGGCATGTCTGGGCAGAGATTTCCAGACT
CTGAGCAGCCTGAGATGTCAGTAATTGTAGCTGCTCCAAGCCTGGGTTCT
GTTTTTTAGTGGGATTTCTGTTCAGATGAACAATCCATCCTCTGCAATTT
TTTAAAAGCAAAACTGCAAATGTTTCAGGCACAGAAAGGAGGCAAAGGTG
AAGTCCAGGGGAGGTCAGGGGTGTGAGGTAGATGGGAGCGGATAGACACA
TCACTCATTTCTGTGTCTGTCAGAAGAACCAGTAGACACTTCCAGAATTG
TCCTTTATTTATGTCATCTCCATAAACCATCTGCAAATGAGGGTTATTTG
GCATTTTTGTCATTTTGGAGCCACAGAAATAAAGGATGACAAGCAGAGAG
CCCCGGGCAGGAGGCAAAAGTCCTGTGTTCCAACTATAGTCATTTCTTTG
CTGCATGATCTGAGTTAGGTCACCAGACTTCTCTGAGCCCCAGTTTCCCC
AGCAGTGTATACGGGCTATGTGGGGAGTATTCAGGAGACAGACAACTCAC
TCGTCAAATCCTCCCCTTCCTGGCCAACAAAGCTGCTGCAACCACAGGGA
TTTCTTCTGTTCAGGTGAGTGTAGGGTGTAGGGAGATTGGTTCAATGTCC
AATTCTTCTGTTTCCCTGGAGATCAGGTTGCCCTTTTTTGGTAGTCTCTC
CAATTCCCTCCTTCCCGGAAGCATGTGACAATCAACAACTTTGTATACTT
AAGTTCAGTGGACCTCAATTTCCTCATCTGTGAAATAAACGGGACTGAAA
AATCATTCTGGCCTCAAGATGCTTTGTTGGGGTGTCTAGGTGCTCCAGGT
GCTTCTGGGAGAGGTGACCTAGTGAGGGATCAGTGGGAATAGAGGTGATA
TTGTGGGGCTTTTCTGGAAATTGCAGAGAGGTGCATCGTTTTTATAATTT
ATGAATTTTTATGTATTAATGTCATCCTCCTGATCTTTTCAGCTGCATTG
GGTAAATCCTTGCCTGCCAGAGTGGGTCAGCGGTGAGCCAGAAAGGGGGC
TCATTCTAACAGTGCTGTGTCCTCCTGGAGAGTGCCAACTCATTCTCCAA
GTAAAAAAAGCCAGATTTGTGGCTCACTTCGTGGGGAAATGTGTCCAGCG
CACCAACGCAGGCGAGGGACTGGGGGAGGAGGGAAGTGCCCTCCTGCAGC
ACGCGAGGTTCCGGGACCGGCTGGCCTGCTGGAACTCGGCCAGGCTCAGC
TGGCTCGGCGCTGGGCAGCCAGGAGCCTGGGCCCCGGGGAGGGCGGTCCC
GGGCGGCGCGGTGGGCCGAGCGCGGGTCCCGCCTCCTTGAGGCGGGCCCG
GGCGGGGCGGTTGTATATCAGGGCCGCGCTGAGCTGCGCCAGCTGAGGTG
TGAGCAGCTGCCGAAGTCAGTTCCTTGTGGAAGCTTGGCATTCCGGTACT
GTTGGTAAAGCCACCATGGCCTCGTACCCCGGCCATCAACACGCGTCTGC
GTTCGACCAGGCTGCGCGTTCTCGCGGCCATAGCAACCGACGTACGGCGT
TGCGCCCTCGCCGGCAGCAAGAAGCCACGGAAGTCCGCCCGGAGCAGAAA
ATGCCCACGCTACTGCGGGTTTATATAGACGGTCCCCACGGGATGGGGAA
AACCACCACCACGCAACTGCTGGTGGCCCTGGGTTCGCGCGACGATATCG
TCTACGTACCCGAGCCGATGACTTACTGGCGGGTGCTGGGGGCTTCCGAG
ACAATCGCGAACATCTACACCACACAACACCGCCTCGACCAGGGTGAGAT
ATCGGCCGGGGACGCGGCGGTGGTAATGACAAGCGCCCAGATAACAATGG
GCATGCCTTATGCCGTGACCGACGCCGTTCTGGCTCCTCATATCGGGGGG
GAGGCTGGGAGCTCACATGCCCCGCCCCCGGCCCTCACCCTCATCTTCGA
CCGCCATCCCATCGCCGCCCTCCTGTGCTACCCGGCCGCGCGGTACCTTA
TGGGCAGCATGACCCCCCAGGCCGTGCTGGCGTTCGTGGCCCTCATCCCG
CCGACCTTGCCCGGCACCAACATCGTGCTTGGGGCCCTTCCGGAGGACAG
ACACATCGACCGCCTGGCCAAACGCCAGCGCCCCGGCGAGCGGCTGGACC
TGGCTATGCTGGCTGCGATTCGCCGCGTTTACGGGCTACTTGCCAATACG
GTGCGGTATCTGCAGTGCGGCGGGTCGTGGCGGGAGGACTGGGGACAGCT
TTCGGGGACGGCCGTGCCGCCCCAGGGTGCCGAGCCCCAGAGCAACGCGG
GCCCACGACCCCATATCGGGGACACGTTATTTACCCTGTTTCGGGCCCCC
GAGTTGCTGGCCCCCAACGGCGACCTGTATAACGTGTTTGCCTGGGCCTT
GGACGTCTTGGCCAAACGCCTCCGTTCCATGCACGTCTTTATCCTGGATT
ACGACCAATCGCCCGCCGGCTGCCGGGACGCCCTGCTGCAACTTACCTCC
GGGATGGTCCAGACCCACGTCACCACCCCCGGCTCCATACCGACGATATG
CGACCTGGCGCGCACGTTTGCCCGGGAGATGGGGGAGGCTAACTGAGAAT
TCGCTAGAGTCGGGGCGGCCGGCCGCTTCGAGCAGACATGATAAGATACA
TTGATGAGTTTGGACAAACCACAACTAGAATGCAGTGAAAAAAATGCTTT
ATTTGTGAAATTTGTGATGCTATTGCTTTATTTGTAACCATTATAAGCTG
CAATAAACAAGTTAACAACAACAATTGCATTCATTTTATGTTTCAGGTTC
AGGGGGAGGTGTGGGAGGTTTTTTAAAGCAAGTAAAACCTCTACAAATGT
GGTAAAATCGATAAGGATCGGCCGCAGGAACCCCTAGTGATGGAGTTGGC
CACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCG
CGCAGCTGCCTGCAGGGGCGCCTGATGCGGTATTTTCTCCTTACGCATCT
GTGCGGTATTTCACACCGCATACGTCAAAGCAACCATAGTACGCGCCCTG
TAGCGGCGCATTAAGCGCGGCGGGTGTGGTGGTTACGCGCAGCGTGACCG
CTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAAC
TTGATTTGGGTGATGGTTCACGTAGTGGGCCATCGCCCTGATAGACGGTT
TTTCGCCCTTTGACGTTGGAGTCCACGTTCTTTAATAGTGGACTCTTGTT
CCAAACTGGAACAACACTCAACCCTATCTCGGGCTATTCTTTTGATTTAT
AAGGGATTTTGCCGATTTCGGCCTATTGGTTAAAAAATGAGCTGATTTAA
CAAAAATTTAACGCGAATTTTAACAAAATATTAACGTTTACAATTTTATG
GTGCACTCTCAGTACAATCTGCTCTGATGCCGCATAGTTAAGCCAGCCCC
GACACCCGCCAACACCCGCTGACGCGCCCTGACGGGCTTGTCTGCTCCCG
GCATCCGCTTACAGACAAGCTGTGACCGTCTCCGGGAGCTGCATGTGTCA
GAGGTTTTCACCGTCATCACCGAAACGCGCGAGACGAAAGGGCCTCGTGA
TACGCCTATTTTTATAGGTTAATGTCATGATAATAATGGTTTCTTAGACG
TCAGGTGGCACTTTTCGGGGAAATGTGCGCGGAACCCCTATTTGTTTATT
TTTCTAAATACATTCAAATATGTATCCGCTCATGAGACAATAACCCTGAT
AAATGCTTCAATAATATTGAAAAAGGAAGAGTATGAGTATTCAACATTTC
CGTGTCGCCCTTATTCCCTTTTTTGCGGCATTTTGCCTTCCTGTTTTTGC
TCACCCAGAAACGCTGGTGAAAGTAAAAGATGCTGAAGATCAGTTGGGTG
CACGAGTGGGTTACATCGAACTGGATCTCAACAGCGGTAAGATCCTTGAG
AGTTTTCGCCCCGAAGAACGTTTTCCAATGATGAGCACTTTTAAAGTTCT
GCTATGTGGCGCGGTATTATCCCGTATTGACGCCGGGCAAGAGCAACTCG
GTCGCCGCATACACTATTCTCAGAATGACTTGGTTGAGTACTCACCAGTC
ACAGAAAAGCATCTTACGGATGGCATGACAGTAAGAGAATTATGCAGTGC
TGCCATAACCATGAGTGATAACACTGCGGCCAACTTACTTCTGACAACGA
TCGGAGGACCGAAGGAGCTAACCGCTTTTTTGCACAACATGGGGGATCAT
GTAACTCGCCTTGATCGTTGGGAACCGGAGCTGAATGAAGCCATACCAAA
CGACGAGCGTGACACCACGATGCCTGTAGCAATGGCAACAACGTTGCGCA
AACTATTAACTGGCGAACTACTTACTCTAGCTTCCCGGCAACAATTAATA
GACTGGATGGAGGCGGATAAAGTTGCAGGACCACTTCTGCGCTCGGCCCT
TCCGGCTGGCTGGTTTATTGCTGATAAATCTGGAGCCGGTGAGCGTGCGT
CTCGCGGTATCATTGCAGCACTGGGGCCAGATGGTAAGCCCTCCCGTATC
GTAGTTATCTACACGACGGGGAGTCAGGCAACTATGGATGAACGAAATAG
ACAGATCGCTGAGATAGGTGCCTCACTGATTAAGCATTGGTAACTGTCAG
ACCAAGTTTACTCATATATACTTTAGATTGATTTAAAACTTCATTTTTAA
TTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGA
TCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGA
GCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAAC
TCTGTAGCACCGCCTACATACCTCGCTCTGCTAATCCTGTTACCAGTGGC
TGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACA
CAGCCCAGCTTGGAGCGAACGACCTACACCGAACTGAGATACCTACAGCG
TGAGCTATGAGAAAGCGCCACGCTTCCCGAAGGGAGAAAGGCGGACAGGT
ATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCT
TTTGCTCACATGT

In the base sequence, Left-ITR is the region represented by the following sequence:

(SEQ ID NO:31)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCACAGAGGGAGTGGCCAACTCCATCACTAGGGGTTCCT,

the 5′ flanking region of the p21 gene is the region represented by the following sequence:

(SEQ ID NO:32)

AAGCTTCCCAGGAACATGCTTGGGCAGCAGGCTGTGGCTCTGATTGGCTT
TCTGGCCGTCAGGAACATGTCCCAACATGTTGAGCTCTGGCATAGAAGAG
GCTGGTGGCTATTTTGTCCTTGGGCTGCCTGTTTTCAGGTGAGGAAGGGG
ATGGTAGGAGACAGGAGACCTCTAAAGACCCCAGGTAAACCTTAGCCTGT
TACTCTGAACAGGGTATGTGATCTGCCAGCAGATCCTTGCGACAGGGCTG
GGATCTGATGCATGTGTGCTTGTGTGAGTGTGTGCTGGGAGTCAGATTCT
GTGTGTGACTTTTAACAGCCTGCTCCCTTGCCTTTTTCAGGGCAGAAGTC
CTCCCTTAGAGTGTGTCTGGGTACACATTCAAGTGCATGGTTGCAAACTT
TTTTTTTTAAAGCACTGAATAGTACTAGACACTTAGTAGGTACTTAAGAA
ATATTGAATGTCGTGGTGGTGGTGAGCTAGAAGTTATAAAAAAAATTCTT
TCCCAAAAACAACAACAAAAAGAATTATTTCATTGTGAAGCTCAGTACCA
CAAAAATTTAAATAATTCATTACAAGCCTTTATTAAAAAAAATTTTCTCC
CCAAAGTAAACAGACAGACAATGTCTAGTCTATTTGAAATGCCTGAAAGC
AGAGGGGCTTCAAGGCAGTGGGAGAAGGTGCCTGTCCTCTGCTGGACATT
TGACAACCAGCCCTTTGGATGGTTTGGATGTATAGGAGCGAAGGTGCAGA
CAGCAGTGGGGCTTAGAGTGGGGTCCTGAGGCTGTGCCGTGGCCTTTCTG
GGGTTTAGCCACAATCCTGGCCTGACTCCAGGGCGAGGCAGGCCAAGGGG
GTCTGCTACTGTGTCCTCCCACCCCTACCTGGGCTCCCATCCCCACAGCA
GAGGAGAAAGAAGCCTGTCCTCCCCGAGGTCAGCTGCGTTAGAGGAAGAA
GACTGGGCATGTCTGGGCAGAGATTTCCAGACTCTGAGCAGCCTGAGATG
TCAGTAATTGTAGCTGCTCCAAGCCTGGGTTCTGTTTTTTAGTGGGATTT
CTGTTCAGATGAACAATCCATCCTCTGCAATTTTTTAAAAGCAAAACTGC
AAATGTTTCAGGCACAGAAAGGAGGCAAAGGTGAAGTCCAGGGGAGGTCA
GGGGTGTGAGGTAGATGGGAGCGGATAGACACATCACTCATTTCTGTGTC
TGTCAGAAGAACCAGTAGACACTTCCAGAATTGTCCTTTATTTATGTCAT
CTCCATAAACCATCTGCAAATGAGGGTTATTTGGCATTTTTGTCATTTTG
GAGCCACAGAAATAAAGGATGACAAGCAGAGAGCCCCGGGCAGGAGGCAA
AAGTCCTGTGTTCCAACTATAGTCATTTCTTTGCTGCATGATCTGAGTTA
GGTCACCAGACTTCTCTGAGCCCCAGTTTCCCCAGCAGTGTATACGGGCT
ATGTGGGGAGTATTCAGGAGACAGACAACTCACTCGTCAAATCCTCCCCT
TCCTGGCCAACAAAGCTCCTGCAACCACAGGGATTTCTTCTGTTCAGGTG
AGTGTAGGGTGTAGGGAGATTGGTTCAATGTCCAATTCTTCTGTTTCCCT
GGAGATCAGGTTGCCCTTTTTTGGTAGTCTCTCCAATTCCCTCCTTCCCG
GAAGCATGTGACAATCAACAACTTTGTATACTTAAGTTCAGTGGACCTCA
ATTTCCTCATCTGTGAAATAAACGGGACTGAAAAATCATTCTGGCCTCAA
GATGCTTTGTTGGGGTGTCTAGGTGCTCCAGGTGCTTCTGGGAGAGGTGA
CCTAGTGAGGGATCAGTGGGAATAGAGGTGATATTGTGGGGCTTTTCTGG
AAATTGCAGAGAGGTGCATCGTTTTTATAATTTATGAATTTTTATGTATT
AATGTCATCCTCCTGATCTTTTCAGCTGCATTGGGTAAATCCTTGCCTGC
CAGAGTGGGTCAGCGGTGAGCCAGAAAGGGGGCTCATTCTAACAGTGCTG
TGTCCTCCTGGAGAGTGCCAACTCATTCTCCAAGTAAAAAAAGCCAGATT
TGTGGCTCACTTCGTGGGGAAATGTGTCCAGCGCACCAACGCAGGCGAGG
GACTGGGGGAGGAGGGAAGTGCCCTCCTGCAGCACGCGAGGTTCCGGGAC
CGGCTGGCCTGCTGGAACTCGGCCAGGCTCAGCTGGCTCGGCGCTGGGCA
GCCAGGAGCCTGGGCCCCGGGGAGGGCGGTCCCGGGCGGCGCGGTGGGCC
GAGCGCGGGTCCCGCCTCCTTGAGGCGGGCCCGGGCGGGGCGGTTGTATA
TCAGGGCCGCGCTGAGCTGCGCCAGCTGAGGTGTGAGCAGCTGCCGAAGT
CAGTTCCTTGTGGAAGCTT,

HSV-tk coding sequence is the region represented by the following sequence:

(SEQ ID NO:33)

ATGGCCTCGTACCCCGGCCATCAACACGCGTCTGCGTTCGACCAGGCTGC
GCGTTCTCGCGGCCATAGCAACCGACGTACGGCGTTGCGCCCTCGCCGGC
AGCAAGAAGCCACGGAAGTCCGCCCGGAGCAGAAAATGCCCACGCTACTG
CGGGTTTATATAGACGGTCCCCACGGGATGGGGAAAACCACCACCACGCA
ACTGCTGGTGGCCCTGGGTTCGCGCGACGATATCGTCTACGTACCCGAGC
CGATGACTTACTGGCGGGTGCTGGGGGCTTCCGAGACAATCGCGAACATC
TACACCACACAACACCGCCTCGACCAGGGTGAGATATCGGCCGGGGACGC
GGCGGTGGTAATGACAAGCGCCCAGATAACAATGGGCATGCCTTATGCCG
TGACCGACGCCGTTCTGGCTCCTCATATCGGGGGGGAGGCTGGGAGCTCA
CATGCCCCGCCCCCGGCCCTCACCCTCATCTTCGACCGCCATCCCATCGC
CGCCCTCCTGTGCTACCCGGCCGCGCGGTACCTTATGGGCAGCATGACCC
CCCAGGCCGTGCTGGCGTTCGTGGCCCTCATCCCGCCGACCTTGCCCGGC
ACCAACATCGTGCTTGGGGCCCTTCCGGAGGACAGACACATCGACCGCCT
GGCCAAACGCCAGCGCCCCGGCGAGCGGCTGGACCTGGCTATGCTGGCTG
CGATTCGCCGCGTTTACGGGCTACTTGCCAATACGGTGCGGTATCTGCAG
TGCGGCGGGTCGTGGCGGGAGGACTGGGGACAGCTTTCGGGGACGGCCGT
GCCGCCCCAGGGTGCCGAGCCCCAGAGCAACGCGGGCCCACGACCCCATA
TCGGGGACACGTTATTTACCCTGTTTCGGGCCCCCGAGTTGCTGGCCCCC
AACGGCGACCTGTATAACGTGTTTGCCTGGGCCTTGGACGTCTTGGCCAA
ACGCCTCCGTTCCATGCACGTCTTTATCCTGGATTACGACCAATCGCCCG
CCGGCTGCCGGGACGCCCTGCTGCAACTTACCTCCGGGATGGTCCAGACC
CACGTCACCACCCCCCGGTCCATACCGACGATATGCGACCTGGCGCGCAC
GTTTGCCCGGGAGATGGGGGAGGCTAACTGA,

the SV40-derived polyadenylation signal is the region represented by the following sequence:

(SEQ ID NO:34)

CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGGTGTGGGAGGTTTTTTAAAGCAAG
TAAAACCTCTACAAATGTGGTA,
and

Right-ITR is the region represented by the following sequence:

(SEQ ID NO:35)

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG.

The pAAV-RC (STRATAGENE AAV Helper-Free System Cat#240071) coding for adeno-associated virus-derived rep and cap genes same as that used in Example 1, was used as a helper plasmid comprising the genes that are necessary for virus reproduction and viral particle formation (rep and cap).

The pHelper (STRATAGENE AAV Helper-Free System Cat#240071) coding for the adenovirus-derived VA, E2A and E4 genes same as that used in Example 1, was used as an adenovirus gene expression plasmid comprising the adenoviral genes that are necessary for the production of adeno-associated virus vector (E2A, E4 and VA).

The constructed or acquired three species of plasmids, pAAV-PtkS, pAAV-RC and pHelper, were co-transfected by the calcium phosphate method using ProFection Mammalian Transfection System (Promega) into 7×10⁶ 293 cells (derived from HEK293 human embryonic kidney cells stably expressing the adenovirus E1 gene) (STRATAGENE AAV Helper-Free System Cat#240071).

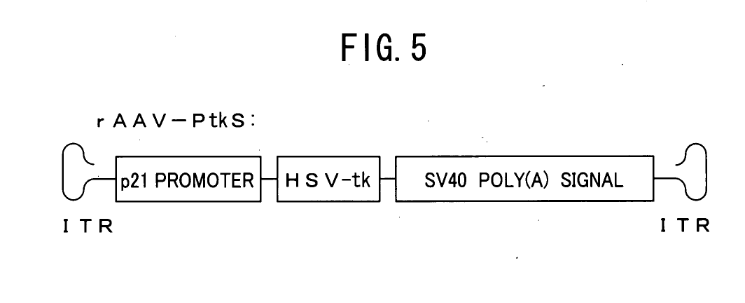

After incubation for three days (culture in 10 ml DMEM containing 10% fetal bovine serum, in an atmosphere at 37° C. containing 5% carbon dioxide), low-dose radiation-inducible viral vector rAAV-PtkS produced in 293 cells was recovered by four freeze-thaw cycles (freezing for 10 minutes in ethanol cooled with dry ice, then, melting in a water bath 37° C.), then centrifuged at 10,000 g for 10 minutes and concentrated. The genome structure of the obtained rAAV-PtkS is shown in FIG. 5. The base sequence of rAAV-PtkS is shown below.

(SEQ ID NO:36)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCTCG
AGATCTGCGATCTAAGTAAGCTTCCCAGGAACATGCTTGGGCAGCAGGCT
GTGGCTCTGATTGGCTTTCTGGCCGTCAGGAACATGTCCCAACATGTTGA
CCTCTGGCATAGAAGAGGCTGGTGGCTATTTTGTCCTTGGGCTGCCTGTT
TTCAGGTGAGGAAGGGGATGGTAGGAGACAGGAGACCTCTAAAGACCCCA
GGTAAACCTTAGCCTGTTACTCTGAACAGGGTATGTGATCTGCCAGCAGA
TCCTTGCGACAGGGCTGGGATCTGATGCATGTGTGCTTGTGTGAGTGTGT
GCTGGGAGTCAGATTCTGTGTGTGACTTTTAACAGCCTGCTCCCTTGCCT
TTTTCAGGGCAGAAGTCCTCCCTTAGAGTGTGTCTGGGTACACATTCAAG
TGCATGGTTGCAAACTTTTTTTTTTAAAGCACTGAATAGTACTAGACACT
TAGTAGGTACTTAAGAAATATTGAATGTCGTGGTGGTGGTGAGCTAGAAG
TTATAAAAAAAATTCTTTCCCAAAAACAACAACAAAAAGAATTATTTCAT
TGTGAAGCTCAGTACCACAAAAATTTAAATAATTCATTACAAGCCTTTAT
TAAAAAAAATTTTCTCCCCAAAGTAAACAGACAGACAATGTCTAGTCTAT
TTGAAATGCCTGAAAGCAGAGGGGCTTCAAGGCAGTGGGAGAAGGTGCCT
GTCCTCTGCTGGACATTTGACAACCAGCCCTTTGGATGGTTTGGATGTAT
AGGAGCGAAGGTGCAGACAGCAGTGGGGCTTAGAGTGGGGTCCTGAGGCT
GTGCCGTGGCCTTTCTGGGGTTTAGCCACAATCCTGGCCTGACTCCAGGG
CGAGGCAGGCCAAGGGGGTCTGCTACTGTGTCCTCCCACCCCTACCTGGG
CTCCCATCCCCACAGCAGAGGAGAAAGAAGCCTGTCCTCCCCGAGGTCAG
CTGCGTTAGAGGAAGAAGACTGGGCATGTCTGGGCAGAGATTTCCAGACT
CTGAGCAGCCTGAGATGTCAGTAATTGTAGCTGCTCCAAGCCTGGGTTCT
GTTTTTTAGTGGGATTTCTGTTCAGATGAACAATCCATCCTCTGCAATTT
TTTAAAAGCAAAACTGCAAATGTTTCAGGCACAGAAAGGAGGCAAAGGTG
AAGTCCAGGGGAGGTCAGGGGTGTGAGGTAGATGGGAGCGGATAGACACA
TCACTCATTTCTGTGTCTGTCAGAAGAACCAGTAGACACTTCCAGAATTG
TCCTTTATTTATGTCATCTCCATAAACCATCTGCAAATGAGGGTTATTTG
GCATTTTTGTCATTTTGGAGCCACAGAAATAAAGGATGACAAGCAGAGAG
CCCCGGGCAGGAGGCAAAAGTCCTGTGTTCCAACTATAGTCATTTCTTTG
CTGCATGATCTGAGTTAGGTCACCAGACTTCTCTGAGCCCCAGTTTCCCC
AGCAGTGTATACGGGCTATGTGGGGAGTATTCAGGACACAGACAACTCAC
TCGTCAAATCCTCCCCTTCCTGGCCAACAAAGCTGCTGCAACCACAGGGA
TTTCTTCTGTTCAGGTGAGTGTAGGGTGTAGGGAGATTGGTTCAATGTCC
AATTCTTCTGTTTCCCTGGAGATCAGGTTGCCCTTTTTTGGTAGTCTCTC
CAATTCCCTCCTTCCCGGAAGCATGTGACAATCAACAACTTTGTATACTT
AAGTTCAGTGGACCTCAATTTCCTCATCTGTGAAATAAACGGGACTGAAA
AATCATTCTGGCCTCAAGATGCTTTGTTGGGGTGTCTAGGTGCTCCAGGT
GCTTCTGGGAGAGGTGACCTAGTGAGGGATCAGTGGGAATAGAGGTGATA
TTGTGGGGCTTTTCTGGAAATTGCAGAGAGGTGCATCGTTTTTATAATTT
ATGAATTTTTATGTATTAATGTCATCCTCCTGATCTTTTCAGCTGCATTG
GGTAAATCCTTGCCTGCCAGAGTGGGTCAGCGGTGAGCCAGAAAGGGGGC
TCATTCTAACAGTGCTGTGTCCTCCTGGAGAGTGCCAACTCATTCTCCAA
GTAAAAAAAGCCAGATTTGTGGCTCACTTCGTGGGGAAATGTGTCCAGCG
CACCAACGCAGGCGAGGGACTGGGGGAGGAGGGAAGTGCCCTCCTGCAGC
ACGCGAGGTTCCGGGACCGGCTGGCCTGCTGGAACTCGGCCAGGCTCAGC
TGGCTCGGCGCTGGGCAGCCAGGAGCCTGGGCCCCGGGGAGGGCGGTCCC
GGGCGGCGCGGTGGGCCGAGCGCGGGTCCCGCCTCCTTGAGGCGGGCCCG
GGCGGGGCGGTTGTATATCAGGGCCGCGCTGAGCTGCGCCAGCTGAGGTG
TGAGCAGCTGCCGAAGTCAGTTCCTTGTGGAAGCTTGGCATTCCGGTACT
GTTGGTAAAGCCACCATGGCCTCGTACCCCGGCCATCAACACGCGTCTGC
GTTCGACCAGGCTGCGCGTTCTCGCGGCCATAGCAACCGACGTACGGCGT
TGCGCCCTCGCCGGCAGCAAGAAGCCACGGAAGTCCGCCCGGAGCAGAAA
ATGCCCACGCTACTGCGGGTTTATATAGACGGTCCCCACGGGATGGGGAA
AACCACCACCACGCAACTGCTGGTGGCCCTGGGTTCGCGCGACGATATCG
TCTACGTACCCGAGCCGATGACTTACTGGCGGGTGCTGGGGGCTTCCGAG
ACAATCGCGAACATCTACACCACACAACACCGCCTCGACCAGGGTGAGAT
ATCGGCCGGGGACGCGGCGGTGGTAATGACAAGCGCCCAGATAACAATGG
GCATGCCTTATGCCGTGACCGACGCCGTTCTGGCTCCTCATATCGGGGGG
GAGGCTGGGAGCTCACATGCCCCGCCCCCGGCCCTCACCCTCATCTTCGA
CCGCCATCCCATCGCCGCCCTCCTGTGCTACCCGGCCGCGCGGTACCTTA
TGGGCAGCATGACCCCCCAGGCCGTGCTGGCGTTCGTGGCCCTCATCCCG
CCGACCTTGCCCGGCACCAACATCGTGCTTGGGGCCCTTCCGGAGGACAG
ACACATCGACCGCCTGGCCAAACGCCAGCGCCCCGGCGAGCGGCTGGACC
TGGCTATGCTGGCTGCGATTCGCCGCGTTTACGGGCTACTTGCCAATACG
GTGCGGTATCTGCAGTGCGGCGGGTCGTGGCGGGAGGACTGGGGACAGCT
TTCGGGGACGGCCGTGCCGCCCCAGGGTGCCGAGCCCCAGAGCAACGCGG
GCCCACGACCCCATATCGGGGACACGTTATTTACCCTGTTTCGGGCCCCC
GAGTTGCTGGCCCCCAACGGCGACCTGTATAACGTGTTTGCCTGGGCCTT
GGACGTCTTGGCCAAACGCCTCCGTTCCATGCACGTCTTTATCCTGGATT
ACGACCAATCGCCCGCCGGCTGCCGGGACGCCCTGCTGCAACTTACCTCC
GGGATGGTCCAGACCCACGTCACCACCCCCGGCTCCATACCGACGATATG
CGACCTGGCGCGCACGTTTGCCCGGGAGATGGGGGAGGCTAACTGAGAAT
TCGCTAGAGTCGGGGCGGCCGGCCGCTTCGAGCAGACATGATAAGATACA
TTGATGAGTTTGGACAAACCACAACTAGAATGCAGTGAAAAAAATGCTTT
ATTTGTGAAATTTGTGATGCTATTGCTTTATTTGTAACCATTATAAGCTG
CAATAAACAAGTTAACAACAACAATTGCATTCATTTTATGTTTCAGGTTC
AGGGGGAGGTGTGGGAGGTTTTTTAAAGCAAGTAAAACCTCTACAAATGT
GGTAAAATCGATAAGGATCGGCCGCAGGAACCCCTAGTGATGGAGTTGGC
CACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCG
CGCAGCTGCCTGCAGG

In the base sequence, Left-ITR is the region represented by the following sequence:

(SEQ ID NO:37)

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT,

the 5′ flanking region of the p21 gene is the region represented by the following sequence:

(SEQ ID NO:38)

AAGCTTCCCAGGAACATGCTTGGGCAGCAGGCTGTGGCTCTGATTGGCTT
TCTGGCCGTCAGGAACATGTCCCAACATGTTGAGCTCTGGCATAGAAGAG
GCTGGTGGCTATTTTGTCCTTGGGCTGCCTGTTTTCAGGTGAGGAAGGGG
ATGGTAGGAGACAGGAGACCTCTAAAGACCCCAGGTAAACCTTAGCCTGT
TACTCTGAACAGGGTATGTGATCTGCCAGCAGATCCTTGCGACAGGGCTG
GGATCTGATGCATGTGTGCTTGTGTGAGTGTGTGCTGGGAGTCAGATTCT
GTGTGTGACTTTTAACAGCCTGCTCCCTTGCCTTTTTCAGGGCAGAAGTC
CTCCCTTAGAGTGTGTCTGGGTACACATTCAAGTGCATGGTTGCAAACTT
TTTTTTTTAAAGCACTGAATAGTACTAGACACTTAGTAGGTACTTAAGAA
ATATTGAATGTCGTGGTGGTGGTGAGCTAGAAGTTATAAAAAAAATTCTT
TCCCAAAAACAACAACAAAAAGAATTATTTCATTGTGAAGCTCAGTACCA
CAAAAATTTAAATAATTCATTACAAGCCTTTATTAAAAAAAATTTTCTCC
CCAAAGTAAACAGACAGACAATGTCTAGTCTATTTGAAATGCCTGAAAGC
AGAGGGGCTTCAAGGCAGTGGGAGAAGGTGCCTGTCCTCTGCTGGACATT
TGACAACCAGCCCTTTGGATGGTTTGGATGTATAGGAGCGAAGGTGCAGA
CAGCAGTGGGGCTTAGAGTGGGGTCCTGAGGCTGTGCCGTGGCCTTTCTG
GGGTTTAGCCACAATCCTGGCCTGACTCCAGGGCGAGGCAGGCCAAGGGG
GTCTGCTACTGTGTCCTCCCACCCCTACCTGGGCTCCCATCCCCACAGCA
GAGGAGAAAGAAGCCTGTCCTCCCCGAGGTCAGCTGCGTTAGAGGAAGAA
GACTGGGCATGTCTGGGCAGAGATTTCCAGACTCTGAGCAGCCTGAGATG
TCAGTAATTGTAGCTGCTCCAAGCCTGGGTTCTGTTTTTTAGTGGGATTT
CTGTTCAGATGAACAATCCATCCTCTGCAATTTTTTAAAAGCAAAACTGC
AAATGTTTCAGGCACAGAAAGGAGGCAAAGGTGAAGTCCAGGGGAGGTCA
GGGGTGTGAGGTAGATGGGAGCGGATAGACACATCACTCATTTCTGTGTC
TGTCAGAAGAACCAGTAGACACTTCCAGAATTGTCCTTTATTTATGTCAT
CTCCATAAACCATCTGCAAATGAGGGTTATTTGGCATTTTTGTCATTTTG
GAGCCACAGAAATAAAGGATGACAAGCAGAGAGCCCCGGGCAGGAGGCAA
AAGTCCTGTGTTCCAACTATAGTCATTTCTTTGCTGCATGATCTGAGTTA
GGTCACCAGACTTCTCTGAGCCCCAGTTTCCCCAGCAGTGTATACGGGCT
ATGTGGGGAGTATTCAGGAGACAGACAACTCACTCGTCAAATCCTCCCCT
TCCTGGCCAACAAAGCTGCTGCAACCACAGGGATTTCTTCTGTTCAGGTG
AGTGTAGGGTGTAGGGAGATTGGTTCAATGTCCAATTCTTCTGTTTCCCT
GGAGATCAGGTTGCCCTTTTTTGGTAGTCTCTCCAATTCCCTCCTTCCCG
GAAGCATGTGACAATCAACAACTTTGTATACTTAAGTTCAGTGGACCTCA
ATTTCCTCATCTGTGAAATAAACGGGACTGAAAAATCATTCTGGCCTCAA
GATGCTTTGTTGGGGTGTCTAGGTGCTCCAGGTGCTTCTGGGAGAGGTGA
CCTAGTGAGGGATCAGTGGGAATAGAGGTGATATTGTGGGGCTTTTCTGG
AAATTGCAGAGAGGTGCATCGTTTTTATAATTTATGAATTTTTATGTATT
AATGTCATCCTCCTGATCTTTTCAGCTGCATTGGGTAAATCCTTGCCTGC
CAGAGTGGGTCAGCGGTGAGCCAGAAAGGGGGCTCATTCTAACAGTGCTG
TGTCCTCCTGGAGAGTGCCAACTCATTCTCCAAGTAAAAAAAGCCAGATT
TGTGGCTCACTTCGTGGGGAAATGTGTCCAGCGCACCAACGCAGGCGAGG
GACTGGGGGAGGAGGGAAGTGCCCTCCTGCAGCACGCGAGGTTCCGGGAC
CGGCTGGCCTGCTGGAACTCGGCCAGGCTCAGCTGGCTCGGCGCTGGGCA
GCCAGGAGCCTGGGCCCCGGGGAGGGCGGTCCCGGGCGGCGCGGTGGGCC
GAGCGCGGGTCCCGCCTCCTTGAGGCGGGCCCGGGCGGGGCGGTTGTATA
TCAGGGCCGCGCTGAGCTGCGCCAGCTGAGGTGTGAGCAGCTGCCGAAGT
CAGTTCCTTGTGGAAGCTT,

the HSV-tk coding sequence is the region represented by the following sequence:

(SEQ ID NO: 39)

ATGGCCTCGTACCCCGGCCATCAACACGCGTCTGCGTTCGACCAGGCTGC
GCGTTCTCGCGGCCATAGCAACCGACGTACGGCGTTGCGCCCTCGCCGGC
AGCAAGAAGCCACGGAAGTCCGCCCGGAGCAGAAAATGCCCACGCTACTG
CGGGTTTATATAGACGGTCCCCACGGGATGGGGAAAACCACCACCACGCA
ACTGCTGGTGGCCCTGGGTTCGCGCGACGATATCGTCTACGTACCCGAGC
CGATGACTTACTGGCGGGTGCTGGGGGCTTCCGAGACAATCGCGAACATC
TACACCACACAACACCGCCTCGACCAGGGTGAGATATCGGCCGGGGACGC
GGCGGTGGTAATGACAAGCGCCCAGATAACAATGGGCATGCCTTATGCCG
TGACCGACGCCGTTCTGGCTCCTCATATCGGGGGGGAGGCTGGGAGCTCA
CATGCCCCGCCCCCGGCCCTCACCCTCATCTTCGACCGCCATCCCATCGC
CGCCCTCCTGTGCTACCCGGCCGCGCGGTACCTTATGGGCAGCATGACCC
CCCAGGCCGTGCTGGCGTTCGTGGCCCTCATCCCGCCGACCTTGCCCGGC
ACCAACATCGTGCTTGGGGCCCTTCCGGAGGACAGACACATCGACCGCCT
GGCCAAACGCCAGCGCCCCGGCGAGCGGCTGGACCTGGCTATGCTGGCTG
CGATTCGCCGCGTTTACGGGCTACTTGCCAATACGGTGCGGTATCTGCAG
TGCGGCGGGTCGTGGCGGGAGGACTGGGGACAGCTTTCGGGGACGGCCGT
GCCGCCCCAGGGTGCCGAGCCCCAGAGCAACGCGGGCCCACGACCCCATA
TCGGGGACACGTTATTTACCCTGTTTCGGGCCCCCGAGTTGCTGGCCCCC
AACGGCGACCTGTATAACGTGTTTGCCTGGGCCTTGGACGTCTTGGCCAA
ACGCCTCCGTTCCATGCACGTCTTTATCCTGGATTACGACCAATCGCCCG
CCGGCTGCCGGGACGCCCTGCTGCAACTTACCTCCGGGATGGTCCAGACC
CACGTCACCACCCCCGGCTCCATACCGACGATATGCGACCTGGCGCGCAC
GTTTGCCCGGGAGATGGGGGAGGCTAACTGA,

the SV40-derived polyadenylation signal is the region represented by the following sequence:

(SEQ ID NO: 40)

CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGGTGTGGGAGGTTTTTTAAAGCAAG
TAAAACCTCTACAAATGTGGTA,
and

Right-ITR is the region represented by the following sequence:

AGGAACCCCTAGTGATGGAGTTGGCCACTCCCTCTCTGCGCGCTCGCTCGCTCACTGAGGCC GGGCGACCAAAGGTCGCCCGACGCCCGGGCTTTGCCCGGGCGGCCTCAGTGAGCGAGCGAGC GCGCAGCTGCCTGCAGG (SEQ ID NO: 41). Therefore, rAAV-PtkS contained a DNA sequence comprising (a) Left-ITR, (b) p53 target gene promoter sequence, (c) therapeutic gene sequence (HSV-tk gene), (d) polyadenylation signal sequence and (e) Right-ITR, from the five prime end side to the three prime end side in the order of (a), (d), (c), (b), (e).

### Example 5

### Pharmaceutical Composition Comprising Integration-Type Low-Dose Radiation-Inducible Viral Vector

The low-dose radiation-inducible viral vector rAAV-PtkS produced in 293 cells was recovered by four freeze-thaw cycles, then centrifuged at 10,000 g for 10 minutes and concentrated. The obtained concentrate contained the low-dose radiation-inducible viral vector rAAV-PtkS and a buffer solution.

### Example 6

### Transduction Using Integration-Type Viral Vector

(1) Transduction

MCF-7 cells, which are human breast cancer cells expressing p53, were used as host cells.

MCF-7 cells were transduced with viral vector (multiplicity of infection: 5.5×10³) by mixing 0.25 ml of pharmaceutical composition prepared in Example 5 (viral inoculum) (containing 5.5×10⁸ rAAV-PtkS viral particles) and 10⁵ MCF-7 cells in a 12-well microplate, and incubating for 24 hours (culturing in 2 ml of RPMI1640 containing 10% fetal bovine serum, in an atmosphere at 37° C. containing 5% carbon dioxide). Then, cells were washed with PBS to remove the viral inoculum and cultured in an RPMI1640 culture medium (Life Technologies) supplemented with 10% FBS (JRH), 100 unit/ml penicillin and 100 μg/ml streptomycin (Life Technologies), at 37° C., under 5% CO₂ in a humidified atmosphere.

(2) Evaluation of Induced Expression of HSV-tk Gene by X-Ray Irradiation with the Quantity of mRNA Expression of the HSV-tk Gene as Indicator

In the present example, induced expression of HSV-tk gene by X-ray irradiation was evaluated by RT-PCR, with the quantity of mRNA expression of the HSV-tk gene as indicator.

MCF-7 cells cultured for approximately 3 months after being transduced according to Example 6 (culture for 70 to 80 days+cryo-conservation after culture+culture for 10 to 20 days after melting) were irradiated with 5 Gy X-ray. The dose of 5 Gy was used in order to distinctly observe the changes in the amount of HSV-tk gene expression due to X-ray irradiation. X-ray was generated from a Pantak unit fitted with a 0.5 mm copper filter and a 0.5 mm aluminum filter, and operating at 200 kVp and 20 mA. In addition, irradiation was carried out at a dose rate of approximately 1 Gy/minute.

Samples that were not X-ray irradiated (0 Gy) served as controls.

After X-ray irradiation, the amounts of mRNA expression of the HSV-tk gene and the actin gene of the transduced MCF-7 cells were measured by RT-PCR. The actin gene is an endogenous gene to MCF-7 cells and was used as a control for the transduced HSV-tk gene. RT-PCR was carried out according to the procedure described below.

After isolating the total RNA from transduced MCF-7 cells using TRIzol (Invitrogen), poly(A)⁺ RNA was isolated using Oligotex-dT30 (Japanese Roche). With the obtained poly(A)⁺ RNA as the template, cDNA was synthesized using a cDNA synthesis kit (Life Sciences, Inc.). Thereafter, With the obtained cDNA as the template, PCR amplification was carried out (reaction condition: one minute at 95° C., 30 seconds at 60° C., one minute at 72° C.) using PCR primers specific to the HSV-tk coding sequence or the actin gene, LA Taq polymerase (Takara Shuzo), and GC buffer (Takara Shuzo).

The sequences of the PCR primers used are as follows:

| | |
|---|---|
| HSV-tk: | |
| CGGAGCAGAAAATGCCCACG | (SEQ ID NO: 42) |
| TGCTGCCCATAAGGTACCGC | (SEQ ID NO: 43) |
| Actin: | |
| GTAGCCATCCAGGCTGTGTT | (SEQ ID NO: 44) |
| CAGTGAGGCCAGGATAGAGC | (SEQ ID NO: 45) |

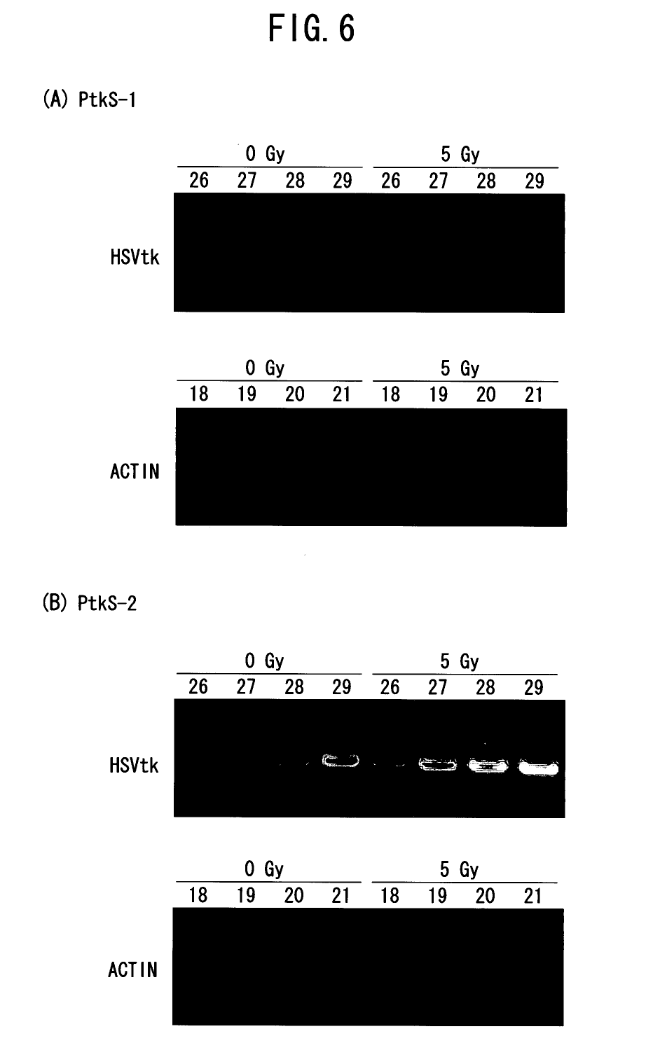

Experiments according to the above procedure were carried out twice. The results are shown in FIG. 6. In FIG. 6, the first test result is indicated as (A) PtkS-1 and the second test result is indicated as (B) PtkS-2.

In FIGS. 6 (A) and (B), the numbers 26 to 29 for the HSV-tk gene and the numbers 18 to 21 for the actin genes indicate the number of the respective RT-PCR cycles.

The number of RT-PCR cycles carried out for the HSV-tk gene (26 to 29) was greater than the number of RT-PCR cycles carried out for the actin gene (18 to 21), since a greater number of cycles was necessary to carry out evaluation of HSV-tk gene expression induced by X-ray irradiation, owing to the extremely low amount of expression of the HSV-tk gene, which is an exogenous gene, compared to the amount of expression of the actin gene (mRNA quantity), which is an endogenous gene.

In FIG. 6 (A), compared to the actin gene, for which the amount of expression is known to not change due to X-ray irradiation, an increase in the amount of expression by X-ray irradiation was observed for the HSV-tk gene.

FIG. 6 (B) showed the same tendency as FIG. 6 (A). Consequently, reproducible results were obtained for the increase in the amount of expression of the HSV-tk gene by X-ray irradiation.

These results show that intracellular expression of the HSV-tk gene was induced by X-ray irradiation in MCF-7 cells into which the HSV-tk gene was transduced with the vector of the present invention rAAV-PtkS.

(3) Evaluation of Induced Expression of HSV-tk Gene by Low-Dose X-Ray Irradiation with the Survival Rate of Transduced Cells as Indicator

The herpes simplex virus thymidine kinase (HSV-tk), which is the expression product of the transduced HSV-tk gene, can activate ganciclovir to exert inhibitory action on DNA synthesis and kill transduced cells. Thus, in the present example, induced expression of HSV-tk gene by low-dose X-ray irradiation was evaluated with, as indicator, the survival rate when transduced cells were irradiated with low-dose X-ray in the presence of ganciclovir.

Two samples of HSV-tk transgenic MCF-7 cells (PtkS-1 and PtkS-2) cultured for approximately 3 months after being transduced according to Example 6 (culture for 70 to 80 days+cryo-conservation after culture+culture for 10 to 20 days after melting), as well as luciferase transgenic MCF-7 cell sample (PLS) cultured for approximately 3 months after being transduced according to Example 3 (culture for 66 days+cryo-conservation after culture+culture for 10 to 20 days after melting) were used as samples.

Each cell sample was irradiated in the presence of 1 mg/ml of ganciclovir (InvivoGen) with low-dose X-ray of 1 Gy, twice daily (however, irradiation was once a day on the ganciclovir administration day), for a total of 5 days (total of 9 Gy). X-ray was generated from a Pantak unit fitted with a 0.5 mm copper filter and a 0.5 mm aluminum filter, and operating at 200 kVp and 20 mA. In addition, irradiation was carried out at a dose rate of 1 Gy/minute. As controls, the same X-ray irradiation was carried out on each cell sample in the absence of ganciclovir.

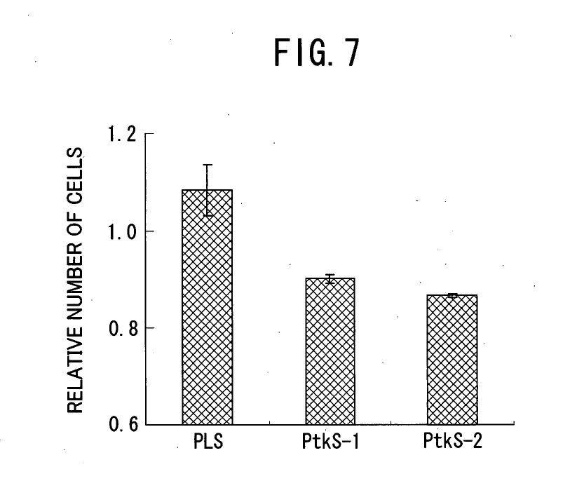

Two days after X-ray irradiation, the number of living cells was counted by the MTT method using the Promega kit “CellTiter96 Non-Radioactive Cell Proliferation Assay”. The result is shown in FIG. 7. The vertical axis in FIG. 7 indicates the ratio of the number of surviving cells in the presence of ganciclovir versus number of surviving cells in the absence of ganciclovir (relative cell number).

The two samples of HSV-tk transgenic cell (PtkS-1 and PtkS-2) demonstrated a significant decrease in the survival rate due to the low-dose X-ray irradiation in the presence of ganciclovir. On the other hand, the sample of luciferase transgenic cell (PLS) did not show a decrease in the survival rate. This result is thought to show that, when MCF-7 cells (human breast cancer cells) transduced with the HSV-tk gene by the vector of the present invention rAAV-PtkS were irradiated with low-dose X-ray in the presence of ganciclovir, the expression of HSV-tk gene was induced inside the cells to produce HSV-tk, and ganciclovir exerted cytotoxicity (DNA synthesis inhibitory action) via this produced HSV-tk, resulting in MCF-7 cell cells being killed.

From the above, it is understood that the integration-type viral vector of the present invention, when introduced into a host, can exert a therapeutic effect by irradiation of low-dose radiation.

### INDUSTRIAL APPLICABILITY

As shown in the examples described above, the integration-type viral vector of the present invention allows therapeutic gene expression in a host cell to be induced to a high degree by irradiation of low-dose radiation. Therefore, the present invention is usable in gene therapy.

1. An integration-type low-dose radiation-inducible viral vector comprising a DNA sequence comprising a p53 target gene promoter sequence and a therapeutic gene sequence.

2. The vector according to claim 1, comprising a DNA sequence comprising
(a) a Left-ITR,
(b) a p53 target gene promoter sequence,
(c) a therapeutic gene sequence,
(d) a polyadenylation signal sequence, and
(e) a Right-ITR,
from the five prime end side to the three prime end side, in the order of (a), (b), (c), (d), (e).

3. The vector according to claim 1, comprising a DNA sequence comprising
(a) a Left-ITR,
(d) a complementary sequence to a polyadenylation signal sequence,
(c) a complementary sequence to a therapeutic gene sequence,
(b) a complementary sequence to a p53 target gene promoter sequence, and
(e) a Right-ITR,
from the five prime end side to the three prime end side, in the order of (a), (d), (c), (b), (e).

4. The vector according to claim 1, which is a vector derived from an adeno-associated virus.

5. The vector according to claim 1, wherein said p53 target gene promoter sequence is the p21 gene promoter sequence.

6. The vector according to claim 1, which has a herpes simplex virus thymidine kinase (HSV-tk) gene sequence as said therapeutic gene sequence.

7. The viral vector according to claim 1, which is a vector for gene therapy use.

8. A pharmaceutical composition for treating a disease treatable by gene therapy, comprising the integration-type low-dose radiation-inducible viral vector according to claim 1.

9. The pharmaceutical composition according to claim 8, wherein said disease treatable by gene therapy is cancer.

10. A gene therapy method for disease treatable by gene therapy, comprising the steps of:
(1) providing a pharmaceutical composition comprising an integration-type low-dose radiation-inducible viral vector comprising a DNA sequence comprising a p53 target gene promoter sequence and a therapeutic gene sequence;
(2) administering said pharmaceutical composition to a patient having disease treatable by gene therapy; and
(3) irradiating a site requiring the expression of the therapeutic gene on said patient, with a dose of radiation sufficient to express the DNA sequence of the vector integrated in the chromosome of said patient.

11. The method according to claim 10, wherein said integration-type low-dose radiation-inducible viral vector comprises a DNA sequence comprising,
(a) a Left-ITR,
(b) a p53 target gene promoter sequence,
(c) a therapeutic gene sequence,
(d) a polyadenylation signal sequence, and
(e) a Right-ITR,
from the five prime end side to the three prime end side, in the order of (a), (b), (c), (d), (e).

12. The method according to claim 10, wherein said integration-type low-dose radiation-inducible viral vector comprises a DNA sequence comprising
(a) a Left-ITR,
(d) a complementary sequence to a polyadenylation signal sequence,
(c) a complementary sequence to a therapeutic gene sequence,
(b) a complementary sequence to a p53 target gene promoter sequence, and
(e) a Right-ITR
from the five prime end side to the three prime end side, in the order of (a), (d), (c), (b), (e).

13. The method according to claim 10, wherein said integration-type low-dose radiation-inducible viral vector has a herpes simplex virus thymidine kinase (HSV-tk) gene sequence as said therapeutic gene sequence.

14. A cancer gene therapy method, comprising the steps of:
(1) providing a pharmaceutical composition comprising an integration-type low-dose radiation-inducible viral vector comprising a DNA sequence comprising a p53 target gene promoter sequence and a therapeutic gene sequence;
(2) administering said pharmaceutical composition to a cancer patient;
(3) irradiating cancer foci of said patient with a dose of radiation sufficient to express the DNA sequence of the vector integrated in the chromosome of said patient; and
(4) irradiating the site expressing said therapeutic gene with a dose of radiation sufficient to treat cancer.

15. The method according to claim 14, wherein said integration-type low-dose radiation-inducible viral vector comprises a DNA sequence comprising
(a) a Left-ITR,
(b) a p53 target gene promoter sequence,
(c) a therapeutic gene sequence,
(d) a polyadenylation signal sequence, and
(e) a Right-ITR,
from the five prime end side to the three prime end side, in the order of (a), (b), (c), (d), (e).

16. The method according to claim 14, wherein said integration-type low-dose radiation-inducible viral vector comprises a DNA sequence comprising
(a) a Left-ITR,
(d) a complementary sequence to a polyadenylation signal sequence,
(c) a complementary sequence to a therapeutic gene sequence,
(b) a complementary sequence to a p53 target gene promoter sequence, and
(e) a Right-ITR
from the five prime end side to the three prime end side, in the order of (a), (d), (c), (b), (e).

17. The method according to claim 14, wherein said integration-type low-dose radiation-inducible viral vector has a herpes simplex virus thymidine kinase (HSV-tk) gene sequence as said therapeutic gene sequence.
